SEQUENCE LISTING (74 sequences)

SEQ ID NO: 1 - protein, 355 aa, Homo sapiens
MDYTLDLSVTTVTDYYYPDIFSSPCDAELIQTNGKLLLAVFYCLLFVFSLLGNSLVILVLVVCKKLRSITDVYLLNLALSDLLFVFSFPFQTYYLLDQWVFGTVMCKVVSGFYYIGFYSSMFFITLMSVDRYLAVVHAVYALKVRTIRMGTTLCLAVWLTAIMATIPLLVFYQVASEDGVLQCYSFYNQQTLKWKIFTNFKMNILGLLIPFTIFMFCYIKILHQLKRCQNHNKTKAIRLVLIVVIASLLFWVPFNVVLFLTSLHSMHILDGCSISQQLTYATHVTEIISFTHCCVNPVIYAFVGEKFKKHLSEIFQKSCSQIFNYLGRQMPRESCEKSSSCQQHSSRSSSVDYIL

SEQ ID NO: 2 - protein, 7 aa, Homo sapiens
YYYPDIF

SEQ ID NO: 3 - skipped

SEQ ID NO: 4 - protein, 121 aa, Homo sapiens
VVQLQQSGPVLVKPGASVKMSCAASGYTFTDNYINWVKQRHGKSLEWIGVINPYNGLTAYDQNFKGKATLTVDKSSSTAYMALNSLTSEASAVYYCARRYGGTPVRYFDVWGTGTTVTVSS

SEQ ID NO: 5 - skipped

SEQ ID NO: 6 - protein, 121 aa, Homo sapiens; identical to SEQ ID NO: 4

SEQ ID NO: 7 - skipped

SEQ ID NO: 8 - protein, 116 aa, Homo sapiens
QVQLVESGGGVVQPGRSLRLSCAVSGLTFSSYAMHWVRQAPGKGLEWVAVISYDGNKKYNADSVKGRFTISRDNSKNTLYLQMNSLRAEDTAVYYCARAEGKGDYWGQGTLVTVSS

SEQ ID NO: 9 - protein, 116 aa, Homo sapiens
QVQLVQSGAEVKKPGASVKVSCKASGYTFTDSEMHWVRQATGQGLEWMGAIQPETGGTAYNQKFKARVTMTRDTSISTAYMELSSLRSEDTAVYYCARRRRNFDYWGQGTLVTVSS

SEQ ID NO: 10 - skipped

SEQ ID NO: 11 - protein, 112 aa, Homo sapiens
DVVLTQTPLTLSVTIGQPASISCKSSQSLLDSSGKTYLNWLLQRPGQSPKRLIYLVSKLDSGVPDRFSGSGSGTDFTLKFSRVEAEDLGVYYCWQATHFPWSFGGGSKLEVK

SEQ ID NO: 12 - skipped

SEQ ID NO: 13 - protein, 107 aa, Homo sapiens
DIQMTQTASSLSASLGARVTISCRASQDISNYLNWYQQKPDGTFELLIYYTSRLHSGVPSRFSGSGSGTDYSLTITNLEQEDIATYFCQQGETLPWTFGGGTKLEIK

SEQ ID NO: 14 - skipped

SEQ ID NO: 15 - protein, 107 aa, Homo sapiens
EIVLTQSPATLSLSPGERATLSCRASQSVSSYLVWYQQKPGQAPRLLIYDASNRATGIPARFSGSGSGTDFTLTISSLEPEDFAVYYCQQRSNWPRTFGQGTKVEIK

SEQ ID NO: 16 - protein, 112 aa, Homo sapiens
DIVMTQTPLSLSVTPGQPASISCRSSQSLFHSSGNTYLHWYLQKPGQPPQLLIYKVSNRFSGVPDRFSGSGSGTDFTLKISRVEAEDVGVYYCSQSTHVPFTFGQGTKLEIK

SEQ ID NO: 17-22 - skipped (6 entries)

SEQ ID NO: 23 - protein, 10 aa, Homo sapiens
GYTFTDNYIN

SEQ ID NO: 24 - protein, 19 aa, Homo sapiens
VINPYNGLTAYDQNFKGKA

SEQ ID NO: 25 - protein, 12 aa, Homo sapiens
RYGGTPVRYFDV

SEQ ID NO: 26 - protein, 16 aa, Homo sapiens
KSSQSLLDSSGKTYLN

SEQ ID NO: 27 - protein, 7 aa, Homo sapiens
LVSKLDS

SEQ ID NO: 28 - protein, 9 aa, Homo sapiens
WQATHFPWS

SEQ ID NO: 29-34 - skipped (6 entries)

SEQ ID NO: 35 - protein, 10 aa, Homo sapiens
GYTFTDNYIN

SEQ ID NO: 36 - protein, 19 aa, Homo sapiens
VINPYNGLTAYDQNFKGKA

SEQ ID NO: 37 - protein, 12 aa, Homo sapiens
RYGGTPVRYFDV

SEQ ID NO: 38 - protein, 11 aa, Homo sapiens
RASQDISNYLN

SEQ ID NO: 39 - protein, 7 aa, Homo sapiens
YTSRLHS

SEQ ID NO: 40 - protein, 9 aa, Homo sapiens
QQGETLPWT

SEQ ID NO: 41-46 - skipped (6 entries)

SEQ ID NO: 47 - protein, 10 aa, Homo sapiens
GLTFSSYAMH

SEQ ID NO: 48 - protein, 19 aa, Homo sapiens
VISYDGNKKYNADSVKGRF

SEQ ID NO: 49 - protein, 7 aa, Homo sapiens
AEGKGDY

SEQ ID NO: 50 - protein, 11 aa, Homo sapiens
RASQSVSSYLV

SEQ ID NO: 51 - protein, 7 aa, Homo sapiens
DASNRAT

SEQ ID NO: 52 - protein, 9 aa, Homo sapiens
QQRSNWPRT

SEQ ID NO: 53 - protein, 10 aa, Homo sapiens
GYTFTDSEMH

SEQ ID NO: 54 - protein, 17 aa, Homo sapiens
AIQPETGGTAYNQKFKA

SEQ ID NO: 55 - protein, 7 aa, Homo sapiens
RRRNFDY

SEQ ID NO: 56 - protein, 16 aa, Homo sapiens
RSSQSLFHSSGNTYLH

SEQ ID NO: 57 - protein, 7 aa, Homo sapiens
KVSNRFS

SEQ ID NO: 58 - protein, 9 aa, Homo sapiens
SQSTHVPFT

SEQ ID NO: 59 - skipped

SEQ ID NO: 60 - protein, 444 aa, Homo sapiens
VVQLQQSGPVLVKPGASVKMSCAASGYTFTDNYINWVKQRHGKSLEWIGVINPYNGLTAYDQNFKGKATLTVDKSSSTAYMALNSLTSEASAVYYCARRYGGTPVRYFDVWGTGTTVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPG

SEQ ID NO: 61 - skipped

SEQ ID NO: 62 - protein, 444 aa, Homo sapiens; identical to SEQ ID NO: 60

SEQ ID NO: 63 - skipped

SEQ ID NO: 64 - protein, 445 aa, Homo sapiens
QVQLVESGGGVVQPGRSLRLSCAVSGLTFSSYAMHWVRQAPGKGLEWVAVISYDGNKKYNADSVKGRFTISRDNSKNTLYLQMNSLRAEDTAVYYCARAEGKGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 65 - protein, 445 aa, Homo sapiens
QVQLVQSGAEVKKPGASVKVSCKASGYTFTDSEMHWVRQATGQGLEWMGAIQPETGGTAYNQKFKARVTMTRDTSISTAYMELSSLRSEDTAVYYCARRRRNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 66 - skipped

SEQ ID NO: 67 - protein, 219 aa, Homo sapiens
DVVLTQTPLTLSVTIGQPASISCKSSQSLLDSSGKTYLNWLLQRPGQSPKRLIYLVSKLDSGVPDRFSGSGSGTDFTLKFSRVEAEDLGVYYCWQATHFPWSFGGGSKLEVKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 68 - skipped

SEQ ID NO: 69 - protein, 214 aa, Homo sapiens
DIQMTQTASSLSASLGARVTISCRASQDISNYLNWYQQKPDGTFELLIYYTSRLHSGVPSRFSGSGSGTDYSLTITNLEQEDIATYFCQQGETLPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 70 - skipped

SEQ ID NO: 71 - protein, 214 aa, Homo sapiens
EIVLTQSPATLSLSPGERATLSCRASQSVSSYLVWYQQKPGQAPRLLIYDASNRATGIPARFSGSGSGTDFTLTISSLEPEDFAVYYCQQRSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 72 - protein, 219 aa, Homo sapiens
DIVMTQTPLSLSVTPGQPASISCRSSQSLFHSSGNTYLHWYLQKPGQPPQLLIYKVSNRFSGVPDRFSGSGSGTDFTLKISRVEAEDVGVYYCSQSTHVPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 11 aa, Homo sapiens
VTDYYYPDIFS

SEQ ID NO: 74 - protein, 35 aa, Homo sapiens
MDYTLDLSVTTVTDYYYPDIFSSPCDAELIQTNGK

(19) United States
(12) Patent Application Publication
(10) Pub. No.: US 2026/0028409 A1
(43) Pub. Date: Jan. 29, 2026

(54) ANTIBODIES THAT BIND TO HUMAN CCR8

(71) Applicant: Bristol-Myers Squibb Company, Princeton, NJ (US)

(72) Inventors: Sharmila Sambanthamoorthy, Belmont, CA (US); Joseph Richard Campbell, Redwood City, CA (US); Anan Chuntharapai, Daly City, CA (US); Felix Findeisen, San Francisco, CA (US); Renu Jain, Redwood City, CA (US); Ruth Yin-Zong Lan, Fremont, CA (US); John K. Lee, Alameda, CA (US); Linda Liang, Mountain View, CA (US); Kai Lu, Foster City, CA (US); Paul Blaine Mesko, Redwood City, CA (US); Isha Pradhan, Newtown, PA (US); Pavel Strop, San Mateo, CA (US); Sayumi Yamazoe, Mountain View, CA (US)

(73) Assignee: Bristol-Myers Squibb Company, PRINCETON, NJ (US)

(21) Appl. No.: 18/868,665

(22) PCT Filed: May 23, 2023
(86) PCT No.: PCT/US2023/067354

§ 371 Date: Nov. 22, 2024

Related U.S. Application Data
(60) Provisional application No. 63/365,255, filed on May 24, 2022.

(51) Int. Cl. C07K 16/28; A61K 39/00; A61P 35/00; C07K 14/725; G01N 33/50

(52) U.S. Cl. CPC C07K 16/2866; A61P 35/00; C07K 14/7051; G01N 33/5011; A61K 2039/505; C07K 2317/24; C07K 2317/34; C07K 2317/92; G01N 2333/7158

(57) ABSTRACT

This disclosure provides isolated antibodies that specifically bind to the C-C Motif Chemokine Receptor 8 (CCR8) expressed on the surface of a cell and exhibit various functional properties, including properties that are desirable in a diagnostic antibody. These properties include binding with high affinity and specificity to CCR8-expressing cells, such as tumor-infiltrating, activated CD4⁺FOXP3^{high} Tregs, and binding to a human CCR8 (hCCR8) epitope outside the N-terminal domain of hCCR8 to which most therapeutic anti-CCR8 antibodies bind.

Throughout this application, various publications are referenced in parentheses by author name and date, or by patent No. or Patent Publication No. Full citations for these publications may be found near the end of the specification immediately preceding the claims. The disclosures of these publications are hereby incorporated in their entireties by reference into this application in order to more fully describe the state of the art as known to those skilled therein as of the date of the invention described and claimed herein. However, these disclosures are incorporated into the present application only to the extent that no conflict exists between the information incorporated by reference and the information provided by explicit disclosure in the present application. Notably, the citation of a reference herein should not be construed as an acknowledgement that such reference is prior art to the present invention.

### CROSS REFERENCE TO RELATED APPLICATIONS

This application claims the benefit of U.S. Provisional Application No. 63/365,255, filed May 24, 2022, the entire content of which is hereby incorporated herein by reference.

### SEQUENCE LISTING

The present application contains a Sequence Listing which has been submitted electronically via Patent Center in ST.26-compliant XML format and is hereby incorporated herein by reference in its entirety. The ST.26 copy was created on May 18, 2023, is named 20230518_SEQL_13429WOPCT.xml, and is 48,452 bytes in size.

### FIELD OF THE INVENTION

The present invention pertains primarily to monoclonal antibodies (mAbs) that bind specifically to human C-C Motif Chemokine Receptor 8 (hCCR8), and related methods using such antibodies (Abs), including for detecting and quantifying the expression of hCCR8 on the surface of a cell, estimating receptor engagement by therapeutic anti-CCR8 mAbs, and measuring the depletion of CCR8-expressing regulatory T cells (Tregs) mediated by therapeutic anti-CCR8 mAbs. The invented mAbs disclosed for the first time herein (Abs of the invention) bind to an epitope that is distinct from epitopes located in the N-terminal region of CCR8 that have been shown to be bound by certain therapeutic anti-CCR8 mAbs; thus, the binding of any of the Abs of the invention to CCR8 is not affected by the presence an Ab bound to an N-terminal epitope.

### BACKGROUND OF THE INVENTION

Considerable success has been achieved in treating diverse solid tumors and hematological malignancies with immunotherapies that stimulate the activity of cytotoxic T cells by blocking immune checkpoint molecules, such as PD-1, PD-L1, CTLA-4 or LAG-3, which are known to suppress host antitumor immunity in the tumor microenvironment (Pardoll, 2012; Lesokhin et al., 2015; Baumeister et al., 2016; Pianko et al., 2017). However, typically less than around 15% of patients benefit long-term from treatment with a checkpoint inhibitor in cancers amenable to this treatment (Haslam and Prasad, 2019), and checkpoint inhibitors have proven to be less effective in certain cancers, including breast and prostate cancers. Thus, there is a pressing need for biomarkers that can be reliably used both to predict whether a particular cancer or patient is suitable for treatment with a particular immunotherapeutic, and for monitoring the mechanistic course of treatment at the molecular level.

The persistence of immunosuppressive mechanisms, especially those mediated by regulatory T cells (Tregs), may contribute to the observed resistance of certain cancers or certain patients to treatment with checkpoint inhibitors (Fares et al., 2019; Han et al., 2019). Accordingly, reducing the activity or numbers of tumor-infiltrating Tregs has been identified as an attractive approach to reversing immunosuppression and augmenting anti-tumor immunity (Finotello and Trajanoski, 2017; Han et al., 2019). It has recently been demonstrated that CCR8 expression is selectively upregulated in tumor-resident Tregs in multiple cancers (De Simone et al., 2016; Plitas et al., 2016), making CCR8 an attractive target to effect the depletion of tumor-resident Tregs in order to augment anti-tumor immunity.

PCT Publication No. WO 2021/194942 discloses several human or humanized mAbs that bind specifically with high affinity to hCCR8 expressed on a cell surface, mediate the depletion of CCR8⁺, tumor-infiltrating Tregs, and potently inhibit growth of tumors in a variety of mouse tumor models when administered to the mice as monotherapy or in combination with checkpoint blockade. One of these mAbs, A419, which is currently in Phase1/2 clinical trials (NCT04895709; https://clinicaltrials.gov/ct2/show/NCT04895709), was shown to bind to an epitope in the N-terminal region of hCCR8.

As disclosed herein, the majority of mAbs generated against cell surface-expressed hCCR8 immunogens bind to an N-terminal epitope. Several recent publications describing the generation of therapeutic anti-CCR8 Abs also describe Abs that bind to epitopes in the N-terminal domain, e.g., PCT Publication Nos. WO 2020/138489, WO 2021/142002, WO 2021/152186, WO 2021/163064, WO 2021/194942, WO 2021/260209, and WO 2022/136649. In contrast, the present invention relates to several mAbs that bind to an epitope other than the N-terminal epitope of mAb4A19 described in WO 2021/194942 and do not compete with mAb4A19 for binding to CCR8. The instant mAbs, referred to herein as “non-competing” mAbs by reference to their inability to compete with the therapeutic mAb4A19 for binding to hCCR8, can be used in many medicinal applications including to detect, measure the expression of, and measure receptor occupancy (RO) of, hCCR8 on the surface of a cell, and detect the depletion of a CCR8-expressing cell, even in the presence of a therapeutic anti-hCCR8 Ab bound to an N-terminal epitope. These properties of the mAbs disclosed herein make them useful for several purposes, including measuring the depletion of CCR8-expressing Tregs mediated by an anti-CCR8 therapeutic Ab, and various diagnostic and biomarker applications.

### SUMMARY OF THE INVENTION

The present disclosure provides isolated Abs of the invention, preferably mAbs, that specifically bind to CCR8, such as human CCR8 (hCCR8), expressed on the surface of a cell and exhibit various functional properties, including properties that are desirable in a diagnostic Ab that can be used to measure CCR8 expression in patients being treated with a therapeutic anti-CCR8 Ab. These properties include binding with high affinity and specificity to CCR8-expressing cells, such as tumor-infiltrating, activated CD4⁺FOXP3^{high} Tregs, and binding to a hCCR8 epitope that is distinct from an epitope in the N-terminal domain of hCCR8 to which therapeutic anti-CCR8 Abs such as mAb4A19 bind.

Specifically, this disclosure provides mAbs, or antigen-binding portions thereof, that bind specifically to hCCR8 expressed on the surface of a cell, wherein the mAbs or antigen-binding portions thereof binds to an epitope other than an epitope in the N-terminal domain of hCCR8. MAb 4A19 (WO 2021/194942) was shown to bind to an N-terminal epitope comprising at least one amino acid, and in preferred embodiments, all 11 of the amino acids, within a peptide having the sequence V₁₂T₁₃D₁₄Y₁₅Y₁₆Y₁₇P₁₈D₁₉I₂₀F₂₁S₂₂ (SEQ ID NO: 73). In certain embodiments, the N-terminal epitope to which the anti-hCCR8 mAbs of the present disclosure do not bind comprises at least one amino acid within a peptide having the sequence V₁₂T₁₃D₁₄Y₁₅Y₁₆Y₁₇P₁₈D₁₉I₂₀F₂₁S₂₂ (SEQ ID NO: 73), e.g., comprising 1, 2, 3, 4, 5, 6, 7, 8, 9, 10 or all 11 of the amino acids within a peptide having the sequence set forth in SEQ ID NO: 73. In certain preferred embodiments, the N-terminal epitope to which the anti-hCCR8 mAbs of the present disclosure do not bind comprises a peptide having the sequence Y₁₅Y₁₆Y₁₇P₁₈D₁₉I₂₀F₂₁ (SEQ ID NO: 2). In other preferred embodiments, the N-terminal epitope to which the anti-hCCR8 mAbs of the present disclosure do not bind comprises a peptide having the sequence V₁₂T₁₃D₁₄Y₁₅Y₁₆Y₁₇P₁₈D₁₉I₂₀F₂₁S₂₂ (SEQ ID NO: 73). In other preferred embodiments, the N-terminal epitope to which the anti-hCCR8 mAbs of the present disclosure do not bind consists of a peptide having the sequence V₁₂T₁₃D₁₄Y₁₅Y₁₆Y₁₇P₁₈D₁₉I₂₀F₂₁S₂₂ (SEQ ID NO: 73).

In certain preferred embodiments, the binding of an Ab of the invention or an antigen-binding portion thereof to hCCR8 is not affected by the presence of an Ab bound to an N-terminal epitope, such as for example, an epitope comprising at least one amino acid, such as comprising 1, 2, 3, 4, 5, 6, 7, 8, 9, 10 or all 11 of the amino acids, within a peptide having the sequence V₁₂T₁₃D₁₄Y₁₅Y₁₆Y₁₇P₁₈D₁₉I₂₀F₂₁S₂₂ (SEQ ID NO: 73). In certain embodiments, amino acid Y₁₅ or Y₁₇, and preferably both Y₁₅ and Y₁₇, within the N-terminal epitope are sulfated. In further embodiments, the Ab bound to the N-terminal epitope is a mAb comprising the 6 CDRs (SEQ ID NOs. 53-58), heavy and/or light variable regions (SEQ ID NO: 9 and/or 16), or heavy and/or light chains (SEQ ID NO: 65 and/or 72) of mAb 4A19 described in WO 2021/194942. In further embodiments, the Ab bound to the N-terminal epitope is mAb 4A19 which comprises the heavy and/or light chains (SEQ ID NO: 65 and/or 72) of mAb 4A19.

In certain preferred embodiments, the mAb or antigen-binding portion thereof that binds to an epitope other than the N-terminal epitope is a mAb or antigen-binding portion thereof comprising the 6 CDRs, heavy and/or light variable regions, or heavy and/or light chains of mAbs designated herein as 25T40, 21C17, 28P3, 22B13, 33H18 or 23A14.

The disclosed invention also provides a labeled Ab, or an antigen-binding thereof, comprising a mAb of the invention and a detectable label. In different embodiments, the detectable label is a fluorophore, a chromophore, an enzyme, a radioactive isotope, a micropolymer, or a metal.

The disclosure further provides a method for generating a first Ab (e.g., an Ab of the invention), that does not bind, or does not cross-compete with a second Ab (e.g., anti-hCCR8 Clone L263G8 commercialized by BioLegend, mAb 433H marketed by BD Biosciences, or any of mAbs 4A19, 18Y12, 8D55, 10R3, 14S15 and 14S15h described in WO 2021/194942) for binding, to a defined epitope on an antigen (e.g., an N-terminal epitope of hCCR8). The method comprises immunizing a vertebrate with an immunogen comprising a cell line, or a component of said cell line, that expresses the antigen and also expresses the second Ab or antigen-binding portion thereof that binds specifically to the epitope, wherein the binding of the second Ab or antigen-binding portion thereof to the epitope shields said epitope from the immune system of the vertebrate, reducing the generation of Abs that bind to the epitope and thereby resulting in the generation of a first Ab that does not bind, or does not cross-compete with the second Ab for binding, to the epitope.

This disclosure also provides a method for measuring a depletion in the number of Tregs in a subject comprising: (a) determining a baseline percentage of T cells that are CCR8-expressing Tregs in a first test tissue in or taken from a subject, the test tissue comprising tumor cells and tumor-infiltrating Tregs; (b) administering a treatment to the subject; and (c) determining the percentage of T cells that are CCR8-expressing Tregs in a second test tissue in or taken from a subject during or after the treatment; wherein a decrease in the percentage of T cells that are CCR8-expressing Tregs in the second test tissue indicates that the number of Tregs in the test tissue has been depleted. In certain preferred embodiments, the treatment administered to the subject is a treatment for cancer. In further preferred embodiments, the treatment for cancer comprises administering a Treg-depleting therapeutic anti-CCR8 Ab or an antigen-binding portion thereof to the subject as monotherapy or in combination with another anti-cancer therapy.

Also provided by this disclosure is a method for predicting the effectiveness of a therapeutic Treg-depleting anti-CCR8 Ab or an antigen-binding portion thereof in treating cancer in a subject, which method comprises: (a) determining the percentage of T cells that are CCR8-expressing Tregs in a test tissue in or taken from a subject, the test tissue comprising tumor cells and tumor-infiltrating Tregs; (b) comparing the percentage of T cells that are CCR8-expressing Tregs with a predetermined threshold value; and (c) predicting the effectiveness of the therapeutic anti-CCR8 Ab, wherein a percentage of T cells that are CCR8-expressing Tregs exceeding the threshold value indicates that the therapeutic Ab or antigen-binding portion thereof will be effective in treating the subject, and further wherein a percentage of T cells that are CCR8-expressing Tregs less than the threshold value indicates that the therapeutic Ab or antigen-binding portion thereof will not be effective in treating the subject.

The invention also relates to methods for treating cancer in a patient. Accordingly, the present disclosure provides a method for treating a cancer in a subject, which method comprises: (a) selecting a subject that is a suitable candidate for immunotherapy with a Treg-depleting therapeutic anti-CCR8 Ab or an antigen-binding portion thereof, the selecting comprising: (i) determining the percentage of T cells that are CCR8-expressing Tregs in a test tissue in or taken from the subject, the test tissue comprising tumor cells and tumor-infiltrating Tregs; (ii) comparing the percentage of T cells that are CCR8-expressing Tregs with a predetermined threshold value; and (iii) selecting the subject as a suitable candidate for immunotherapy with the therapeutic anti-CCR8 Ab or antigen-binding portion thereof based on an assessment that the percentage of T cells that are CCR8-expressing Tregs in cells of the test tissue exceeds the predetermined threshold value; and (b) administering to the selected subject a composition comprising a therapeutically effective amount of the therapeutic anti-CCR8 Ab or antigen-binding portion thereof.

The disclosure also provides a method for treating a cancer in a subject, which method comprises: (a) selecting a subject that is not a suitable candidate for immunotherapy with a Treg-depleting therapeutic anti-CCR8 Ab or an antigen-binding portion thereof, the selecting comprising: (i) determining the percentage of T cells that are CCR8-expressing Tregs in a test tissue in or taken from the subject, the test tissue comprising tumor cells and tumor-infiltrating Tregs; (ii) comparing the percentage of T cells that are CCR8-expressing Tregs with a predetermined threshold value; and (iii) selecting the subject as not suitable for immunotherapy with the therapeutic anti-CCR8 Ab or an antigen-binding portion thereof based on an assessment that the percentage of T cells that are CCR8-expressing Tregs in cells of the test tissue is less than the predetermined threshold value; and (b) administering to the selected subject a standard-of-care therapeutic other than a therapeutic anti-CCR8 Ab or an antigen-binding portion thereof.

This disclosure further provides a method for treating cancer in a subject, which method comprises administering to the subject a composition comprising a therapeutically effective amount of a Treg-depleting therapeutic anti-CCR8 Ab or an antigen-binding portion thereof, the subject having been selected on the basis that the percentage of T cells that are CCR8-expressing Tregs in cells of a test tissue in or taken from the subject, the test tissue comprising tumor cells and tumor-infiltrating Tregs, was determined to exceed a predetermined threshold level.

The disclosure still further provides a method for treating cancer in a subject, which method comprises administering to the subject a standard-of-care treatment other than a Treg-depleting therapeutic anti-CCR8 Ab or an antigen-binding portion thereof, the subject having been selected on the basis that the percentage of T cells that are CCR8-expressing Tregs in cells of a test tissue in or taken from the subject, the test tissue comprising tumor cells and tumor-infiltrating Tregs, was determined to be less than a predetermined threshold level.

In certain embodiments of any of these therapeutic methods for treating a cancer, the Treg-depleting therapeutic anti-CCR8 Ab or antigen-binding portion thereof comprises the 6 CDRs, heavy and/or light variable regions, or heavy and/or light chains of mAb 4A19 described in WO 2021/194942 as binding specifically and with high affinity to CCR8 expressed on a cell surface and mediating the depletion of the cell, or an antigen-binding portion thereof. In further embodiments, the Treg-depleting therapeutic anti-CCR8 Ab or antigen-binding portion thereof is the mAb designated 4A19, 18Y12, 10R3, 8D55, 14S15, or 14S15h, or an antigen-binding portion thereof. These Abs, disclosed in WO 2021/194942, bind to N-terminal epitopes of hCCR8.

In yet other embodiments, the therapeutic method further comprises administering to the subject a therapeutically effective amount of an additional therapy for treating the cancer. Such an additional anti-cancer therapy may be a small molecule agent, a polypeptide, an antibody, an immunoregulatory agent, a chemotherapy, a targeted therapy, a radiation therapy, a surgery, or any combination thereof. In certain embodiments, the immunotherapy comprises an agent that reduces inhibition, or increases stimulation, of the immune system. In certain preferred embodiments, the immunoregulatory agent that reduces inhibition of the immune system is an immune checkpoint inhibitor. In further preferred embodiments, the immunoregulatory agent that reduces inhibition of the immune system is an immune checkpoint inhibitor such as an antagonist of PD-1, PD-L1, CTLA-4, LAG-3, TIGIT and/or TIM-3. In certain embodiments, the chemotherapy comprises an alkylating agent such as dacarbazine, ifosfamide, cyclophosphamide or the platinum-based chemotherapeutics cisplatin, bendamustine, carboplatin, and oxaliplatin; a mitotic inhibitor such as the vinca alkaloids vinblastine and vincristine, or the taxanes docetaxel, paclitaxel and cabazitaxel; a topoisomerase inhibitor such as etoposide or irinotecan; an antimetabolite such as 5-fluorouracil, azacytidine or gemcitabine; or an antitumor antibiotic such as bleomycin, mitomycin-C, or the anthracyclines daunorubicin, doxorubicin, and mitoxantrone. In certain preferred embodiments, the additional anti-cancer therapy is an anti-PD-1 antibody or docetaxel.

The disclosure also provides a variety of kits for performing the methods described herein, including kits for use in methods of: measuring receptor (e.g., CCR8) occupancy by a Treg-depleting therapeutic Ab (e.g., an anti-CCR8, Treg-depleting therapeutic Ab); measuring a depletion in the number of Tregs in a subject; predicting the effectiveness of a therapeutic anti-CCR8 Ab; selecting a subject afflicted with a cancer as a suitable candidate for immunotherapy with the therapeutic anti-CCR8 Ab; and treating cancer in a subject. By way of example, the disclosure provides a kit for use in measuring a depletion in the number of Tregs in a subject, the kit comprising: (a) a mAb or an antigen-binding portion thereof that binds specifically to hCCR8 expressed on the surface of a cell, wherein the mAb or antigen-binding portion thereof binds to an epitope other than an epitope in the N-terminal domain of hCCR8; and (b) instructions for using the mAb or portion thereof in any one of the methods of measuring Treg depletion disclosed herein.

By way of another example, the disclosure provides a kit for use in treating cancer in a subject, the kit comprising: (a) a mAb or an antigen-binding portion thereof that binds specifically to hCCR8 expressed on the surface of a cell, wherein the mAb or antigen-binding portion thereof binds to an epitope other than an epitope in the N-terminal domain of hCCR8; (b) a Treg-depleting therapeutic anti-CCR8 Ab or an antigen-binding portion thereof, and (c) instructions for using the mAb or portion thereof and the therapeutic anti-CCR8 Ab or antigen-binding portion thereof in any one of the methods of treating cancer in a subject disclosed herein.

Other features and advantages of the instant invention will be apparent from the following detailed description and examples which should not be construed as limiting. The contents of all cited references, including scientific articles, GenBank entries, patents and patent applications cited throughout this application are expressly incorporated herein by reference.

### BRIEF DESCRIPTION OF THE FIGURES

FIGS. 1A-1C: Flow cytometric plots showing binding of two test mAbs, BV421-labeled 21C17 on the x-axis and phycoerythrin (PE)-labeled Clone L263G8 (BioLegend) on the y-axis, to hCCR8 from a gastric tumor Treg compartment pretreated with unlabeled mAbs 4A19 (FIG. 1A), 21C17 (FIG. 1B) or an isotype control (FIG. 1C). Binding to 4A19-pretreated Tregs was observed with the diagnostic BV421-labeled 21C17 mAb (FIG. 1A, Quadrant 3), whereas the commercial PE-labeled L263G8 mAb did not bind to 4A19-pretreated Tregs (FIG. 1A). Binding to 21C17-pretreated Tregs was observed with PE-L263G8 Ab (FIG. 1B, Q1), whereas BV421-21C17 did not bind to 21C17-pretreated Tregs (FIG. 1B), demonstrating 21C17 self-blocking. Both labeled 4A19 and 21C17 mAbs bound to the same Tregs pretreated with the isotype control Ab (FIG. 1C, Q2). These results indicate that mAb 21C17 is able to bind to CCR8-expressing tumor Tregs that have been pretreated with the 4A19 mAb.

FIGS. 2A and 2B show the binding of 6 mAbs of the invention (25T40, 21C17, 28P3, 22B13, 33H18 and 23A14) specifically to hCCR8-overexpressing Raji cells (FIG. 2A) versus parental Raji cells that do not express CCR8 (FIG. 2B), as evaluated by FACS using fluorescently labeled secondary Abs. Four of the 6 mAbs bound to the hCCR8-expressing cells with an EC₅₀ below 1 nM (FIG. 2A; see Table 1) whereas no nonspecific binding was observed on the parental Raji cells (FIG. 2B).

FIGS. 3A-3C show an evaluation of competition between Abs of the invention and an Ab that binds to an N-terminal epitope for binding to hCCR8. A: A mouse IgG2a variant of mAb 4A19 (4A19-mG2a) was bound to activated human Tregs at a saturating concentration of 200 nM, and 23A14-hG1 mAb was subsequently added at concentrations ranging from 200 nM to 0.0034 nM. Analysis of the bound 4A19-mG2a and 23A14-hG1, measured by FACS using fluorescently labeled secondary Abs (FIG. 3A), shows that at saturating concentrations both 4A19-mG2a and 23A14-hG1 were able to bind simultaneously to equally high percentages of CCR8⁺ Tregs. B, C: Activated human Tregs were incubated with mAb 4A19 at titrated concentrations ranging from 200 nM to 0.0034 nM, followed by incubation with 100 nM of 21C17-mG2a (FIG. 3B) or 22B13-mG2a (FIG. 3C). The FACS competition assay analysis using fluorescently labeled secondary Abs shows that 4A19, when paired with either 21C17-mG2a or 22B13-mG2a at saturating concentrations, is able to bind simultaneously to equally high percentages of CCR8⁺ Tregs.

FIGS. 4A and 4B show the binding of mAb 21C17-mG2a, directly conjugated to a BV421 fluorophore, specifically to hCCR8-overexpressing Raji cells (FIG. 4A) versus parental Raji cells that do not express CCR8 (FIG. 4B), as evaluated by FACS. Raji parental cells and CCR8-overexpressing Raji cells were incubated with titrations of unlabeled 21C17-mG2a and either BV421-labeled 21C17-mG2a or Keyhole limpet hemocyanin (KLH)-mg2a control Abs. Binding of the unlabeled 21C17-mG2a mAb to cell surface hCCR8 was detected using phycoerythrin (PE)-labeled anti-mouse IgG secondary Ab. The relative cell binding was measured as the geometric mean fluorescence intensity (GMFI) of total cells positive for fluorescently conjugated secondary Ab and fluorescence from directly conjugated Ab. The labeled and unlabeled parental 21C17-mG2a Abs bind specifically and with similar efficiency to cell surface CCR8 on the overexpressing Raji cell line (FIG. 4A) but do not bind to the Raji parental cell line (FIG. 4B).

FIG. 5 shows a representative figure demonstrating CCR8 percentage receptor occupancy (% RO) response curves, with CCR8% RO plotted against mAb 4A19 concentration in healthy donor blood (n=3). The % RO curves were plotted using the direct vs. indirect RO assay format for each donor. Response curves from assays on blood from donors ##1199, 0612 and 398 are shown.

FIG. 6 shows the depletion of Tregs in dissociated human tumors by anti-hCCR8 mAb, 4A19. Following 48 h of treatment of dissociated tumor tissues with mAb 4A19 or an anti-KLH isotype Ab control, cells were stained with a viability stain to identify live cells, then stained with the non-competing anti-CCR8 mAb 21C17, along with conjugated CD4, FoxP3, and CD25 Abs to identify CCR8⁺ Tregs. MAb 4A19 depleted Tregs, but the KLH isotype control did not, even at the highest dose tested.

### DETAILED DESCRIPTION OF THE INVENTION

The present invention relates to mAbs that do not bind specifically to an epitope in the N-terminal of hCCR8, and to uses of such mAbs. That is, the invention relates to mAbs that bind specifically to an epitope of hCCR8 that differs from an epitope in the N-terminal domain to which Treg-depleting therapeutic Abs such as mAb 4A19 bind, and to methods, for example, for detecting and/or measuring the level of expression of hCCR8 on the surface of a cell using such mAbs. MAbs that bind to this epitope of hCCR8 outside of the N-terminal domain do not compete with mAbs that bind to the N-terminal epitope and, thus, the binding of the former mAbs to CCR8 is not affected by the presence of Abs bound to the N-terminal domain. Thus, the mAbs of the invention can be used to detect and/or measure the level of expression of hCCR8 on the surface of a cell even in the presence of a therapeutic mAb bound to the N-terminal epitope. This feature is very useful in use of the mAbs of the invention for diagnostic or biomarker applications.

### Terms

In order that the present disclosure may be more readily understood, certain terms are first defined. As used in this application, except as otherwise expressly provided herein, each of the following terms shall have the meaning set forth below. Additional definitions are set forth throughout the application.

“Administering”, “administer” or “administration” refers to the physical introduction of a composition comprising an agent, e.g., a therapeutic or diagnostic agent, to a subject, using any of the various methods and delivery systems known to those skilled in the art. A preferred route for administration of a therapeutic or diagnostic Ab such as an anti-CCR8 Ab is intravenous (IV) administration. Other routes of administration include subcutaneous (SC), intraperitoneal (IP), intramuscular (IM), spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, an Ab of the invention can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods.

An “antibody” (Ab) shall include, without limitation, a glycoprotein immunoglobulin (Ig) which binds specifically to an antigen and comprises at least two heavy (H) chains and two light (L) chains interconnected by disulfide bonds, or an antigen-binding portion thereof. Each H chain comprises a heavy chain variable region (abbreviated herein as V_{H}) and a heavy chain constant region. The heavy chain constant region of an IgG Ab comprises three constant domains, C_{H1}, C_{H2} and C_{H3}. Each light chain comprises a light chain variable region (abbreviated herein as V_{L}) and a light chain constant region. The light chain constant region of an IgG Ab comprises one constant domain, C_{L}. The V_{H} and V_{L} regions can be further subdivided into regions of hypervariability, termed complementarity determining regions (CDRs), interspersed with regions that are more conserved, termed framework regions (FR). Each V_{H} and V_{L} comprises three CDRs and four FRs, arranged from amino-terminus to carboxy-terminus in the following order: FR1-CDR1-FR2-CDR2-FR3-CDR3-FR4. The variable regions of the heavy and light chains contain a binding domain that interacts with an antigen. A variety of methods have been used to delineate the CDR domains within an Ab, including the Kabat, Chothia, AbM, contact, and IMGT definitions. The constant regions of the Abs may mediate the binding of the Ig to host tissues or factors, including various cells of the immune system (e.g., effector cells) and the first component (C1q) of the classical complement system.

As used herein, and in accordance with conventional usage, an Ab that is described as comprising “a” heavy chain and/or “a” light chain refers to an Ab that comprise “at least one” of the recited heavy and/or light chains, and thus will encompass Abs having two or more heavy and/or light chains. Specifically, Abs so described will encompass conventional Abs having two substantially identical heavy chains and two substantially identical light chains. Ab chains may be substantially identical but not entirely identical if they differ due to post-translational modifications, including, for example, C-terminal cleavage of lysine residues, and alternative glycosylation patterns.

An Ig may derive from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the Ab class or subclass (e.g., IgM, IgG1, or IgG4) that is encoded by the heavy chain constant region genes. The term “antibody” includes, by way of example, both naturally occurring and non-naturally occurring Abs, monoclonal and polyclonal Abs, chimeric and humanized Abs, human or nonhuman Abs, wholly synthetic Abs, and single chain Abs. A nonhuman Ab may be humanized partially or fully by recombinant methods to reduce its immunogenicity in man. Where not expressly stated, and unless the context indicates otherwise, the term “antibody” also includes an antigen-binding fragment or an antigen-binding portion of any of the aforementioned Ig's, and includes a monovalent and a divalent fragment or portion, and a single chain Ab.

An “isolated” Ab refers to an Ab that is substantially free of other Abs having different antigenic specificities (e.g., an isolated Ab that binds specifically to CCR8 is substantially free of Abs that bind specifically to antigens other than CCR8, such as Abs that bind to CCR4). An isolated Ab that binds specifically to human CCR8 (hCCR8) may, however, have cross-reactivity to other antigens, such as CCR8 polypeptides from different species such as mouse and cynomolgus monkey. Moreover, in certain contexts, an isolated Ab may also mean an Ab that is purified so as to be substantially free of other cellular material and/or chemicals. By comparison, an “isolated” nucleic acid refers to a nucleic acid composition of matter that is markedly different, i.e., has a distinctive chemical identity, nature and utility, from nucleic acids as they exist in nature. For example, an isolated DNA, unlike native DNA, is a free-standing portion of a native DNA and not an integral part of a larger structural complex, the chromosome, found in nature. Further, an isolated DNA, unlike native DNA, can be used as a PCR primer or a hybridization probe for, among other things, measuring gene expression and detecting biomarker genes or mutations for diagnosing disease or predicting the efficacy of a therapeutic. In addition, in certain contexts, an isolated nucleic acid may mean a nucleic acid that is purified so as to be substantially free of other cellular components or other contaminants, e.g., other cellular nucleic acids or proteins, using standard techniques well known in the art.

The term “monoclonal” Ab (mAb) refers to a non-naturally occurring preparation of Ab molecules of single molecular composition, i.e., Ab molecules whose primary sequences are essentially identical and which exhibit a single binding specificity and affinity for a particular epitope. A mAb is an example of an isolated Ab. MAbs may be produced by hybridoma, recombinant, transgenic or other techniques known to those skilled in the art.

A “chimeric” Ab refers to an Ab in which the variable regions are derived from one species and the constant regions are derived from another species, such as an Ab in which the variable regions are derived from a mouse Ab and the constant regions are derived from a human Ab.

A “human” mAb (HuMAb) refers to a mAb having variable regions in which both the framework and CDR regions are derived from human germline immunoglobulin sequences. Furthermore, if the Ab contains a constant region, the constant region also is derived from human germline immunoglobulin sequences. The human Abs of the invention may include amino acid residues not encoded by human germline immunoglobulin sequences (e.g., mutations introduced by random or site-specific mutagenesis in vitro or by somatic mutation in vivo). However, the term “human” Ab, as used herein, is not intended to include Abs in which CDR sequences derived from the germline of another mammalian species, such as a mouse, have been grafted onto human framework sequences. The terms “human” Abs and “fully human” Abs are used synonymously.

A “humanized” mAb refers to a mAb in which some, most or all of the amino acids outside the CDR domains of a non-human mAb are replaced with corresponding amino acids derived from human immunoglobulins. In one embodiment of a humanized form of an Ab, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the Ab to bind to a particular antigen. A “humanized” Ab retains an antigenic specificity similar to that of the original Ab.

An “anti-antigen” Ab refers to an Ab that binds specifically to an antigen. For example, an anti-CCR8 Ab is an Ab that binds specifically to CCR8.

An “antigen-binding portion” or “antigen-binding fragment” of an Ab refers to one or more fragments of an Ab, e.g., a mAb, that retain the ability to bind specifically to the antigen bound by the whole Ab. An anti-CCR8 antigen-binding portion or fragment that mediates depletion of a CCR8-expressing cell by, for example, Ab-dependent cellular cytotoxicity (ADCC), Ab-dependent cell-mediated phagocytosis (ADCP), or complement-dependent cytotoxicity (CDC) necessarily comprises a portion of the Fc region of the Ab required to mediate these effector functions via its interaction with Fc-receptors on immune cells or with C1q.

“Antibody-dependent cell-mediated cytotoxicity” (“ADCC”) refers to an in vitro or in vivo cell-mediated cytotoxic activity in which nonspecific effector cells that express Fc receptors (FcRs) on the effector cell surface (e.g., natural killer (NK) cells, macrophages, neutrophils and eosinophils) recognize the Fc region of Abs bound to surface antigens on a target cell and actively lyses the target cell. In principle, any effector cell with an activating FcR can be triggered to mediate ADCC.

Antibody-dependent cell-mediated phagocytosis (“ADCP”) is an immunological mechanism of eliminating cells whereby phagocytic immune cells, such as monocytes, macrophages, and neutrophils, that express Fc receptors (FcRs) on the cell surface, recognize the Fc region of Abs bound to surface antigens on a target cell to induce phagocytosis, resulting in internalization and degradation of the target cell through phagosome acidification.

A “cancer” refers a broad group of various diseases characterized by the uncontrolled growth of abnormal cells in the body. Unregulated cell division and growth divide and grow results in the formation of malignant tumors that invade neighboring tissues and may also metastasize to distant parts of the body through the lymphatic system or bloodstream.

“C-C Motif Chemokine Receptor 8” (“CCR8”; also known in the art as, for example, CY6, TER1, CCR-8, CKRL1, CDw198, CMKBR8, GPRCY6, CMKBRL2, or CC-CKR-8) is a G-protein-coupled seven-transmembrane chemokine receptor (GPCR) expressed primarily on intratumoral FOXP3^{hi} Tregs. The term “CCR8” as used herein includes human CCR8 (hCCR8), variants, isoforms, species homologs of hCCR8 such as mouse CCR8 (mCCR8), and analogs having at least one common epitope with hCCR8. The complete hCCR8 and mCCR8 amino acid sequences can be found under GENBANK® Accession Nos. AAI07160.1 and NP_031746.1, respectively.

A “cell surface receptor” refers to molecules or complexes of molecules expressed on the surface of a cell that are capable of receiving a signal and transmitting the signal across the plasma membrane of the cell.

Complement-dependent cytotoxicity (“CDC”) is an immune response in which target cells are lysed through activation and recruitment of the complement cascade to the targeted cell surface. It is an effector function of IgG (mainly IgG1 and IgG3) and IgM Abs. The binding of C1q to these Abs, when bound to surface antigen on target cell, triggers the activation of the classical complement pathway, resulting in formation of a membrane attack complex (MAC) and target cell lysis.

An “immune response” refers to a biological response within a vertebrate against foreign agents, which response protects the organism against these agents and diseases caused by them. The immune response is mediated by the action of one or more cells of the immune system (for example, a T lymphocyte, B lymphocyte, natural killer (NK) cell, macrophage, eosinophil, mast cell, dendritic cell or neutrophil) and soluble macromolecules produced by any of these cells or the liver (including Abs, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues.

The term “immunotherapy” refers to the treatment of a disease in a subject, or treatment of a subject at risk of contracting or suffering a recurrence of, the disease by a method comprising inducing, enhancing, suppressing, or otherwise modifying an immune response. “Cancer immunotherapy” refers to the application of immunotherapy to the treatment or prevention of cancer, usually by inducing or enhancing the immune response, such as by blocking immunosuppressive pathways or mechanisms in a subject.

The term “positron emission tomography” or “PET” refers to a non-invasive imaging technique that uses radioactive substances to visualize molecular targets and measure metabolic processes in the body of a subject. The technique detects pairs of gamma rays emitted indirectly by a positron-emitting radionuclide (tracer) introduced into the body on a biologically active molecule and produces a three-dimensional image of tracer location in the body. Exemplary applications of PET imaging tools in drug development include direct visualization of and changes in quantity of targets, monitoring drug uptake and metabolism in different tissues to anticipate toxicity or patient to patient variation, quantifying diseased tissue, evaluating tumor metastasis, and monitoring drug efficacy or resistance over time.

A “subject” includes any human or nonhuman animal. The term “nonhuman animal” includes, but is not limited to, vertebrates such as nonhuman primates, sheep, dogs, and rodents such as mice, rats and guinea pigs. In preferred embodiments, the subject is a human. The terms “subject” and “patient” are used interchangeably herein.

A “therapeutically effective amount” or “therapeutically effective dosage” of a drug or therapeutic agent is any amount of the drug or agent that, when used alone or in combination with another therapeutic agent, protects a subject against the onset of a disease or promotes disease regression evidenced by a decrease in severity of disease symptoms, an increase in frequency and duration of disease symptom-free periods, a prevention or reduction of impairment or disability due to the disease affliction, or otherwise an amelioration of disease symptoms in the subject.

“Treatment” or “therapy” of a subject refers to any type of intervention or process performed on, including the administration of an active agent to, the subject with the objective of reversing, alleviating, ameliorating, inhibiting, slowing down or preventing the onset, progression, development, severity or recurrence of a symptom, complication or condition, or biochemical indicia associated with a disease.

As used herein, the indefinite articles “a” or “an” should be understood to refer to “one or more” of any recited or enumerated component.

The term “about”, when applied to a numeric value, refers to a value that is reasonably close to the stated value and within an acceptable error range as determined by one of ordinary skill in the art, which will depend in part on how the value is measured or determined, e.g., on the limitations of the measurement system. By way of example, “about” can mean a range of plus or minus 50% of a stated reference value, preferably a range of plus or minus 25%, or more preferably a range of plus or minus 10%. These ranges typically fall within an acceptable error range for that particular value according to the practice in the art.

The term “substantially the same” or “essentially the same” refers to a sufficiently high degree of similarity between two or more numeric values, substances, compositions of matter, or characteristics, that one of skill in the art would consider the difference between these values, substances, compositions of matter, or characteristics, to be of little or no biological and/or statistical significance within the context of the property being measured. The difference between numeric values being measured may, for example, be less than about 50%, preferably less than about 25%, and more preferably less than about 10%.

As described herein, any concentration range, percentage range, ratio range or integer range is to be understood to include the value of any integer within the recited range and, when appropriate, fractions thereof (such as one tenth and one hundredth of an integer), unless otherwise indicated.

Various aspects of the invention are described in further detail in the following subsections.

### Targeting CCR8 Due to its Expression Specifically on Tumor-Infiltrating Tregs

CCR8 expression has been shown to be selectively upregulated in tumor-resident Tregs in multiple cancers (De Simone et al., 2016; Plitas et al., 2016) and expressed on the most activated and suppressive subset of FOXP3^{hi} tumor Tregs associated with poor survival (Plitas et al., 2016; Wang et al., 2019; WO 2021/194942). Human CCR8 and FOXP3 gene-gene correlations in The Cancer Genome Atlas (TCGA) has further shown that CCR8 expression has the highest correlation with FOXP3 (master transcriptional regulator of Tregs) in most cancer types, with CCR8 expressed on tumor FOXP3^{hi} Tregs, but rarely observed on Tregs and Teffs in the peripheral blood (WO 2021/194942). CCR8 is also selectively expressed on: FOXP3^{hi} lymphocytes in hepatocellular carcinoma tumor samples but not in FOXP3^{mid} and FOXP3^{neg} CD8 and CD4 effector T cells in patient tumors; a high proportion of tumor-resident Tregs but a much lower proportion of tumor-infiltrating CD4⁺ T cells and CD8⁺ T cells; and a small fraction of peripheral Tregs but on the majority of tumor-infiltrating Tregs (WO 2021/194942).

These patterns of CCR8 expression make CCR8 a highly desirable target for mediating depletion of these highly immunosuppressive Tregs through ADCC and ADCP using an anti-CCR8 Ab, and because CCR8 is rarely expressed on Tregs and Teffs in peripheral blood or in other tissues, targeting Tregs poses minimal toxicity risks. WO 2021/194942 describes the generation and characterization of multiple anti-hCCR8 mAbs that exhibit properties desirable in a therapeutic Ab for treating cancer, including a high efficiency in mediating depletion of CCR8-expressing, tumor-associated Tregs. These Abs bind to an epitope in the extracellular N-terminal domain of hCCR8, creating a need for Abs that bind to an epitope outside the N-terminal domain. Such Abs, that do not affect or are not affected by the binding of a therapeutic Ab to the N-terminal domain of CCR8, are useful for diagnostic applications involving the measurement or monitoring of CCR8 expression on Tregs, and the numbers of CCR8-expressing Tregs, even in the presence of a therapeutic Ab bound to the N-terminus of CCR8.

Generation of Anti-hCCR8 MAbs that Do Not Bind to N-Terminal Domain MAbs were generated by immunizing mice with immunogens comprising plasma membrane materials derived from hCCR8-overexpressing cells. Because most anti-hCCR8 Abs had been found to bind to one or more epitopes in the N-terminal domain of hCCR8, a strategy was developed to preferentially generate Abs that bind to an epitope different from these N-terminal epitopes. This strategy (see Example 1) involved using as the immunogen proteoliposomes derived from cells that overexpressed hCCR8 and also expressed an anti-hCCR8 Ab, Clone L263G8 (BioLegend), which binds to an N-terminal epitope. It was expected that this expressed Ab would bind to an N-terminal epitope of CCR8. Therefore, upon using the proteoliposomes prepared from the cells as immunogens, the N-terminal epitope(s) would be shielded by the bound Ab from the immune system of the mouse, and Abs generated would be preferentially directed to epitopes other than N-terminal epitopes.

This strategy was successfully demonstrated to generate mouse Abs that bind to an epitope on hCCR8 that is different from an N-terminal epitope. Specifically, as described in Example 1, this method was used to generate Abs that bind to an epitope outside the N-terminal domain of hCCR8 by immunizing mice with an immunogen comprising proteoliposome material derived from cells overexpressing both a chimeric hCCR8/hCCR5 protein and an anti-hCCR8 Ab (Clone L263G8) that binds to an epitope in the N-terminal domain of hCCR8. However, this approach is broadly applicable to generating Abs that do not bind to a predefined epitope of an antigen.

Accordingly, this disclosure describes a method for generating a first Ab that does not bind, or does not cross-compete with a second Ab for binding, to a defined epitope on an antigen, the method comprising immunizing a vertebrate with an immunogen comprising a cell line, or a component of said cell line, that expresses the antigen and also expresses the second Ab or antigen-binding portion thereof, wherein the second Ab binds specifically to the epitope, wherein the binding of the second Ab or antigen-binding portion thereof to the epitope shields said epitope from the immune system of the vertebrate, reducing the generation of Abs that bind to the epitope and thereby preferentially resulting in the generation of a first Ab that does not bind, or does not cross-compete with the second Ab for binding, to the epitope.

In certain embodiments, the vertebrate is a mouse, as demonstrated in Example 1; or another mammal such as a rat, hamster, rabbit, dog, goat, sheep, or horse; or a bird such as a chicken. In certain embodiments, the antigen is a CCR8 receptor, such as a human, cynomolgus monkey, mouse, or rat CCR8 receptor. In preferred embodiments, the antigen is a hCCR8 receptor. In certain embodiments of the method for generating Abs against hCCR8, the epitope is an epitope in the N-terminal domain of the hCCR8 receptor. In further embodiments, the second Ab or antigen-binding portion thereof is the mAb designated Clone L263G8 (BioLegend); the mAb designated 433H (BD Biosciences) or mAb 4A19, 18Y12, 10R3, 8D55, 14S15, or 15S15h, as described in WO 2021/194942. In certain other embodiments, the immunogen is a detergent-stabilized proteoliposome component of the cell line.

MAbs of the Invention that do not Bind to an N-Terminal Epitope of hCCR8

Using multiple immunization campaigns to generate anti-hCCR8 mAbs, including the method described above to shield the N-terminal epitope(s) from the immune system of a mouse, mouse Abs were generated that bind to an epitope on hCCR8 other than the N-terminal epitope(s). The immunogen was detergent-stabilized proteoliposome material derived from HEK293 cells engineered to overexpress hCCR8 as well as the anti-CCR8 mAb designated Clone L263G8 (BioLegend) which binds to an N-terminal epitope of hCCR8. Ab-secreting B cells from immunized mice were fused with immortalized myeloma cells to generate hybridomas producing mAbs (see Example 1).

Hybridoma supernatants were screened by flow cytometry against at least two cell lines, one overexpressing hCCR8 and a corresponding control cell line not overexpressing CCR8, to identify mAbs that specifically bound to hCCR8 (see Example 2). To characterize the epitopes to which hCCR8-specific Abs bound, hybridoma culture supernatants were screened by ELISA to measure binding to a BSA-conjugated peptide corresponding to the N-terminus of CCR8 (SEQ ID NO: 74). Several mAbs were identified which specifically bound to hCCR8 by flow cytometry but did not bind by ELISA to the BSA-conjugated CCR8 N-terminal peptide, suggesting binding to an epitope of hCCR8 different from the N-terminal epitope (Example 2).

Accordingly, this disclosure describes an isolated Ab, preferably a mAb, or an antigen-binding portion thereof, that binds specifically to hCCR8 expressed on the surface of a cell, wherein the Ab or antigen-binding portion thereof binds to an epitope that is not located in the N-terminal domain of hCCR8, i.e., an epitope that is different from the epitope in the N-terminal domain of hCCR8 bound by the anti-CCR8 mAbs disclosed in WO 2021/194942. The amino acid sequence of hCCR8 is set forth as SEQ ID NO: 1. In certain embodiments, the N-terminal epitope comprises at least one amino acid within a peptide having the sequence Y₁₅Y₁₆Y₁₇P₁₈D₁₉I₂₀F₂₁ (SEQ ID NO: 2). In certain other embodiments, the N-terminal epitope comprises 2, 3, 4, 5, 6, or all 7 of the amino acids within the peptide having the sequence of SEQ ID NO: 2. In certain preferred embodiments, the N-terminal epitope comprises all 7 of the amino acids within the peptide having the sequence of SEQ ID NO: 2. In certain other embodiments, the N-terminal epitope comprises at least one amino acid within a peptide having the sequence V₁₂T₁₃D₁₄Y₁₅Y₁₆Y₁₇P₁₈D₁₉I₂₀F₂₁S₂₂ (SEQ ID NO: 73). In certain other embodiments, the N-terminal epitope comprises 2, 3, 4, 5, 6, 7, 8, 9, 10 or all 11 of the amino acids within the peptide having the sequence of SEQ ID NO: 73. In certain preferred embodiments, the N-terminal epitope comprises all 11 of the amino acids within the peptide having the of SEQ ID NO: 73. In further preferred embodiments, the amino acids Y₁₅ and/or Y₁₇ are sulfated.

Abs that Bind to an N-Terminal Epitope do not Interfere with the Binding of the mAbs of the Invention that Bind to Non-N-Terminal Epitopes of hCCR8

To determine whether the binding of mAb 4A19, an Ab that binds to an N-terminal domain epitope of hCCR8 (see WO 2021/194942), blocks the binding of mAb 21C17 of the present invention to CCR8, tissue from a dissociated gastric tumor was pre-incubated with unlabeled mAb 4A19 or unlabeled mAb 21C17, and then stained with immune marker Abs against CD3, CD8, CD4, FOXP3, and two anti-hCCR8 Abs, Clone L263G8 (BioLegend) or mAb 21C17. Analysis by flow cytometry (Example 3) showed that binding of mAb 4A19 to CCR8 blocks the subsequent binding of L263G8, but does not block the binding of mAb 21C17, to CCR8 on Tregs. Conversely, binding of 21C17 to CCR8 blocks the subsequent binding of 21C17 itself, but not the binding of L263G8 to Tregs. These results indicate that mAb 21C17 mAb binds to an epitope on CCR8 that is distinct from the N-terminal epitope to which mAb 4A19 binds, and the binding of 4A19 does not interfere with the binding of 21C17 to CCR8-expressing Tregs.

Accordingly, the present disclosure provides an isolated Ab, preferably a mAb, or an antigen-binding portion thereof of the invention, whose binding to hCCR8 is not affected by the presence of an Ab bound to an N-terminal epitope of hCCR8. In certain embodiments, the mAbs of the invention disclosed herein do not compete for binding to hCCR8 with any of mAbs mAb 4A19, 18Y12, 10R3, 8D55, 14S15, and 15S15h, described in WO 2021/194942. In certain other embodiments, binding of the mAbs of the invention to hCCR8 is not affected by the presence of an Ab bound to an epitope comprising 1, 2, 3, 4, 5, 6, 7, 8, 9, 10 or all 11 of the amino acids within a peptide domain of hCCR8 having the amino acid sequence V₁₂T₁₃D₁₄Y₁₅Y₁₆Y₁₇P₁₈D₁₉I₂₀F₂₁S₂₂ (SEQ ID NO: 73). In certain preferred embodiments, binding of mAb 21C17 to hCCR8 is not affected by the presence of an Ab bound to an epitope comprising, or consisting of, all 11 of the amino acids within a peptide domain of hCCR8 having the amino acid sequence V₁₂T₁₃D₁₄Y₁₅Y₁₆Y₁₇P₁₈D₁₉I₂₀F₂₁S₂₂ (SEQ ID NO: 73). In other preferred embodiments, binding of mAb 21C17 to hCCR8 is not affected by the presence of mAb4A19 (WO 2021/194942) bound to hCCR8.

In certain aspects of this invention, the Ab bound to the N-terminal epitope comprises:
(a) a heavy chain variable region CDR1 comprising consecutively linked amino acids having the sequence set forth as SEQ ID NO: 53; a heavy chain variable region CDR2 comprising consecutively linked amino acids having the sequence set forth as SEQ ID NO: 54; a heavy chain variable region CDR3 comprising consecutively linked amino acids having the sequence set forth as SEQ ID NO: 55; a light chain variable region CDR1 comprising consecutively linked amino acids having the sequence set forth as SEQ ID NO: 56; a light chain variable region CDR2 comprising consecutively linked amino acids having the sequence set forth as SEQ ID NO: 57; and a light chain variable region CDR3 comprising consecutively linked amino acids having the sequence set forth as SEQ ID NO: 58;
(b) a V_{H} comprising consecutively linked amino acids having the sequence set forth as SEQ ID NO: 9 and a V_{L} comprising consecutively linked amino acids having the sequence set forth as SEQ ID NO: 16; or
(c) a heavy chain comprising consecutively linked amino acids having the sequence set forth as SEQ ID NO: 65 and a light chain comprising consecutively linked amino acids having the sequence set forth as SEQ ID NO: 72.

In certain embodiments, the Ab bound to the N-terminal epitope, which does not interfere with the binding of an Ab of the invention, is mAb 4A19, 18Y12, 10R3, 8D55, 14S15, or 14S15h (see WO 2021/194942), or L263G8 (BioLegend). Preferably, the Ab bound to the N-terminal epitope, which does not interfere with the binding of an Ab of the invention, is mAb 4A19.

MAbs that Bind with High Affinity to Non-N-Terminal Epitopes on hCCR8

Certain of the anti-CCR8 mAbs of this invention bind specifically to hCCR8 with high affinity. Binding affinities for an Ab binding to a target such as hCCR8 can be determined by measuring the dissociation constant (K_{D}) or the half maximal effective concentration (EC₅₀) for binding to CCR8-expressing cell lines. The term “K_{D},” as used herein, is intended to refer to the dissociation constant for a particular Ab-antigen interaction, which is obtained from the ratio of k_{off} to kₒₙ (i.e., k_{off}/kₒₙ) and is expressed as a molar concentration (e.g., nM). The EC₅₀, also expressed as a molar concentration (e.g., nM), is the concentration of the Ab that achieves half of the maximal binding.

The binding specificity of Abs of the invention in binding to CCR8 was measured by fluorescent activated cell sorting (FACS) (see Example 4). The 6 mAbs tested were shown to bind to hCCR8-expressing Raji cells but not to the parental Raji cells that do not express CCR8 (Example 4). The FACS analysis identified 4 mAbs that bound to Raji-hCCR8 cells with an EC₅₀ below 1 nM (see Table 1).

Accordingly, in certain embodiments, the mAb or antigen-binding portion thereof of the invention specifically binds to hCCR8-expressing Raji cells with an EC₅₀ of:
(a) about 50 nM or lower;
(b) about 3 nM or lower to about 0.5 nM or lower;
(c) about 0.5 nM or lower;
(d) about 0.1 nM or lower;
(e) about 0.01 nM or lower;
(f) about 0.005 nM or lower;
(g) about 0.1 nM;
(h) between about 0.005 nM and about 50 nM;
(i) between about 0.02 nM and about 3 nM; or
(j) between about 0.08 nM and about 2 nM.
   In certain preferred embodiments, the EC₅₀ is measured by the binding assay described in Example 4.

When the binding affinity of an Ab to an antigen is expressed by a K_{D} or EC₅₀ having a specified value “or lower”, it does not mean that there is no lower limit to the K_{D} or EC₅₀ value, or that this value is infinitely low. In practice, the K_{D} or EC₅₀ value of an Ab that binds with very high affinity does not fall below the picomolar range, i.e., about 0.001 nM. Thus, a person skilled in the art would understand that an EC₅₀ of, for example, about 0.5 nM or lower, means that an Ab binds to an antigen with an EC₅₀ of about 0.5 nM, or an EC₅₀ lower than about 0.5 nM, but not lower than about 0.001 nM.

In certain other embodiments, the mAb or antigen-binding portion thereof of the invention binds to a cell surface-expressed hCCR8 polypeptide in a formalin-fixed, paraffin-embedded (FFPE) tissue sample.

Competitive Binding Between mAbs that do not Bind to N-Terminus of CCR8 vs. N-Terminus-Binding mAbs

Competition for binding to CCR8 on the surface of activated Tregs between an anti-CCR8 mAb that binds to the N-terminus of CCR8 (4A19-mIgG2a) and mAbs that do not bind to the N-terminus (23A14-hIgG1, 21C17-mIgG2a, and 22B13-mIgG2a) was assayed by FACS (see Example 4). Binding of mAb 4A19-mIgG2a to CCR8 did not interfere with the subsequent binding of any of 23A14-hIgG1, 21C17-mIgG2a or 22B13-mIgG2a, and both types of mAbs tested at saturating quantities were able to detect equal populations of CCR8⁺ Tregs.

Thus, the mAbs of the invention bind to one or more epitopes of hCCR8 that are distinct from the epitope(s) located in the N-terminal domain of hCCR8 to which therapeutic anti-CCR8 mAbs, such as mAb 4A19, described in WO 2021/194942 bind, and the presence of an Ab bound to an N-terminal epitope does not interfere with the binding of an Ab to the other epitope. The mAbs of the invention, that bind to a hCCR8 epitope outside of the N-terminal region, include 25T40, 21C17, 28P3, 22B13, 33H18 and 23A14.

Anti-CCR8 mAbs that Cross-Compete with a Reference Ab for Binding to CCR8

Also encompassed within the scope of the disclosed invention is an isolated Ab, preferably a mAb, or an antigen-binding portion thereof, which specifically binds to hCCR8 expressed on the surface of a cell, and cross-competes with a reference Ab or a reference antigen-binding portion thereof for binding to hCCR8. The ability of a pair of Abs to “cross-compete” for binding to an antigen, e.g., CCR8, indicates that a first Ab binds to substantially the same epitope region of the antigen as, and sterically hinders the binding of, a second Ab to that particular epitope region and, conversely, the second Ab binds to substantially the same epitope region of the antigen as, and sterically hinders the binding of, the first Ab to that epitope region. Thus, the ability of a test Ab to competitively inhibit the binding of, for example, mAb 25T40 or 21C17 to hCCR8, demonstrates that the test Ab binds to substantially the same epitope region of hCCR8 as does mAb 25T40 or 21C17.

A first Ab is considered to bind to “substantially the same epitope region” as does a second Ab if the first Ab reduces the binding of the second Ab to an antigen by at least about 40%. Preferably, the first Ab reduces the binding of the second Ab to the antigen by more than about 50% (e.g., at least about 60% or at least about 70%). In more preferred embodiments, the first Ab reduces the binding of the second Ab to the antigen by more than about 70% (e.g., at least about 80%, at least about 90%, or about 100%). The order of the first and second Abs can be reversed, i.e., the “second” Ab can be first bound to the surface and the “first” is thereafter brought into contact with the surface in the presence of the “second” Ab. The Abs are considered to “cross-compete” if a competitive reduction in binding to the antigen is observed irrespective of the order in which the Abs are added to the immobilized antigen.

An “epitope region” refers to an epitope and the spatial region around the epitope. Notably, Abs that bind to substantially the same “epitope region” of an antigen may not necessarily bind to the identical epitope (though Abs that bind to the same epitope can be identified by first screening for Abs that bind to the same epitope region, followed by epitope mapping of these Abs using techniques well known in the art including array-based oligo-peptide scanning, site-directed scanning mutagenesis mapping (e.g., alanine-scanning epitope mapping), high-throughput shotgun mutagenesis epitope mapping, hydrogen-deuterium exchange (HDX), and X-ray crystallography). For example, two cross-competing mAbs that bind to substantially the same epitope region of an antigen may bind to adjacent or overlapping, but non-identical, epitopes and sterically hinder each other's binding to their cognate epitopes. Alternatively, the binding of a mAb to one epitope may induce a conformational change in the antigen that reduces the binding of another mAb to a different epitope in substantially the same epitope region. Nevertheless, cross-competing Abs are generally expected to have very similar functional properties by virtue of their binding to substantially the same epitope region of an antigen such as a CCR8 receptor. The higher the degree of cross-competition, the more similar the functional properties are expected to be. For example, two cross-competing Abs are expected to have essentially the same functional properties if they each inhibit binding of the other to an epitope by at least about 80%, more so if they each inhibit binding of the other to an epitope by at least about 90%, and even more so if they each inhibit binding of the other to an epitope by about 100%. This similarity in function is expected to be even closer if the cross-competing Abs exhibit similar affinities for binding to the epitope as measured by the K_{D} or EC₅₀.

Cross-competing anti-antigen Abs can be readily identified based on their ability to detectably compete in standard antigen binding assays, including BIACORE® analysis, ELISA assays or flow cytometry, using either recombinant antigen molecules or cell-surface expressed antigen molecules. By way of example, a simple competition assay to identify whether a test Ab competes with mAb 21C17 for binding to hCCR8 may involve: (1) measuring the binding of 21C17, applied at saturating concentration, to a BIACORE® chip (or other suitable medium for SPR analysis) onto which hCCR8 is immobilized, and (2) measuring the binding of 21C17 to a hCCR8-coated BIACORE® chip (or other medium suitable) to which the test Ab has been previously bound. The binding of 21C17 to the hCCR8-1-coated surface in the presence and absence of the test Ab is compared. A significant (e.g., more than about 40%) reduction in binding of 21C17 in the presence of the test Ab indicates that both Abs recognize substantially the same epitope region such that they compete for binding to the hCCR8 target. The percentage by which the binding of a first Ab to an antigen is inhibited by a second Ab can be calculated as: [1−(detected binding of first Ab in presence of second Ab)/(detected binding of first Ab in absence of second Ab)]×100. To determine whether the Abs cross-compete, the competitive binding assay is repeated except that the binding of the test Ab to the hCCR8-coated chip in the presence of pre-bound mAb 21C17 is measured.

Any of the anti-CCR8 Abs disclosed herein that bind to an epitope of hCCR8 outside the N-terminal domain may serve as a reference Ab in cross-competition assays. Thus, for example, certain aspects of the presently disclosed invention relates to an isolated Ab, preferably a mAb, or an antigen-binding portions thereof, which cross-competes for binding to hCCR8 with a reference Ab, wherein the reference Ab comprises:
(a) a V_{H} comprising consecutively linked amino acids having the sequence set forth as SEQ ID NO: 3 and a V_{L} comprising consecutively linked amino acids having the sequence set forth as SEQ ID NO: 10;
(b) a V_{H} comprising consecutively linked amino acids having the sequence set forth as SEQ ID NO: 4 and a V_{L} comprising consecutively linked amino acids having the sequence set forth as SEQ ID NO: 11;
(c) a V_{H} comprising consecutively linked amino acids having the sequence set forth as SEQ ID NO: 5 and a V_{L} comprising consecutively linked amino acids having the sequence set forth as SEQ ID NO: 12;
(d) a V_{H} comprising consecutively linked amino acids having the sequence set forth as SEQ ID NO: 6 and a V_{L} comprising consecutively linked amino acids having the sequence set forth as SEQ ID NO: 13;
(e) a V_{H} comprising consecutively linked amino acids having the sequence set forth as SEQ ID NO: 7 and a V_{L} comprising consecutively linked amino acids having the sequence set forth as SEQ ID NO: 14; or
(f) a V_{H} comprising consecutively linked amino acids having the sequence set forth as SEQ ID NO: 8 and a V_{L} comprising consecutively linked amino acids having the sequence set forth as SEQ ID NO: 15.
   Anti-CCR8 mAbs that Bind to the Same Non-N-Terminal Binding CCR8 Epitope as does a Reference Ab

Certain other aspects of the invention relates to an isolated Ab, preferably a mAb, or an antigen-binding portions thereof, which binds to the same epitope as does a reference Ab, wherein the reference Ab comprises:
(a) a V_{H} comprising consecutively linked amino acids having the sequence set forth as SEQ ID NO: 3 and a V_{L} comprising consecutively linked amino acids having the sequence set forth as SEQ ID NO: 10;
(b) a V_{H} comprising consecutively linked amino acids having the sequence set forth as SEQ ID NO: 4 and a V_{L} comprising consecutively linked amino acids having the sequence set forth as SEQ ID NO: 11;
(c) a V_{H} comprising consecutively linked amino acids having the sequence set forth as SEQ ID NO: 5 and a V_{L} comprising consecutively linked amino acids having the sequence set forth as SEQ ID NO: 12;
(d) a V_{H} comprising consecutively linked amino acids having the sequence set forth as SEQ ID NO: 6 and a V_{L} comprising consecutively linked amino acids having the sequence set forth as SEQ ID NO: 13;
(e) a V_{H} comprising consecutively linked amino acids having the sequence set forth as SEQ ID NO: 7 and a V_{L} comprising consecutively linked amino acids having the sequence set forth as SEQ ID NO: 14; or
(f) a V_{H} comprising consecutively linked amino acids having the sequence set forth as SEQ ID NO: 8 and a V_{L} comprising consecutively linked amino acids having the sequence set forth as SEQ ID NO: 15.
   Structurally Defined mAbs of the Invention

Certain other aspects of the invention relate to an isolated Ab, preferably a mAb, or an antigen-binding portions thereof, which specifically binds to hCCR8 expressed on the surface of a cell, and comprises the CDR1, CDR2 and CDR3 domains in each of:
(a) a V_{H} comprising consecutively linked amino acids having the sequence set forth as SEQ ID NO: 3 and a V_{L} comprising consecutively linked amino acids having the sequence set forth as SEQ ID NO: 10;
(b) a V_{H} comprising consecutively linked amino acids having the sequence set forth as SEQ ID NO: 4 and a V_{L} comprising consecutively linked amino acids having the sequence set forth as SEQ ID NO: 11;
(c) a V_{H} comprising consecutively linked amino acids having the sequence set forth as SEQ ID NO: 5 and a V_{L} comprising consecutively linked amino acids having the sequence set forth as SEQ ID NO: 12;
(d) a V_{H} comprising consecutively linked amino acids having the sequence set forth as SEQ ID NO: 6 and a V_{L} comprising consecutively linked amino acids having the sequence set forth as SEQ ID NO: 13;
(e) a V_{H} comprising consecutively linked amino acids having the sequence set forth as SEQ ID NO: 7 and a V_{L} comprising consecutively linked amino acids having the sequence set forth as SEQ ID NO: 14; or
(f) a V_{H} comprising consecutively linked amino acids having the sequence set forth as SEQ ID NO: 8 and a V_{L} comprising consecutively linked amino acids having the sequence set forth as SEQ ID NO: 15.

Different methods have been developed to delineate the CDR domains within an Ab. The approach of Kabat and co-workers (Wu and Kabat, 1970; Kabat et al., 1983), was based on the assumption that CDRs include the most variable positions in Abs and therefore could be identified by aligning the fairly limited number of Ab sequences then available. Based on this alignment, Kabat et al. introduced a numbering scheme for the residues in the hypervariable regions and determined which positions mark the beginning and the end of each CDR (http://bioinf.org.uk/abs/simkab.html).

In addition to the widely used Kabat definition, others including the Chothia (Chothia et al., 1987; 1989; Al-Lazikani et al., 1997; http://bioinforg.uk/abs/chothia.html), AbNum (Abhinandan and Martin, 2008; see AbNum; available at http://www.bioinf.org.uk/abs/abnum/), AbM (http://www.bioinforg.uk/abs; Martin et al., 1989), contact (http://bioinforg.uk/abs/; MacCallum et al., 1996) and IMGT (Lefranc et al., 2003; http://www.imgt.org) definitions that seek to address deficiencies of the Kabat definitions, have been employed. The Kabat definition is still the most commonly used method to predict CDR domains, notwithstanding it was developed when no structural information on Abs was available.

Where not explicitly stated, and unless the context indicates otherwise, CDRs disclosed herein have been identified using the Kabat definitions. The amino acid sequences for the 6 CDR domains as defined using the Kabat method, as well as the amino acid sequences for the V_{H}, V_{L}, heavy chain and light chain for mAbs 25T40, 21C17, 28P3, 22B13, 33H18, and 23A14 are shown in Table 3.

In certain other embodiments, the Ab, preferably a mAb, or antigen-binding portion thereof of the invention comprises the following CDR domains as defined by the Kabat method:
(a) a heavy chain variable region CDR1 comprising consecutively linked amino acids having the sequence set forth as SEQ ID NO: 17; a heavy chain variable region CDR2 comprising consecutively linked amino acids having the sequence set forth as SEQ ID NO: 18; a heavy chain variable region CDR3 comprising consecutively linked amino acids having the sequence set forth as SEQ ID NO: 19; a light chain variable region CDR1 comprising consecutively linked amino acids having the sequence set forth as SEQ ID NO: 20; a light chain variable region CDR2 comprising consecutively linked amino acids having the sequence set forth as SEQ ID NO: 21; and a light chain variable region CDR3 comprising consecutively linked amino acids having the sequence set forth as SEQ ID NO: 22;
(b) a heavy chain variable region CDR1 comprising consecutively linked amino acids having the sequence set forth as SEQ ID NO: 23; a heavy chain variable region CDR2 comprising consecutively linked amino acids having the sequence set forth as SEQ ID NO: 24; a heavy chain variable region CDR3 comprising consecutively linked amino acids having the sequence set forth as SEQ ID NO: 25; a light chain variable region CDR1 comprising consecutively linked amino acids having the sequence set forth as SEQ ID NO: 26; a light chain variable region CDR2 comprising consecutively linked amino acids having the sequence set forth as SEQ ID NO: 27; and a light chain variable region CDR3 comprising consecutively linked amino acids having the sequence set forth as SEQ ID NO: 28;
(c) a heavy chain variable region CDR1 comprising consecutively linked amino acids having the sequence set forth as SEQ ID NO: 29; a heavy chain variable region CDR2 comprising consecutively linked amino acids having the sequence set forth as SEQ ID NO: 30; a heavy chain variable region CDR3 comprising consecutively linked amino acids having the sequence set forth as SEQ ID NO: 31; a light chain variable region CDR1 comprising consecutively linked amino acids having the sequence set forth as SEQ ID NO: 32; a light chain variable region CDR2 comprising consecutively linked amino acids having the sequence set forth as SEQ ID NO: 33; and a light chain variable region CDR3 comprising consecutively linked amino acids having the sequence set forth as SEQ ID NO: 34;
(d) a heavy chain variable region CDR1 comprising consecutively linked amino acids having the sequence set forth as SEQ ID NO: 35; a heavy chain variable region CDR2 comprising consecutively linked amino acids having the sequence set forth as SEQ ID NO: 36; a heavy chain variable region CDR3 comprising consecutively linked amino acids having the sequence set forth as SEQ ID NO: 37; a light chain variable region CDR1 comprising consecutively linked amino acids having the sequence set forth as SEQ ID NO: 38; a light chain variable region CDR2 comprising consecutively linked amino acids having the sequence set forth as SEQ ID NO: 39; and a light chain variable region CDR3 comprising consecutively linked amino acids having the sequence set forth as SEQ ID NO: 40;
(e) a heavy chain variable region CDR1 comprising consecutively linked amino acids having the sequence set forth as SEQ ID NO: 41; a heavy chain variable region CDR2 comprising consecutively linked amino acids having the sequence set forth as SEQ ID NO: 42; a heavy chain variable region CDR3 comprising consecutively linked amino acids having the sequence set forth as SEQ ID NO: 43; a light chain variable region CDR1 comprising consecutively linked amino acids having the sequence set forth as SEQ ID NO: 44; a light chain variable region CDR2 comprising consecutively linked amino acids having the sequence set forth as SEQ ID NO: 45; and a light chain variable region CDR3 comprising consecutively linked amino acids having the sequence set forth as SEQ ID NO: 46; or
(f) a heavy chain variable region CDR1 comprising consecutively linked amino acids having the sequence set forth as SEQ ID NO: 47; a heavy chain variable region CDR2 comprising consecutively linked amino acids having the sequence set forth as SEQ ID NO: 48; a heavy chain variable region CDR3 comprising consecutively linked amino acids having the sequence set forth as SEQ ID NO: 49; a light chain variable region CDR1 comprising consecutively linked amino acids having the sequence set forth as SEQ ID NO: 50; a light chain variable region CDR2 comprising consecutively linked amino acids having the sequence set forth as SEQ ID NO: 51; and a light chain variable region CDR3 comprising consecutively linked amino acids having the sequence set forth as SEQ ID NO: 22.

In further embodiments, the Ab, preferably a mAb, or antigen-binding portion thereof comprises:
(a) a V_{H} comprising consecutively linked amino acids having the sequence set forth as SEQ ID NO: 3 and a V_{L} comprising consecutively linked amino acids having the sequence set forth as SEQ ID NO: 10;
(b) a V_{H} comprising consecutively linked amino acids having the sequence set forth as SEQ ID NO: 4 and a V_{L} comprising consecutively linked amino acids having the sequence set forth as SEQ ID NO: 11;
(c) a V_{H} comprising consecutively linked amino acids having the sequence set forth as SEQ ID NO: 5 and a V_{L} comprising consecutively linked amino acids having the sequence set forth as SEQ ID NO: 12;
(d) a V_{H} comprising consecutively linked amino acids having the sequence set forth as SEQ ID NO: 6 and a V_{L} comprising consecutively linked amino acids having the sequence set forth as SEQ ID NO: 13;
(e) a V_{H} comprising consecutively linked amino acids having the sequence set forth as SEQ ID NO: 7 and a V_{L} comprising consecutively linked amino acids having the sequence set forth as SEQ ID NO: 14; or
(f) a V_{H} comprising consecutively linked amino acids having the sequence set forth as SEQ ID NO: 8 and a V_{L} comprising consecutively linked amino acids having the sequence set forth as SEQ ID NO: 15.

Anti-CCR8 Abs comprising V_{H} and V_{L} regions having amino acid sequences that are highly similar or homologous to the amino acid sequences of any of the above anti-CCR8 Abs and which retain the functional properties of these Abs are also suitable for use in the present methods. For example, suitable Abs include mAbs comprising a V_{H} and/or V_{L} region each comprising consecutively linked amino acids having a sequence that is at least about 80% identical to the amino acid sequence set forth in SEQ ID Nos. 4 and/or 11, respectively. In further embodiments, for example, the V_{H} and/or V_{L} amino acid sequences exhibits at least about 85%, at least about 90%, at least about 95%, or at least about 99% identity to the sequences set forth in SEQ ID Nos. 4 and/or 11, respectively. As used herein, the percent sequence identity between two amino acid sequences is a function of the number of identical positions shared by the sequences relative to the length of the sequences compared (i.e., % identity=number of identical positions/total number of positions being compared×100), taking into account the number of any gaps, and the length of each such gap, introduced to maximize the degree of sequence identity between the two sequences. The comparison of sequences and determination of percent identity between two sequences can be accomplished using mathematical algorithms that are well known to those of ordinary skill in the art.

In certain embodiments of the Abs, preferably mAbs, that comprise V_{H} and/or V_{L} amino acid sequences exhibiting high sequence identity, e.g., at least about 85%, at least about 90%, at least about 95%, or at least about 99% identity to an Ab that is structurally defined herein, there are no more than 3 amino acid modifications in each of the V_{H} and V_{L} CDR domains compared to the CDR sequences of the defined Ab. In certain preferred embodiments, there are no more than 2 amino acid modifications in each of the V_{H} and V_{L} CDR domains. In certain more preferred embodiments, there is no more than 1 amino acid modification in each of the V_{H} and V_{L} CDR domains. In certain even more preferred embodiments, there is no amino acid modification in each of the V_{H} and V_{L} CDR domains.

In preferred embodiments of Abs comprising one or more amino acid modifications in the CDRs, these modifications are “conservative” amino acid modifications. As used herein, a “conservative” amino acid modification refers to an amino acid modification that does not significantly affect or alter the binding characteristics of the Ab containing the amino acid sequence. Such conservative modifications include amino acid substitutions, insertions and deletions. Conservative amino acid substitutions are ones in which the amino acid residue is replaced with an amino acid residue having a similar side chain. Families of amino acid residues having similar side chains have been defined in the art and include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, in a conservative amino acid substitution, one or more amino acid residues within the CDR regions of an Ab is replaced by another amino acid residue from the same side-chain family and the altered Ab is tested, using assays well known in the art, to verify whether Ab function, e.g., binding specificity and affinity, is substantially the same as the unmodified Ab.

In certain other embodiments, the Ab, preferably a mAb, or antigen-binding portion thereof comprises:
(a) a heavy chain comprising consecutively linked amino acids having the sequence set forth as SEQ ID NO: 59 and a light chain comprising consecutively linked amino acids having the sequence set forth as SEQ ID NO: 66;
(b) a heavy chain comprising consecutively linked amino acids having the sequence set forth as SEQ ID NO: 60 and a light chain comprising consecutively linked amino acids having the sequence set forth as SEQ ID NO: 67;
(c) a heavy chain comprising consecutively linked amino acids having the sequence set forth as SEQ ID NO: 61 and a light chain comprising consecutively linked amino acids having the sequence set forth as SEQ ID NO: 68;
(d) a heavy chain comprising consecutively linked amino acids having the sequence set forth as SEQ ID NO: 62 and a light chain comprising consecutively linked amino acids having the sequence set forth as SEQ ID NO: 69;
(e) a heavy chain comprising consecutively linked amino acids having the sequence set forth as SEQ ID NO: 63 and a light chain comprising consecutively linked amino acids having the sequence set forth as SEQ ID NO: 70; or
(f) a heavy chain comprising consecutively linked amino acids having the sequence set forth as SEQ ID NO: 64 and a light chain comprising consecutively linked amino acids having the sequence set forth as SEQ ID NO: 71.

In certain preferred embodiments, the mAb or antigen-binding portion thereof is the Ab designated herein as 25T40, 21C17, 28P3, 22B13, 33H18, or 23A14, or an antigen-binding portion thereof.

In certain embodiments, the isolated anti-CCR8 Ab, preferably a mAb, or antigen-binding portion thereof of this invention is a human Ab or fragment thereof. In other embodiments, it is a humanized Ab or fragment thereof. In further embodiments, it is a chimeric Ab or fragment thereof. In other embodiments, the isolated anti-CCR8 Ab or antigen-binding portion thereof is a mouse Ab or fragment thereof. For use in human subjects, the Abs are preferably chimeric Abs or, more preferably, humanized or human Abs. Such chimeric, humanized, human or mouse mAbs can be prepared and isolated by methods well known in the art.

In certain other embodiments, the Ab, preferably a mAb, or antigen-binding portion thereof comprises a heavy chain constant region which is of a human IgG1, IgG2, IgG3 or IgG4 isotype. In further embodiments, the Ab or antigen-binding portion thereof comprises a heavy chain constant region which is of a human IgG2 or IgG4 isotype.

MAbs Labeled with Detectable Tags

MAbs can be labeled with various substances or tags, including small molecules, enzymes, radioisotopes and fluorescent dyes, to aid in detection. The type of label used is dependent on the downstream applications. For example, a reporter enzyme or biotin is typically used for enzyme-linked immunoassay (ELISA) and immunohistochemistry (IHC). These labels, as well as fluorescent labels are used for Western blots, while fluorescent labels are used for flow cytometry and immunofluorescence (IF) staining. Radiolabeled mAbs are used for positron emission tomography (PET) (Aluicio-Sarduy et al., 2018), and rare earth metal isotopes are used for mass cytometry.

This disclosure provides a labeled Ab, preferably a mAb, or an antigen-binding thereof, comprising any one of the aforementioned Abs of the invention, or an antigen-binding portion thereof, and a detectable label. In certain embodiments, the detectable label is a fluorophore, an enzyme, a micropolymer, a radioactive isotope, or a metal. In certain other embodiments, the detectable label is biotin. In further embodiments, the fluorophore is a BRILLIANT VIOLET™ dye (e.g., BV-421), an AmCyan dye, an ALEXA FLUOR® dye, a CY® dye, a CF® dye, fluorescein isothiocyanate (FITC), tetramethylrhodamine (TRITC) phycoerythrin (PE), allophycocyanin (APC), or peridinin-chlorophyll protein (PerCP). In certain other embodiments, the enzyme is alkaline phosphatase, horseradish peroxidase, glucose oxidase or β-galactosidase. In further embodiments, the chromophore is a porphyrin, pyropheophorbide-α, benzoporphyrin monoacid ring A (BPDMA), or chlorin e6.

The radiolabeling of mAbs with positron emitters for PET imaging (immunoPET) may provide valuable information about the in vivo biodistribution of these molecules and their related therapeutics (Aluicio-Sarduy et al., 2018). For each PET application, the selection of the optimal radioisotope is crucial. It starts by matching the half-life of the radionuclide with the pharmacokinetic profile of the mAb in vivo. This ensures that the time course of the radioactivity matches that of the mAb. Typically, due to their prolonged half-lives in the bloodstream, the accumulation of mAbs in tumors tends to peak days after injection, which makes necessary the use of long half-life isotopes (e.g., ⁸⁹Zr, ⁶⁴Cu, and ⁸⁶Y) instead of more traditional choices such as ¹¹C, ¹⁸F, or ⁶⁸Ga. In instances where the conventional isotopes do not suit the desired application, other radionuclides have been investigated which offer more appropriate chemical or decay properties. Notable examples of such alternative radionuclides include ⁵²Mn, ⁵⁵Co, ¹⁵²Tb, ⁹⁰Nb ⁶⁶Ga, ⁷²As, and ⁶⁹Ge (Aluicio-Sarduy et al., 2018). Currently, ⁸⁹Zr is utilized in clinical trials much more extensively than any other PET-radiometal. Its 78-h half-life matches the typical pharmacokinetic time scales of mAbs and makes ⁸⁹Zr well suited for centralized production and national and international transport. ⁶⁴Cu represents another convenient alternative for Ab and protein labeling as it has an intermediate half-life of 12.7 h and negligible contaminating gamma ray emissions. ⁸⁶Y, with a half-life of 14.7 h, is another isotope with promising characteristics for immunoPET but it is a significant gamma ray emitter which limits the quantities that can be injected to a subject.

In certain preferred embodiments of the mAb labeled for PET, the radioactive isotope is ⁸⁹Zr, ⁶⁴Cu or ⁸⁶Y. In certain other embodiments, the radioactive isotope is ¹¹C, ¹⁸F, ⁶⁸Ga ⁵²Mn, ⁵⁵Co, ¹⁵²Tb, ⁹⁰Nb, ⁶⁶Ga, ⁷²As, ⁶⁹Ge, or I¹²⁵.

Mass cytometry, also called cytometry by time-of-flight (CyTOF), is a next-generation flow cytometry platform which utilizes elemental mass spectrometry to detect rare metal isotopes conjugated to Abs bound intracellularly or extracellularly to antigens of interest on single cells (Spitzer and Nolan, 2016). In this technique, cells are stained with Abs conjugated to metal isotope reporters with different mass. Then fixed, stained cells, divided into single cell droplets, are nebulized and analyzed by mass spectrometry. The number of cellular parameters that can be simultaneously monitored by conventional fluorescence flow cytometry assays is inherently limited by fluorophore emission spectra overlap, but mass cytometry accurately discriminates metal isotopes of different atomic masses without channel overlap. This allows more than 40 protein parameters to be simultaneously quantified within each single cell, much greater than the approximately 20 cellular features that can be simultaneously analyzed by fluorescence flow cytometry. Compared to flow cytometry, mass spectrometry yields analogous quantification of cell lineages in conjunction with markers of cell differentiation, function, activation, and exhaustion for use with fresh and viably frozen PBMC or tumor tissues (Gadalla et al., 2019). However, the throughput (˜1,000 cells/s) and sensitivity of mass cytometry are still about 10-fold lower than conventional FACS and, as the cells are totally “evaporated” during the assay, cells analyzed by mass cytometry cannot be retrieved for downstream analysis (Spitzer and Nolan, 2016).

In certain embodiments of the Ab labeled with a metal isotope, the metal is yttrium (Y), indium (In), the series of lanthanide elements (Ln, from La to Lu, except Pm), iodine (I), cadmium (Cd), tellurium (Te), silver (Ag), palladium (Pd), rhodium (Rh), iridium (Ir), platinum (Pt), ruthenium (Ru), osmium (Os) or bismuth (Bi).

### Anti-CCR8 Immunoconjugates

In another aspect, the present invention relates to any one of the isolated anti-hCCR8 Abs of the invention disclosed herein, or an antigen-binding portion thereof, linked to a cytolytic agent, such as a cytotoxin, a radioactive isotope, or a photosensitizer (PS). Such conjugates are referred to herein as “immunoconjugates”. Cytotoxins can be conjugated to Abs of the invention using linker technology available in the art. Methods for preparing radioimmunoconjugates are also established in the art.

Photodynamic therapy (PDT) is a non-invasive treatment that involves the accumulation of a PS in solid tumors followed by the localized delivery of light of the correct wavelength to cause activation of the PS, which, in the presence of oxygen, leads to the in situ generation of reactive oxygen species (ROS) that cause damage to cellular components and, ultimately, necrosis or apoptosis. PDT is a promising tool in oncology but is frequently limited by side-effects caused by inadequate targeting of the photosensitizer. This problem can often be circumvented by the conjugation of PS's to tumor-specific mAbs. The use of antigen-binding Ab fragments, e.g., Fab or scFv fragments, may be advantageous because while retaining the same binding specificity, they are more efficient at penetrating tumor masses due to their smaller size and are more effectively cleared from the circulation because of the lack of an Fc domain.

Porphyrins are ubiquitous in the fields of photodynamic therapy and photodiagnosis, and are one of the most prominent classes of photosensitizer in these areas of biomedical science (Sandland and Boyle, 2019). In certain embodiments of the photosensitizer-Ab conjugate, the photosensitizer is a tetrapyrrolic macrocycle. In further embodiments, the tetrapyrrolic macrocycle is a porphyrin, a chlorin, a bacteriochlorin, or a phthalocyanine.

### Anti-CCR8 Chimeric Antigen Receptors (CARs) and T Cell Receptors (TCRs)

In another aspect, the present invention relates to a chimeric antigen receptor (CAR) comprising any one of the isolated anti-hCCR8 Abs of the invention disclosed herein, or an antigen-binding portion thereof, that binds specifically to an epitope of hCCR8 outside the N-terminal domain of hCCR8. In certain embodiments, the CAR further comprises a transmembrane domain. In certain other embodiments, the CAR further comprises an intracellular signaling domain. In further embodiments, the CAR further comprises a hinge region and/or a spacer region.

In another aspect, the invention relates to a T cell receptor (TCR) comprising an antigen-binding region disclosed herein that binds specifically to an epitope of hCCR8 outside the N-terminal domain of hCCR8. In certain embodiments, the TCR further comprises a transmembrane domain. In certain other embodiments, the TCR further comprises an intracellular signaling domain.

### Bispecific Molecules

In another aspect, the present invention relates to a bispecific molecule comprising any one of the anti-hCCR8 mAbs of the invention disclosed herein, or an antigen-binding portion thereof, linked to a binding domain that has a different binding specificity than the anti-hCCR8 mAb or antigen-binding portion thereof. The binding domain may be a functional molecule, e.g., another Ab, an antigen-binding portion of an Ab, or a ligand for a receptor, such that the bispecific molecule generated binds to at least two different binding sites or target molecules.

### Pharmaceutical Compositions

Abs disclosed herein and used in the any of the diagnostic, patient selection and therapeutic methods described may be constituted in a composition, e.g., a pharmaceutical composition comprising any of the Abs of the invention and a pharmaceutically acceptable carrier. This disclosure also provides compositions e.g., pharmaceutical compositions, comprising any of the disclosed immunoconjugates, bispecific molecules, CARs, and TCRs, and a pharmaceutically acceptable carrier. As used herein, a “pharmaceutically acceptable carrier” includes any and all solvents, dispersion media, coatings, antibacterial and antifungal agents, isotonic and absorption delaying agents, and the like that are physiologically compatible. Preferably, the carrier for a composition containing an Ab is suitable for intravenous (IV), intramuscular, subcutaneous (SC), parenteral, spinal or epidermal administration (e.g., by injection or infusion). A pharmaceutical composition may include one or more pharmaceutically acceptable salts, anti-oxidants, aqueous and non-aqueous carriers, and/or adjuvants such as preservatives, wetting agents, emulsifying agents and dispersing agents.

An option for SC injection is based on Halozyme Therapeutics' ENHANZE® drug-delivery technology, involving a co-formulation of an Ab with recombinant human hyaluronidase enzyme (rHuPH20) that removes traditional limitations on the volume of biologics and drugs that can be delivered subcutaneously due to the extracellular matrix (U.S. Pat. No. 7,767,429). It may be possible to co-formulate two Abs used in combination therapy into a single composition for SC administration.

### Nucleic Acids Encoding Anti-hCCR8 MAbs and Use for Expressing Abs

Another aspect of the disclosed invention pertains to nucleic acids (nucleic acids of the invention) that encode any of the isolated anti-hCCR8 Abs of the invention. This disclosure provides an isolated nucleic acid encoding any of the anti-CCR8 mAbs of the invention described herein, or antigen-binding portions thereof, any bispecific Ab, chimeric antigen receptor (CAR), or T cell receptor (TCR), disclosed herein.

An “isolated” nucleic acid primarily refers to a nucleic acid composition of matter that is markedly different, i.e., has a distinctive chemical identity, nature and utility, from nucleic acids as they exist in nature. For example, an isolated DNA, unlike native DNA, is a free-standing portion of a native DNA and not an integral part of a larger structural complex, the chromosome, found in nature. Further, an isolated DNA, unlike native DNA, can be used as a PCR primer or a hybridization probe for, among other things, measuring gene expression and detecting biomarker genes or mutations for diagnosing disease or predicting the efficacy of a therapeutic. In a narrower sense, where the context indicates, the term “isolated” may also be used to describe a nucleic acid that is different from nucleic acids as they exist in nature in the sense that it is purified so as to be substantially free of other cellular components or other contaminants, e.g., other cellular nucleic acids or proteins, using standard techniques well known in the art.

Nucleic acids of the invention can be obtained using standard molecular biology techniques. For Abs expressed by hybridomas (e.g., hybridomas prepared from wild-type or transgenic mice carrying human Ig genes as described in Example 1), cDNAs encoding the light and heavy chains or variable regions of the Ab made by the hybridoma can be obtained by standard PCR amplification techniques. Once DNA fragments encoding V_{H} and V_{L} segments are obtained, these DNA fragments can be further manipulated using standard recombinant DNA techniques, for example, to convert the variable region DNAs to full-length Ab chain genes, to Fab fragment genes, or to a scFv gene. For Abs obtained from an Ig gene library (e.g., using phage display techniques), nucleic acids encoding the Ab can be recovered from the library.

A nucleic acid of the invention can be, for example, RNA, or DNA such as cDNA or genomic DNA. In preferred embodiments, the nucleic acid is a cDNA.

The present disclosure also provides an expression vector comprising an isolated nucleic that encodes an anti-CCR8 mAb or antigen-binding portion thereof disclosed herein, any bispecific Ab, chimeric antigen receptor (CAR) or T cell receptor (TCR) disclosed herein. The disclosure further provides a host cell comprising said expression vector, or any CAR or TCR disclosed herein. Eukaryotic cells, and most preferably mammalian host cells, are preferred as host cells for expressing Abs because such eukaryotic cells, and in particular mammalian cells, are more likely than prokaryotic cells to assemble and secrete a properly folded and immunologically active Ab. Preferred mammalian host cells for expressing the recombinant Abs of the invention include Chinese Hamster Ovary (CHO) cells (Kaufman and Sharp, 1982), NSO myeloma cells, COS cells, and SP2 cells. In certain embodiments, the host cell is an immune cell. In further embodiments, the host cell is a T or an NK cell.

The host cell may be used in a method for preparing an anti-CCR8 mAb or an antigen-binding portion thereof, a bispecific Ab, a CAR, or a TCR, which method comprises expressing the mAb or antigen-binding portion thereof, bispecific Ab, CAR, or TCR, in the host cell and isolating the mAb or antigen-binding portion thereof, bispecific Ab, CAR, or TCR, from the host cell. The host cell may be used ex vivo or in vivo. The DNAs encoding the Ab heavy and light chains can be inserted into separate expression vectors or, more typically, are both inserted into the same vector. The V_{H} and V_{L} segments of an Ab can be used to create full-length Abs of any isotype by inserting DNAs encoding these variable regions into expression vectors already encoding heavy chain and light chain constant regions of the desired isotype such that the V_{H} segment is operatively linked to the C_{H} segment(s) within the vector and the V_{κ} segment is operatively linked to the C_{L} segment within the vector.

### Anti-CCR8 Abs Suitable for Use in the Disclosed Therapeutic Methods

Disclosed herein are therapeutic methods that include a screening or diagnostic step involving the use of an anti-CCR8 Ab of the invention. An anti-CCR8 Ab suitable for use in these methods is an isolated Ab, preferably a mAb or antigen-binding portion thereof, that binds specifically to an epitope located outside the N-terminal domain of hCCR8 expressed on the surface of a cell. Such an Ab exhibits one or more properties that are important for the screening or diagnostic applications described herein. In particular, the isolated Ab or antigen-binding portion thereof:
(a) binds to an epitope that is outside the N-terminal domain of hCCR8, optionally to an epitope that is different from an epitope comprising a peptide having the sequence Y₁₅Y₁₆Y₁₇P₁₈D₁₉I₂₀F₂₁ (SEQ ID NO: 2);
(b) binds to an epitope that is outside the N-terminal domain of hCCR8, optionally to an epitope that is different from an epitope comprising a peptide having the sequence V₁₂T₁₃D₁₄Y₁₅Y₁₆Y₁₇P₁₈D₁₉I₂₀F₂₁S₂₂ (SEQ ID NO: 73); or
(c) binds to an epitope that is outside the N-terminal domain of hCCR8, optionally to an epitope that is different from an epitope consisting of a peptide having the sequence V₁₂T₁₃D₁₄Y₁₅Y₁₆Y₁₇P₁₈D₁₉I₂₀F₂₁S₂₂ (SEQ ID NO: 73).

In additional embodiments, the isolated Ab or antigen-binding portion thereof preferably exhibits at least one, at least two, or at least three of the following properties:
(d) specifically binds to hCCR8 expressed on the surface of a cell with an EC₅₀ of about 3 nM or lower, e.g., with an EC₅₀ of between about 0.01 and about 3 nM; preferably with an EC₅₀ of about 0.1 nM or lower, e.g., with an EC₅₀ of between about 0.001 and about 0.1 nM;
(e) binds to a cell surface-expressed hCCR8 polypeptide in a formalin-fixed, paraffin-embedded (FFPE) tissue sample;
(f) its binding to hCCR8 is not affected by the presence of an Ab or antigen-binding portion thereof, e.g., mAb 4A19 (WO 2021/194942), bound to an N-terminal epitope; and
(g) its binding to hCCR8 does not affect the binding of an Ab or antigen-binding portion thereof, e.g., mAb 4A19 (WO 2021/194942), to an N-terminal epitope.

In preferred embodiments, the isolated Ab or antigen-binding portion thereof exhibits at least 6 of the aforementioned properties (a) to (g), e.g., exhibits properties ((a) to (d), (f) and (g).

### Diagnostic, Theranostic, and Other Methods Employing Anti-CCR8 MAbs

PCT Publication No. WO 2021/194942 describes humanized and human mAbs that specifically bind with high affinity to hCCR8 expressed on the surface of CCR8-expressing tumor-infiltrating Tregs and mediate the depletion of these Tregs by mechanisms including ADCC. That application also describes methods of treating a cancer in a subject comprising administering to the subject a therapeutically effective amount of an anti-CCR8 mAb, which results in depletion of the immunosuppressive tumor-infiltrating Tregs and thereby enhancing the immune response in the subject to effectively treat the cancer. At least one of the therapeutic Treg-depleting mAbs, mAb 4A19, binds to an epitope in the N-terminal domain of hCCR8 comprising residues 12-22 of hCCR8 (SEQ ID NO: 1) and including sulfated tyrosine-15 and tyrosine-17 residues.

The mAbs of the present invention bind specifically to hCCR8 but to a distinct epitope that is outside the N-terminal domain and, thus, differs from the N-terminal epitope bound by the therapeutic anti-CCR8 mAbs disclosed in WO 2021/194942. These mAbs of the invention, when bound to hCCR8, do not interfere with the binding of the therapeutic Abs that bind to the N-terminal domain; similarly, binding of the therapeutic N-terminal binding mAbs does not interfere with the binding of the mAbs of the invention. Thus, the present mAbs can be used in a variety of different methods that can be performed even in the presence of the bound therapeutic mAbs, including methods for measuring the frequency of CCR8⁺ cells, such as Tregs, in the blood or tumor environment in a subject; measuring a depletion in the number of CCR8⁺ cells, such as Tregs, in the blood or tumor environment in a subject; measuring receptor occupancy (RO), i.e., the proportion of hCCR8 receptors bound by an anti-CCR8 therapeutic antibody, in a subject being treated with the therapeutic antibody; predicting the effectiveness of a therapeutic anti-CCR8 Ab in treating a cancer in a subject; selecting a subject afflicted with a cancer as a suitable candidate for immunotherapy with a therapeutic Treg-depleting anti-CCR8 Ab; and therapeutic methods comprising determining the level of expression of CCR8 in a test tissue in or taken from the subject prior to administering a therapeutic Treg-depleting anti-CCR8 mAb.

A method for detecting and quantifying CCR8 expression on peripheral Tregs is described in Example 6. For detection and quantification of CCR8 in tumor tissue, single stain immunohistochemistry (IHC) was used (see Example 7).

As described in WO 2021/194942, CCR8⁺ cells in a tumor environment or in the peripheral blood are predominantly Tregs. It is advantageous that the anti-CCR8 mAb used to measure the frequency of CCR8⁺ cells in a tumor environment or in the peripheral blood does not compete for binding to CCR8 with a therapeutic anti-CCR8 mAb. In certain embodiments, the therapeutic anti-CCR8 mAb binds to an epitope in the N-terminal domain of hCCR8. In further embodiments, the therapeutic anti-CCR8 mAb is one of mAbs 4A19, 18Y12, 8D55, 10R3, 14S15 and 14S15h described in WO 2021/194942. In certain preferred embodiments, the therapeutic anti-CCR8 mAb is mAb 4A19 comprising the 6 CDRs (SEQ ID NOs. 53-58 herein), the heavy and/or light variable regions (SEQ ID NO: 9 and/or 16 herein), and/or the heavy and/or light chains (SEQ ID NO: 65 and/or 72 herein) of mAb 4A19.

In certain embodiments of such methods comprising use of a therapeutic anti-CCR8 mAb that binds to an epitope in the N-terminal domain of hCCR8, the non-competing mAb is an Ab that binds to an epitope outside of the N-terminal domain and does not compete for binding to hCCR8 with a mAb that binds to an epitope in the N-terminal domain. Examples include the Abs of the invention disclosed herein, i.e., one of mAbs designated herein as 25T40, 21C17, 28P3, 22B13, 33H18, or 23A14, or an antigen-binding portion thereof. In certain preferred embodiments, the non-competing mAb is mAb 21C17 or an antigen binding portion thereof comprising the 6 CDRs (SEQ ID NOs. 23-28), or the heavy and/or light variable regions (SEQ ID NO: 4 and/or 11), or the heavy and/or light chains (SEQ ID NO: 60 and/or 67) of mAb 21C17.

In certain other embodiments, the therapeutic anti-CCR8 mAb binds to an epitope outside the N-terminal domain of hCCR8. For example, in further embodiments, the therapeutic anti-CCR8 mAb is mAb VHH-01 disclosed in WO 2022/003156, or and of mAbs VHH-65, VHH-74, VHH-62 and VHH-56, disclosed in WO 2022/136647.

In certain embodiments of the methods comprising use of a therapeutic anti-CCR8 mAb that binds to an epitope outside the N-terminal domain of hCCR8, the non-competing mAb is an Ab that binds to an epitope in the N-terminal domain and does not compete for binding to hCCR8 with a mAb that binds to an epitope outside the N-terminal domain. Examples of such Abs that bind to an epitope in the N-terminal domain include Clone L263G8 (BioLegend) and mAb 433H disclosed in WO 2007/044756 (and marketed by BD Biosciences, https://www.bdbiosciences.com/content/bdb/paths/generate-tds-document.us.566899.pdf).

This disclosure also provides a method for measuring RO of a cell membrane-bound CCR8 receptor to which a therapeutic Treg-depleting anti-CCR8 Ab binds, as exemplified in Example 6. This method comprises: (a) adding to background whole blood samples, previously exposed to various concentrations of the therapeutic mAb, a saturating concentration of the therapeutic mAb; (b) incubating aliquots of free (i.e., blood samples from indirect/total experiment tube), bound (i.e., blood samples from direct experiment tubes) or fluorescence minus one [FMO](i.e., blood sample control tube that is stained with all the Abs except the anti-Id Ab for direct RO format) samples with buffer not containing the therapeutic mAb; (c) staining the samples with a core panel of Abs to identify basic T cell markers, T cell differentiation markers, and Treg markers; (d) adding to the core cocktail panel (i) for the direct/total RO assay, an anti-idiotypic Ab for detecting bound CCR8 receptor, or (ii) for the indirect/total assay, two additional Abs comprising an allophycocyanin (APC)-conjugated anti-hCCR8 Ab that competes with the therapeutic Ab, for detecting free CCR8 receptors, and an anti-hCCR8 Ab that does not compete with the therapeutic Ab, for detecting total CCR8 receptors; (e) lysing the red blood cells (to clear the samples while keeping the target cells intact) and analyzing the cleared samples by flow cytometry; and (f) determining the % RO for each concentration of the therapeutic Ab using the formula:

% RO=100×[1−((free postdose/free predose)/(total postdose/total predose))].

In certain embodiments of this method, the therapeutic Ab binds specifically to an epitope in the N-terminal domain of hCCR8. In certain other embodiments, the therapeutic Ab is one of the Abs described in WO 2021/194942 that bind specifically to an epitope in the N-terminal domain of hCCR8. In further embodiments, the therapeutic Ab comprises the 6 CDRs (SEQ ID NOs. 53-58 herein), or the heavy and/or light variable regions (SEQ ID NO: 9 and/or 16 herein), of mAb 4A19 described in WO 2021/194942. In certain preferred embodiments, the therapeutic Ab is mAb 4A19 which comprises the heavy and/or light chains (SEQ ID NO: 65 and/or 72) of mAb 4A19.

In certain embodiments, the exposure of whole blood samples to various doses of the therapeutic mAb occurs when the therapeutic Ab is administered to a subject, e.g., during a clinical trial or in a course of treatment with an anti-CCR8 mAb. In other embodiments, the whole blood samples are exposed to different concentrations of the therapeutic Ab in vitro to mimic different drug dosing in patients.

In certain embodiments, the basic T cell markers used in the core panel of markers include UV-excitable, fluorescent dye-conjugated anti-human CD3, anti-human CD4 and/or anti-human CD8 Abs. In certain other embodiments, the T cell differentiation marker includes a fluorescently labeled anti-human CD45RA Ab. In certain other embodiments, the Treg markers include fluorescently labeled anti-human CD25, anti-human CD127 and/or anti-human CCR4 Abs. In yet further embodiments, the competing anti-hCCR8 Ab added to the panel for the indirect/total assay is fluorescently labeled mAb 433H (BD Biosciences) for detecting free CCR8 receptors, and the non-competing Ab for detecting total CCR8 receptors is fluorescently labeled mAb 21C17.

As described in Example 6, CCR8 engagement measured using RO assays in both the direct and indirect assay format showed that the hCCR8 target expressed on a cell surface was specifically engaged by the therapeutic mAb 4A19. This engagement of the receptor by the therapeutic Ab is consistent with the underlying mechanism of action expected for anti-CCR8 immunotherapy which requires that the anti-CCR8 Ab bind to CCR8 on Tregs and mediate the depletion of these immunosuppressive Tregs (see Example 8) by processes including ADCC, ADCP, and/or CDC. The RO assay is also quantitative and further demonstrates that the % RO on CCR8⁺ Tregs correlates with increasing dosage of the therapeutic Ab. These % RO data can inform the selection of appropriate dosage regimens, i.e., dosing amount and frequency, to optimize efficacy, as well as minimizing potential safety issues by avoiding dosages in excess of what are required for effective receptor occupancy.

This disclosure also provides a method for measuring a depletion in the frequency of CCR8-expressing cells in the peripheral blood of a subject undergoing treatment with an anti-CCR8 mAb, the method comprising: (a) determining a baseline level of the frequency of CCR8-expressing cells in a first sample of whole blood or PBMCs from the subject; (b) administering a treatment of an anti-CCR8 mAb to the subject; and (c) determining the frequency of CCR8-expressing cells in a second sample of whole blood or PBMCs from the subject taken after administration of the anti-CCR8 mAb; wherein a decrease in the frequency of CCR8-expressing cells in the second sample indicates that the number of CCR8-expressing cells in the blood has been depleted. This method allows the depletion of CCR8⁺ Tregs in the peripheral blood of a subject being treated with an anti-CCR8 mAb to be monitored. Since anti-CCR8 Ab immunotherapy is premised on the Ab causing depletion of CCR8-expressing, immunosuppressive Tregs, measurement of Treg depletion mediated by administration of an anti-CCR8 therapeutic Ab may allow the efficacy of treatment with an anti-CCR8 therapeutic Ab to be evaluated even before clinical indications of efficacy are manifested. Because CCR8⁺ Tregs represent a very small percentage, about 0.5-2% of PBMCs, the availability of anti-CCR8 Abs that do not compete with a therapeutic anti-CCR8 Ab for binding to CCR8, such as the anti-CCR8 mAbs of the invention that do not compete with a therapeutic Ab binding to the N-terminal domain of hCCR8, are important for detecting and quantifying these low levels of CCR8⁺ cells as they are not competing for binding to an N-terminal epitope to which a therapeutic mAb has already bound.

This disclosure also provides a method for measuring a depletion in the number of tumor-infiltrating CCR8-expressing Tregs in a subject undergoing treatment with an anti-CCR8 mAb, the method comprising: (a) determining a baseline level of the frequency of CCR8-expressing Tregs in a first sample of test tissue in or from the subject, the test tissue comprising tumor cells and tumor-infiltrating Tregs; (b) administering a treatment of an anti-CCR8 mAb to the subject; and (c) determining the frequency of CCR8-expressing Tregs, in a second sample of test tissue in or from a subject taken during or after the treatment; wherein a decrease in the frequency of CCR8-expressing Tregs in the second sample of the test tissue indicates that the number of Tregs in the test tissue has been depleted. This method provides a means of monitoring the depletion of tumor-infiltrating Tregs in the tumor environment of a subject. Since the expected mechanism of action of anti-CCR8 immunotherapy is that an effective anti-CCR8 Ab causes depletion of CCR8-expressing, immunosuppressive Tregs, measurement of Treg depletion in the tumor environment mediated by administration of an anti-CCR8 therapeutic Ab allows the likely effectiveness of treatment with an anti-CCR8 therapeutic Ab to be evaluated even before clinical indications of efficacy are manifested.

In certain embodiments of this method, a depletion in the number of tumor-infiltrating Tregs in the test tissue indicates an enhancement in an immune response in the subject, and/or efficacy in treating a disease that is treatable by an enhanced immune response. In certain embodiments, the treatment administered to the subject is a treatment for cancer. In further embodiments, the treatment for cancer comprises administration of a therapeutic anti-CCR8 Ab or an antigen-binding portion thereof.

In certain other embodiments, the anti-CCR8 antibody treatment administered to the subject is a treatment for an infectious disease.

CCR8 is expressed on 60-80% of all skin T cells, not only on Tregs. Accordingly, the present method can also be adapted for measuring a depletion in the number of skin T cells in a subject undergoing treatment, the method comprising: (a) determining a baseline level of the frequency of CCR8-expressing skin T cells in a first sample of test tissue in or taken from a subject, the test tissue comprising skin T cells; (b) administering a treatment of an anti-CCR8 mAb to the subject; and (c) determining the frequency of CCR8-expressing skin T cells in a second sample of test tissue in or taken from a subject during or after the treatment; wherein a decrease in the frequency of CCR8-expressing skin T cells in the second test tissue indicates that the number of skin T cells in the test tissue has been depleted.

Because Tregs are immunosuppressive, it is expected that subjects who express high levels of Tregs, i.e., high levels of CCR8-expressing T cells, in the tumor environment would most benefit from treatment with a Treg-depleting Ab, and therefore, would be suitable candidates for immunotherapy with a therapeutic Treg-depleting Ab. Accordingly, this disclosure also provides a method for predicting the effectiveness of a therapeutic Treg-depleting Ab or an antigen-binding portion thereof in treating cancer in a subject, which method comprises: (a) determining the frequency of CCR8-expressing Tregs in a test tissue in or taken from the subject, the test tissue comprising tumor cells and tumor-infiltrating Tregs; (b) comparing the frequency of CCR8-expressing Tregs with a predetermined threshold value; and (c) predicting the effectiveness of the therapeutic Treg-depleting Ab or antigen-binding portion thereof, wherein a frequency of CCR8-expressing Tregs higher than the threshold value indicates that the therapeutic Ab or antigen-binding portion thereof will be effective in treating the subject, and wherein a frequency of CCR8-expressing Tregs lower than the threshold value indicates that the therapeutic Ab or antigen-binding portion thereof will not be effective in treating the subject. In certain preferred embodiments of this method, the Treg-depleting Ab is an anti-CCR8 Ab. In certain other embodiments, the Treg-depleting Ab is an anti-CTLA-4 Ab. In further embodiments, the Treg-depleting Ab is an anti-CCR4 or an anti-CD25 Ab.

This disclosure also provides a method for predicting the effectiveness of a therapeutic Treg-depleting Ab or an antigen-binding portion thereof in treating cancer in a subject, which method comprises: (a) determining the frequency of CCR8-expressing Tregs in a test tissue in or taken from the subject, the test tissue comprising tumor cells and tumor-infiltrating Tregs; (b) administering the therapeutic Treg-depleting Ab or antigen-binding portion thereof the subject; (c) determining whether there is a decrease in the frequency of CCR8-expressing Tregs after administration of the therapeutic Treg-depleting Ab or antigen-binding portion thereof, and (d) predicting that the therapeutic Treg-depleting Ab or antigen-binding portion thereof will be effective in treating cancer in the subject if the decrease in the frequency of CCR8-expressing Tregs exceeds a predetermined threshold value, or (e) predicting that the therapeutic Treg-depleting Ab or antigen-binding portion thereof will not be effective in treating cancer in the subject if the decrease in the frequency of CCR8-expressing Tregs is less than a predetermined threshold value. In certain preferred embodiments of this method, the Treg-depleting Ab is an anti-CCR8 Ab. In certain other embodiments, the Treg-depleting Ab is an anti-CTLA-4 Ab. In further embodiments, the Treg-depleting Ab is an anti-CCR4 or an anti-CD25 Ab.

The disclosure also provides a method for selecting a subject afflicted with a cancer as a suitable candidate for cancer immunotherapy with a therapeutic Treg-depleting Ab or an antigen-binding portion thereof, which method comprises: (a) determining the frequency of CCR8-expressing Tregs in a test tissue in or taken from a subject, the test tissue comprising tumor cells and tumor-infiltrating Tregs; (b) comparing the frequency of CCR8-expressing Tregs with a predetermined threshold value; and (c) selecting the subject as a suitable candidate for immunotherapy with the therapeutic Treg-depleting Ab or antigen-binding portion thereof based on an assessment that the frequency of CCR8-expressing Tregs in the test tissue exceeds the predetermined threshold value. In certain preferred embodiments of this method, the Treg-depleting Ab is an anti-CCR8 Ab. In certain other embodiments, the Treg-depleting Ab is an anti-CTLA-4 Ab. In further embodiments, the Treg-depleting Ab is an anti-CCR4 or an anti-CD25 Ab.

The disclosure also provides a method for selecting a subject afflicted with a cancer as a suitable candidate for cancer immunotherapy with a therapeutic Treg-depleting Ab or an antigen-binding portion thereof, which method comprises: (a) determining the frequency of CCR8-expressing Tregs in a test tissue in or taken from the subject, the test tissue comprising tumor cells and tumor-infiltrating Tregs; (b) administering the therapeutic Treg-depleting Ab or antigen-binding portion thereof the subject; (c) determining whether there is a decrease in the frequency of CCR8-expressing Tregs after administration of the therapeutic Treg-depleting Ab or antigen-binding portion thereof, and (d) selecting the subject as a suitable candidate for immunotherapy with the therapeutic Treg-depleting Ab or antigen-binding portion thereof based on an assessment that a decrease in the frequency of CCR8-expressing Tregs exceeds a predetermined threshold value. In certain preferred embodiments of this method, the Treg-depleting Ab is an anti-CCR8 Ab. In certain other embodiments, the Treg-depleting Ab is an anti-CTLA-4 Ab. In further embodiments, the Treg-depleting Ab is an anti-CCR4 or an anti-CD25 Ab.

This disclosure provides a method for treating a cancer in a subject, which method comprises: (a) selecting a subject that is a suitable candidate for cancer immunotherapy with a therapeutic Treg-depleting Ab or an antigen-binding portion thereof, the selecting comprising: (i) determining the frequency of CCR8-expressing Tregs in a test tissue in or taken from a subject, the test tissue comprising tumor cells and tumor-infiltrating Tregs; (ii) comparing the frequency of CCR8-expressing Tregs with a predetermined threshold value; and (iii) selecting the subject as a suitable candidate for cancer immunotherapy with the therapeutic Treg-depleting Ab or antigen-binding portion thereof based on an assessment that the frequency of CCR8-expressing Tregs in the test tissue exceeds the predetermined threshold value; and (b) administering to the selected subject a composition comprising a therapeutically effective amount of the therapeutic Treg-depleting Ab or antigen-binding portion thereof. In certain preferred embodiments of this method, the Treg-depleting Ab is an anti-CCR8 Ab.

This disclosure further provides a method for treating a cancer in a subject, which method comprises: (a) selecting a subject that is a suitable candidate for cancer immunotherapy with a therapeutic Treg-depleting Ab or an antigen-binding portion thereof, the selecting comprising: (i) determining the frequency of CCR8-expressing Tregs in a test tissue in or taken from the subject, the test tissue comprising tumor cells and tumor-infiltrating Tregs; (ii) administering the therapeutic Treg-depleting Ab or antigen-binding portion thereof to the subject; (iii) determining whether there is a decrease in the frequency of CCR8-expressing Tregs after administration of the therapeutic Treg-depleting Ab or antigen-binding portion thereof, and (iv) selecting the subject as a suitable candidate for immunotherapy with the therapeutic based on an assessment that a decrease in the frequency of CCR8-expressing Tregs exceeds a predetermined threshold value; and (b) administering to the selected subject a composition comprising a therapeutically effective amount of the therapeutic Treg-depleting Ab or antigen-binding portion thereof. In certain preferred embodiments of this method, the Treg-depleting Ab is an anti-CCR8 Ab.

The disclosure also provides a method for treating a cancer in a subject, which method comprises (a) selecting a subject that is not a suitable candidate for cancer immunotherapy with a therapeutic Treg-depleting Ab or an antigen-binding portion thereof, the selecting comprising: (i) determining the frequency of CCR8-expressing Tregs in a test tissue in or from the subject, the test tissue comprising tumor cells and tumor-infiltrating Tregs; (ii) comparing the frequency of CCR8-expressing Tregs with a predetermined threshold value; and (iii) selecting the subject as not suitable for immunotherapy with the therapeutic Treg-depleting Ab or antigen-binding portion thereof based on an assessment that the frequency of CCR8-expressing Tregs in cells of the test tissue is less than the predetermined threshold value; and (b) administering a standard-of-care therapeutic other than the therapeutic anti-CCR8 Ab or antigen-binding portion thereof to the selected subject.

The disclosure also provides a method for treating cancer in a subject, which method comprises (a) selecting a subject that is not a suitable candidate for cancer immunotherapy with a therapeutic Treg-depleting Ab or an antigen-binding portion thereof, the selecting comprising: (i) determining the frequency of CCR8-expressing Tregs in a test tissue in or taken from the subject, the test tissue comprising tumor cells and tumor-infiltrating Tregs; (ii) administering the therapeutic Treg-depleting Ab or antigen-binding portion thereof the subject; (iii) determining whether there is a decrease in the frequency of CCR8-expressing Tregs after administration of the therapeutic Treg-depleting Ab or antigen-binding portion thereof, and (iv) selecting the subject as not suitable for immunotherapy with the therapeutic Treg-depleting Ab or antigen-binding portion thereof based on an assessment that the decrease in the frequency of CCR8-expressing Tregs is less than a predetermined threshold value; and (b) administering to the selected subject a standard-of-care therapeutic other than the therapeutic anti-CCR8 Ab or antigen-binding portion thereof. In certain preferred embodiments of this method, the Treg-depleting Ab is an anti-CCR8 Ab.

The disclosure also provides a method for treating cancer in a subject, which method comprises administering to the subject a composition comprising a therapeutically effective amount of a therapeutic Treg-depleting Ab or an antigen-binding portion thereof, the subject having been selected on the basis that the frequency of CCR8-expressing Tregs in cells of a test tissue in or from the subject, the test tissue comprising tumor cells and tumor-infiltrating Tregs, was determined to exceed a predetermined threshold level.

The disclosure also provides a method for treating cancer in a subject, which method comprises administering to the subject a composition comprising a therapeutically effective amount of a therapeutic Treg-depleting Ab or an antigen-binding portion thereof, the subject having been selected on the basis that the therapeutic Treg-depleting Ab or antigen-binding portion thereof causes a decrease in the frequency of CCR8-expressing Tregs that exceeds a predetermined threshold value.

The disclosure further provides a method for treating cancer in a subject, which method comprises administering to the subject a standard-of-care treatment other than a therapeutic Treg-depleting Ab or an antigen-binding portion thereof, the subject having been selected on the basis that the frequency of CCR8-expressing Tregs in a test tissue in or from the subject, the test tissue comprising tumor cells and tumor-infiltrating Tregs, was determined to be less than a predetermined threshold level.

The disclosure further provides a method for treating cancer in a subject, which method comprises administering to the subject a standard-of-care treatment other than a therapeutic Treg-depleting Ab or an antigen-binding portion thereof, the subject having been selected on the basis that the therapeutic Treg-depleting Ab or antigen-binding portion thereof causes a decrease in the frequency of CCR8-expressing Tregs that is less than a predetermined threshold level.

In certain preferred embodiments of any of the above methods for treating a cancer in a subject, the therapeutic Treg-depleting Ab is an anti-CCR8 Ab. In further preferred embodiments, the Treg-depleting Ab or antigen-binding portion thereof is a mAb comprising the 6 CDRs, the heavy and light chain variable regions, or the heavy and light chains, of the mAb designated 4A19. In certain other embodiments, the Treg-depleting Ab or antigen-binding portion thereof is 4A19, 18Y12, 8D55, 10R3, 14S15, or 14S15h, or an antigen-binding portion thereof. In certain preferred embodiments, the Treg-depleting Ab is mAb 4A19. In certain other embodiments, the Treg-depleting Ab is an anti-CTLA-4 Ab. In further embodiments, the Treg-depleting Ab is an anti-CCR4 or an anti-CD25 Ab.

In certain situations, for example where there are limitations on the availability of tissue samples from tumor biopsies or where it is desirable to predict or monitor efficacy before clinical indications are manifested in the tumor, it may be quicker, more efficient, and/or more practical to measure the frequency of CCR8-expressing cells, and the depletion of CCR8-expressing cells induced by administration of a Treg-depleting treatment, using blood or PBMC samples rather than tumor samples. As disclosed herein, the non-competing anti-CCR8 mAbs of the invention enable the quantification of CCR8-expressing cells and depletion of CCR8-expressing cells in whole blood or PBMCs, notwithstanding the very low frequencies of such cells in peripheral blood. Accordingly, in any of the methods described above for predicting the effectiveness of a therapeutic Treg-depleting Ab in treating cancer in a patient, selecting a cancer patient as a suitable candidate for cancer immunotherapy with a therapeutic Treg-depleting Ab, and/or for treating such patient with a therapeutic Treg-depleting Ab, the steps of determining the frequency of CCR8-expressing Tregs, and/or determining whether there is a decrease in the frequency of CCR8-expressing Tregs after administration of the therapeutic Treg-depleting Ab, are performed using blood or PBMC samples in or taken from the subject.

In certain embodiments, the Treg-depleting Ab is administered to the subject in combination with a therapeutically effective amount of an additional therapeutic agent for treating a cancer. In certain embodiments, the additional anti-cancer agent is a small molecule, a polypeptide, an antibody, an immunoregulatory agent, a chemotherapeutic agent, an agent for targeted therapy, or any combination thereof. In certain embodiments, the immunotherapy comprises an agent that reduces inhibition, or increases stimulation, of the immune system. In certain preferred embodiments, the additional therapeutic agent is a compound that reduces inhibition, or increases stimulation, of the immune system. Such an additional therapeutic agent may be, for example, a small-molecule compound, a macrocyclic peptide, a fusion protein, or an Ab, e.g., a mAb. In certain embodiments, the additional therapeutic agent that reduces inhibition of the immune system is an antagonistic agent, such as an antagonistic mAb, that binds specifically to immune-inhibitory receptors, for example, Programmed Death-1 (PD-1), Programmed Death Ligand-1 (PD-L1), Cytotoxic T-Lymphocyte Antigen-4 (CTLA-4), Lymphocyte Activation Gene-3 (LAG-3), B and T lymphocyte Attenuator (BTLA), T cell Immunoglobulin and Mucin domain-3 (TIM-3), Killer Immunoglobulin-like Receptor (KIR), Killer cell Lectin-like Receptor G1 (KLRG-1), Adenosine A2a Receptor (A2aR), T Cell Immunoreceptor with Ig and ITIM Domains (TIGIT), V-domain Ig Suppressor of T cell activation (VISTA), proto-oncogene tyrosine-protein kinase MER (MerTK), Natural Killer Cell Receptor 2B4 (CD244), or CD160.

In certain preferred embodiments, the additional therapeutic agent is an antagonistic Ab or antigen-binding portion thereof that binds specifically to PD-1. In further embodiments, the Ab that binds specifically to PD-1 is chosen from nivolumab, pembrolizumab, cemiplimab, spartalizumab, camrelizumab, sintilimab, tislelizumab, toripalimab, dostarlimab, retifanlimab, zimberelimab, pimivalimab, serplulimab, vopratelimab, and acrixolimab. In certain preferred embodiments, the Ab is chosen from nivolumab and pembrolizumab.

In other preferred embodiments, the additional therapeutic agent is an antagonistic agent, such as an Ab or antigen-binding portion thereof, that binds specifically to PD-L1. In further embodiments, the agent that binds specifically to PD-L1 is chosen from atezolizumab, durvalumab, avelumab, envafolimab, cosibelimab (CK-301), BMS-936559, BMS-986189, CS-1001, SHR-1316, CBT-502, BGB-A333, KN035, AUNP12, and CA-170. In certain preferred embodiments, the agent is an Ab chosen from atezolizumab, durvalumab and avelumab.

In other preferred embodiments, the additional therapeutic agent is an antagonistic Ab or antigen-binding portion thereof that binds specifically to CTLA-4. In further embodiments, the Ab that binds specifically to CTLA-4 is ipilimumab or tremelimumab. In certain preferred embodiments, the Ab ipilimumab.

In other preferred embodiments, the additional therapeutic agent is an antagonistic agent that binds specifically to LAG-3. In further embodiments, the agent that binds specifically to LAG-3 is selected from Abs relatlimab, favezelimab, tiragolumab, fianlimab, or tebotelimab, or the soluble protein eftilagimod alpha. In certain preferred embodiments, the Ab is relatlimab.

In certain embodiments, the additional therapeutic agent that increases stimulation of the immune system is an agonistic agent, such as an agonistic mAb, that binds specifically to immune-stimulatory receptors, for example, Inducible T cell Co-Stimulator (ICOS), CD137 (4-1BB), CD134 (OX40), CD27, Glucocorticoid-Induced TNFR-Related protein (GITR), or HerpesVirus Entry Mediator (HVEM).

In certain embodiments of the above methods, determining the level of expression of CCR8 in the test tissue comprises assessing the level of expression of CCR8 on the surface of Tregs in the test tissue. In certain other embodiments, determining the level of expression of CCR8 in the test tissue comprises assessing the proportion of Tregs in the test tissue that express CCR8 on the Treg cell surface.

In further embodiments, the level of expression of CCR8 in the test tissue in the subject is determined by an in vivo method. In yet further embodiments, the in vivo method comprises PET tracing, for example, using an anti-CCR8 Ab of the invention that binds to an epitope of CCR8 outside the N-terminal domain.

In other embodiments, the level of expression of CCR8 is determined ex vivo in a test tissue sample obtained from the subject. For example, in certain embodiments of the ex vivo methods, the level of expression of CCR8 is determined by immunohistochemistry (IHC), flow cytometry, or mass spectrometry-coupled flow cytometry, using a labeled anti-CCR8 Ab of the invention or an antigen-binding portion thereof to bind to CCR8 expressed on the surface of a cell in the tissue. In certain embodiments, the IHC is performed on fresh frozen tissue or formalin-fixed paraffin-embedded (FFPE) tissue. In certain preferred embodiments, determining the level of expression of CCR8 using an anti-CCR8 Ab of the invention is not affected by the binding of a therapeutic Ab or an antigen-binding portion thereof to the N-terminal domain of CCR8.

In certain embodiments of any of the methods described above, the therapeutic anti-CCR8 Ab or antigen-binding portion thereof comprises:
(a) a heavy chain variable region CDR1 comprising consecutively linked amino acids having the sequence set forth as SEQ ID NO: 53; a heavy chain variable region CDR2 comprising consecutively linked amino acids having the sequence set forth as SEQ ID NO: 54; a heavy chain variable region CDR3 comprising consecutively linked amino acids having the sequence set forth as SEQ ID NO: 55; a light chain variable region CDR1 comprising consecutively linked amino acids having the sequence set forth as SEQ ID NO: 56; a light chain variable region CDR2 comprising consecutively linked amino acids having the sequence set forth as SEQ ID NO: 57; and a light chain variable region CDR3 comprising consecutively linked amino acids having the sequence set forth as SEQ ID NO: 58;
(b) a V_{H} comprising consecutively linked amino acids having the sequence set forth as SEQ ID NO: 9 and a V_{L} comprising consecutively linked amino acids having the sequence set forth as SEQ ID NO: 16; and/or
(c) a heavy chain comprising consecutively linked amino acids having the sequence set forth as SEQ ID NO: 65 and a light chain comprising consecutively linked amino acids having the sequence set forth as SEQ ID NO: 72.

In certain other embodiments, the therapeutic anti-CCR8 Ab or antigen-binding portion thereof is the Ab designated in WO 2021/194942 as 4A19, 18Y12, 8D55, 10R3, 14S15, or 14S15h, or an antigen-binding portion thereof.

In certain embodiments of the methods described herein requiring a therapeutic anti-CCR8 Ab or an antigen-binding portion thereof, the therapeutic Ab or antigen-binding portion thereof is an mAb comprising the 6 CDRs, the heavy and light chain variable regions, and/or the heavy and light chains, of the mAbs designated 4A19, 18Y12, 8D55, 10R3, 14S15, or 14S15h in WO 2021/194942. In further embodiments, the therapeutic Ab or antigen-binding portion thereof is 4A19, 18Y12, 8D55, 10R3, 14S15, or 14S15h or an antigen-binding portion thereof. In certain preferred embodiments, the therapeutic Ab is 4A19.

In certain embodiments of such methods requiring an anti-CCR8 Ab that does not compete with the therapeutic Ab for binding to CCR8, the non-competing Ab is an mAb comprising the 6 CDRs, the heavy and light chain variable regions, and/or the heavy and light chains, of the mAbs designated herein as 23A14, 21C17, 22B13, 25T40, 28P3, and 33H18. In certain preferred embodiments, the non-competing Ab is 21C17.

In certain preferred embodiments of any of these methods, the subject is a human.

### Cancers Treatable by Depletion of Tregs

Immuno-oncology, which relies on using the practically infinite flexibility of the immune system to attack and destroy cancer cells, is applicable to treating a very broad range of cancers (see, e.g., Yao et al., 2013; Callahan et al., 2016; Pianko et al., 2017; Farkona et al., 2016; Kamta et al., 2017; Drugs.com—Opdivo Approval History: https://www.drugs.com/history/opdivo.html). For example, the anti-PD-1 Ab, nivolumab, has been shown to be effective in treating many different types of cancers (see, e.g., Brahmer et al., 2015; Guo et al., 2017; Pianko et al., 2017; WO 2013/173223; Drugs.com—Opdivo Approval History), and is currently undergoing clinical trials in multiple solid and hematological cancers. Similarly, anti-PD-L1 drugs such as atezolizumab (TECENTRIQ®), durvalumab (vIMFINZI®) and avelumab (BAVENCIO®) have been gaining approvals in a variety of indications. Accordingly, immunotherapeutic methods for treating cancer employing depletion of Tregs, for example CCR8-mediated depletion of tumor-infiltrating Tregs as disclosed herein, are applicable to treating a wide variety of both solid and liquid tumors.

For example, in certain embodiments of the Treg depletion therapeutic methods for treating a cancer, in which Abs of the invention are used to monitor changes in numbers of Tregs, the cancer is a solid tumor.

Based on the demonstration of effective treatment of different cancers with anti-CCR8 in mouse models (see WO 2021/194942), certain tumor types are expected to be particularly amenable to treatment with an anti-CCR8 Ab. Accordingly, in certain embodiments of the methods disclosed herein, the solid tumor is a cancer chosen from colon adenocarcinoma, bladder carcinoma, mammary carcinoma, and fibrosarcoma.

In certain embodiments, based on the relatively high expression of CCR8 and CD8A and the high CCR8/CD8A ratio in CCR8⁺ Tregs identified by single-cell RNA-seq analysis, the solid tumor is a cancer chosen from head and neck squamous cell carcinoma (HNSC), lung adenocarcinoma (LUAD), stomach adenocarcinoma (STAD), lung squamous cell carcinoma (LUSC), pancreatic adenocarcinoma (PAAD), rectum adenocarcinoma (READ), esophageal carcinoma (ESCA), breast invasive carcinoma (BRCA), colon adenocarcinoma (COAD) and cervical squamous cell carcinoma and endocervical adenocarcinoma (CESC).

High levels of CCR8 expression in certain tumors may be identified by IHC in FFPE tissue samples, and tumors expressing high levels of CCR8 are more likely to respond to treatment with an anti-CCR8 Ab. Accordingly, in certain embodiments, the solid tumor is a cancer chosen, based on CCR8 expression levels, from head and neck squamous cell carcinoma (HNSCC; also referred to herein as squamous cell carcinoma of the head and neck [SCCHN]), cervical cancer, colorectal cancer (CRC), non-small cell lung cancer-squamous cell carcinoma (NSCLC-SCC), NSCLC-adenocarcinoma (NSCLC-ADC), pancreatic, gastric, bladder, and breast cancers.

For a phase 1/2 clinical trial of the 4A19 anti-CCR8 mAb administered as monotherapy and in combination with the anti-PD-1 mAb, nivolumab, certain solid tumors were selected based on the demonstrated efficacy of anti-CCR8 in mouse tumor models, RNA expression of CCR8 and CD8A in the tumor types represented in The Cancer Genome Atlas (National Cancer Institute, 2021), tumor types having relatively high expression of CCR8 and enrichment for CD8A expression, and levels of CCR8 expression in different tumor types or subtypes as measured by IHC. Based on the tumor types selected for the clinical trial, in certain embodiments of the disclosed methods the solid tumor is a cancer chosen from non-small cell lung cancer (NSCLC), SCCHN, Microsatellite Stable colorectal cancer (MSS-CRC), gastric/gastroesophageal (GE) junction adenocarcinoma, cervical cancer (squamous cell carcinoma [SCC] or adenocarcinoma), renal cell carcinoma (RCC), urothelial carcinoma (UC), pancreatic ductal adenocarcinoma (PDAC), melanoma, ovarian cancer (OC), and triple-negative breast cancer (TNBC).

In further embodiments, the solid tumor is a cancer selected from squamous cell carcinoma, small cell lung cancer (SCLC), NSCLC, squamous NSCLC, non-squamous NSCLC, head and neck cancer, breast cancer, cancer of the esophagus, gastric cancer, gastrointestinal cancer, cancer of the small intestine, liver cancer, hepatocellular carcinoma (HCC), pancreatic cancer (PAC), kidney cancer, RCC, bladder cancer, cancer of the urethra, cancer of the ureter, colorectal cancer (CRC), colon cancer, colon carcinoma, cancer of the anal region, endometrial cancer, prostate cancer, a fibrosarcoma, neuroblastoma, glioma, glioblastoma, germ cell tumor, pediatric sarcoma, sinonasal natural killer, melanoma, skin cancer, bone cancer, cervical cancer, uterine cancer, carcinoma of the endometrium, carcinoma of the fallopian tubes, ovarian cancer, carcinoma of the cervix, carcinoma of the vagina, carcinoma of the vulva, testicular cancer, cancer of the endocrine system, thyroid cancer, cancer of the parathyroid gland, cancer of the adrenal gland, sarcoma of soft tissue, cancer of the penis, carcinoma of the renal pelvis, neoplasm of the central nervous system (CNS), primary CNS lymphoma, tumor angiogenesis, spinal axis tumor, brain cancer, brain stem glioma, pituitary adenoma, Kaposi's sarcoma, epidermoid cancer, squamous cell cancer, solid tumors of childhood, environmentally-induced cancers, virus-related cancers, and cancers of viral origin. In certain embodiments, the cancer is an advanced, unresectable, metastatic, refractory cancer, or recurrent cancer, or any combination thereof.

In certain embodiments of the disclosed methods, the cancer is a hematological malignancy. Hematological malignancies include liquid tumors derived from either of the two major blood cell lineages, i.e., the myeloid cell line (which produces granulocytes, erythrocytes, thrombocytes, macrophages and mast cells) or the lymphoid cell line (which produces B, T, NK and plasma cells), including all types of leukemias, lymphomas, and myelomas.

TARGET (Therapeutically Applicable Research to Generate Effective Treatments, https://ocg.cancer.gov/programs/target) analysis indicated that, among hematological malignancies examined, follicular lymphoma (FL) and acute lymphocytic leukemia and lymphoma were found to have the highest relative expression of CCR8 and should be prioritized for treatment with an anti-CCR8 mAb (see WO 2021/194942). Thus, in certain embodiments of the present therapeutic methods, the hematological malignancy is FL or acute lymphocytic leukemia and lymphoma.

In certain other embodiments, the hematological malignancy is a cancer selected from acute, chronic, lymphocytic (lymphoblastic) and/or myelogenous leukemias, such as acute lymphoblastic leukemia (ALL), acute myelogenous leukemia (AML), chronic lymphocytic leukemia (CLL), and chronic myelogenous leukemia (CML); lymphomas, such as Hodgkin's lymphoma (HL), non-Hodgkin's lymphomas (NHLs), of which about 85% are B cell lymphomas, including diffuse large B-cell lymphoma (DLBCL), FL, CLL/small lymphocytic lymphoma (SLL), mantle cell lymphoma, marginal zone B-cell lymphomas (mucosa-associated lymphoid tissue (MALT) lymphoma, nodal marginal zone B-cell lymphoma, and splenic marginal zone B-cell lymphoma), Burkitt lymphoma, lymphoplasmacytoid lymphoma (LPL; also known as Waldenstrom's macroglobulinemia (WM)), hairy cell lymphoma, and primary central nervous system (CNS) lymphoma, NHLs that are T cell lymphomas, including precursor T-lymphoblastic lymphoma/leukemia, T-lymphoblastic lymphoma/leukemia (T-Lbly/T-ALL), peripheral T-cell lymphomas such as cutaneous T-cell lymphoma (CTLC, i.e., mycosis fungoides, Sezary syndrome and others), adult T-cell lymphoma/leukemia, angioimmunoblastic T-cell lymphoma, extranodal natural killer/T-cell lymphoma nasal type, enteropathy-associated intestinal T-cell lymphoma (EATL), anaplastic large-cell lymphoma (ALCL), and peripheral T-cell lymphoma unspecified, acute myeloid lymphoma, lymphoplasmacytoid lymphoma, monocytoid B cell lymphoma, angiocentric lymphoma, intestinal T-cell lymphoma, primary mediastinal B-cell lymphoma, post-transplantation lymphoproliferative disorder, true histiocytic lymphoma, primary effusion lymphoma, diffuse histiocytic lymphoma (DHL), immunoblastic large cell lymphoma, and precursor B-lymphoblastic lymphoma; myelomas, such as multiple myeloma, smoldering myeloma (also called indolent myeloma), monoclonal gammopathy of undetermined significance (MGUS), solitary plasmocytoma, IgG myeloma, light chain myeloma, nonsecretory myeloma, and amyloidosis; and any combinations of said hematological malignancies.

The present methods are also applicable to treatment of advanced, metastatic, refractory and/or recurrent hematological malignancies, and any combinations of said hematological malignancies.

### Kits

Also within the scope of the present invention are kits comprising an anti-CCR8 Ab that binds specifically to an epitope located outside the N-terminal domain of hCCR8 and the other components required to carry out any of the methods disclosed herein. Kits typically include a label indicating the intended use of the contents of the kit and instructions for use. The term label includes any writing, or recorded material supplied on or with the kit, or which otherwise accompanies the kit. Accordingly, this disclosure provides a kit for monitoring and/or quantifying the number of Tregs in a subject, wherein the kit is used for measuring a depletion in the number of Tregs in a subject, predicting the effectiveness of a therapeutic Treg-depleting Ab in treating a cancer in a subject, selecting a subject afflicted with cancer as a suitable candidate for immunotherapy with a therapeutic Treg-depleting Ab, or treating a cancer in a subject with a therapeutic Treg-depleting Ab, or treating a cancer in a subject with a standard-of-care therapeutic other than a therapeutic Treg-depleting Ab, as described in the methods disclosed herein. In certain embodiments, the kit comprises (a) a mAb of the invention or an antigen-binding portion thereof that binds specifically to an epitope located outside the N-terminal domain of hCCR8; (b) optionally, for the treatment methods, a therapeutic Treg-depleting Ab or an antigen-binding portion thereof that binds to the N-terminal domain of hCCR8, or alternatively a standard-of-care therapeutic other than a therapeutic Treg-depleting Ab; and (c) instructions for using the mAb of the invention or portion thereof in any of the methods disclosed herein for measuring a depletion in the number of Tregs in a subject, predicting the effectiveness of a therapeutic Treg-depleting Ab or an antigen-binding portion thereof in treating a cancer in a subject, selecting a subject afflicted with cancer as a suitable candidate for immunotherapy with a therapeutic Treg-depleting Ab or an antigen-binding portion thereof, treating a cancer in a subject with a therapeutic Treg-depleting Ab or an antigen-binding portion thereof, or treating a cancer in a subject with a standard-of-care therapeutic other than a therapeutic Treg-depleting Ab or an antigen-binding portion thereof.

In certain preferred embodiments of the above kits, the therapeutic Treg-depleting Ab is an anti-CCR8 Ab. In further preferred embodiments, the Treg-depleting Ab is an anti-CCR8 Ab that binds to an epitope in the N-terminal domain of hCCR8. For such a Treg-depleting Ab, the kit comprises a mAb of the invention or an antigen-binding portion thereof that binds specifically to an epitope located outside the N-terminal domain of hCCR8. In certain variants, the kit comprises a Treg-depleting Ab which is an anti-CCR8 Ab that binds to an epitope outside the N-terminal domain of hCCR8, in which case the kit comprises a mAb that binds specifically to an epitope within the N-terminal domain of hCCR8.

In certain other embodiments, the Treg-depleting Ab is an anti-CTLA-4 Ab. In further embodiments, the Treg-depleting Ab is an anti-CCR4 or an anti-CD25 Ab. In certain other embodiments, the kit comprises an additional anti-cancer therapeutic agent or therapy which may be, for example, a small molecule agent, a polypeptide, an antibody, an immunoregulatory agent, a chemotherapeutic agent, an agent for targeted therapy, or a combination thereof. In certain preferred embodiments, the additional anti-cancer therapeutic agent is a checkpoint inhibitor, such as an anti-PD-1, anti-PD-L1, anti-CTLA-4 or anti-LAG-3 Ab, and/or a chemotherapeutic agent such as docetaxel.

In certain embodiments, the kit comprises (a) one or more dosages ranging from about 0.1 to about 20 mg/kg body weight of a therapeutic mAb or an antigen-binding portion thereof that binds specifically to CCR8; and optionally (b) one or more dosages of a checkpoint inhibitor such as about 3 mg/kg body weight or 200 to about 1600 mg of an anti-PD-1/anti-PD-L1 mAb or an antigen-binding portion thereof.

The present invention is further illustrated by the following examples which should not be construed as further limiting. The contents of all references cited throughout this application are expressly incorporated herein by reference.

### Example 1

### Generation of MAbs Against CCR8

Mouse or human anti-hCCR8 mAbs were generated by immunizing C57BL/6 mice or a strain of transgenic mice that expresses a human Ig repertoire, respectively, with hCCR8 immunogens.

Immunizations with CCR8 Antigens

To generate Abs against hCCR8, cohorts of 2-5 C57BL/6 mice or human Ig transgenic mice were immunized every 5-7 days via footpad or base of tail injections, receiving 8-12 immunizations over 7-10 weeks with material derived from hCCR8-overexpressing cells. In some cases, plasma membrane fractions were isolated from 2.5×10⁶ hCCR8-overexpressing BA/F3 cells using differential centrifugation to enrich for hCCR8 protein in the plasma membranes and reduce administration of non-CCR8 cell antigens. In other cases, detergent-stabilized proteoliposome material was derived from HEK293 cells overexpressing a chimeric hCCR8/hCCR5 protein, and also overexpressing the anti-hCCR8 Ab Clone L263G8 (BioLegend). Doses of 5 μg of proteoliposome were delivered with n-dodecyl-B-D-maltoside (DDM) detergent in phosphate-buffered saline (PBS). Separate injections of oil-in-water RIBI adjuvant (Sigma-Aldrich, St. Louis, MO) were administered at sites adjacent to antigen injections to enhance the immune response without destabilizing the proteoliposomes or denaturing the extracellular conformation of CCR8.

To monitor immune responses, titrated serum from retroorbital or tail bleeds was screened by flow cytometry and ELISA as described in Example 2, typically after 4-6 weeks of immunizations. Serum was screened for Ab binding to multiple CCR8-overexpressing cell lines, corresponding negative control cell lines not overexpressing CCR8, and the sulfated N-terminal peptide of CCR8 conjugated to bovine serum albumin (BSA). CCR8-specific and CCR8-nonspecific Ab responses were measured in each animal, and animals with sufficient titers of anti-CCR8 Ig were selected for final immunizations 6 and 3 days before sacrifice and tissue harvest to create hybridoma fusions.

### Generation of Hybridomas Producing MAbs to CCR8

Lymphoid organs targeted by the immunization strategies were collected. Most typically, popliteal, inguinal, and iliac lymph nodes from mice immunized via footpad and base of tail with CCR8 immunogens were used. Tissues were homogenized into cell suspensions and combined with an equal number of immortalized mouse myeloma cells derived from the P3X63AgU.1 cell line (ATCC CRL-1597). The combined cells were prepared at a density of 1×10⁷ cells/ml in a low conductivity Cytofusion Medium C (BTX, Holliston, MA). Hybridomas were generated using an electrofusion unit (BTX) to fuse Ab-secreting B cells with the immortalized myeloma cells. The resulting cells were grown in flat bottom 96-well cell culture plates using hypoxanthine- and thymidine-containing Medium E (StemCell Technologies, Cambridge, MA) supplemented with aminopterin (Sigma-Aldrich) for the selection of hybridomas.

### Example 2

### Screening and Selection of Anti-Human CCR8 MAbs

Screening for MAbs that Selectively Bind to Human CCR8

In order to generate mAbs that bind to hCCR8, regular mice or human Ig transgenic mice were immunized with antigens derived from hCCR8-overexpressing cells and hybridomas were generated as described in Example 1. After 10-13 days of culture and growth media replacement, hybridoma culture supernatants were collected from individual wells and screened to identify wells with secreted CCR8-specific Abs. All supernatants were initially screened against at least two cell lines, one overexpressing hCCR8 and a corresponding control cell line not overexpressing CCR8. Ab binding on cells was measured through image-based fluorometric microvolume assay technology (FMAT) screening.

Hybridomas from positive wells were transferred to 24-well plates with new culture media, allowed to grow for 2-3 days, then screened again by flow cytometry to confirm Ab binding to CCR8. Briefly, 75-100 μl of hybridoma culture supernatant and CCR8-overexpressing cells (such as CCR8-overexpressing CHO or 293F cells) or control cells (such as GFP-CHO or parental 293F) were co-incubated for 30-60 min, washed, and incubated with anti-mouse IgG Fc or anti-human IgG Fc secondary Ab conjugated to AF647, APC, or PE (Jackson ImmunoResearch, West Grove, PA). After incubation and washing, fluorescence was measured by flow cytometry.

Anti-CCR8 Ab-secreting hybridomas were subcloned once or twice to ensure monoclonality. Briefly, approximately 700 viable hybridoma cells were plated in 5 ml of semi-solid methylcellulose medium (StemCell Technologies) with AF488-conjugated anti-human or anti-mouse IgG Ab (Jackson ImmunoResearch) used to detect hybridoma-secreted IgG. After 7 days, hybridoma colonies arising from single cells were imaged using the ClonePix2 system (Molecular Devices, San Jose, CA). Single colonies with desirable properties ((including distance from other colonies, IgG secretion levels, colony size, and colony circularity) were picked to 96-well plate wells and cultured for 2-4 days. Culture supernatant was screened by flow cytometry as previously described to confirm CCR8 Ab binding. Stable hybridoma subclones were cultured in vitro to generate Abs for affinity purification and further characterization. DNA sequences encoding the Ab heavy and light chains were obtained by standard sequencing techniques (Sanger sequencing and next generation sequencing). Predicted masses from Ab amino acid sequences were compared to known purified Ab masses obtained via mass spectroscopy to ensure sequencing accuracy.

### Characterizing the Epitopes of Anti-hCCR8 Abs

To broadly characterize the epitopes of confirmed CCR8-specific Abs, hybridoma culture supernatants were screened by ELISA to measure binding to hCCR8 N-terminal peptides. Briefly, a BSA-conjugated peptide corresponding to the N-terminal 35 amino acid residues of hCCR8 (SEQ ID NO: 74; 2 μg/ml) representing the properly sulfated hCCR8 N-terminus was coated onto high-binding 96-well plates (Corning, Corning, NY) overnight at 4° C. Plates were blocked with BSA and washed, then 100 μl of culture supernatant were added for 30-60 min on a plate shaker. After washing, an appropriate anti-Fc secondary Ab conjugated to horseradish peroxidase (HRP) was added and left for 30-60 min. The plates were developed with ABTS or HRP substrate (SurModics, Eden Prairie, MN), and absorbance was measured at 405 or 650 nm on a Sunrise microplate reader (TECAN; Mannedorf, Switzerland).

Abs generated against hCCR8 were shown by ELISA to bind predominantly, but not exclusively, to the BSA-conjugated N-terminal peptide sequence with two sulfated tyrosine residues. Several flow cytometry-based Ab competition experiments showed that this predominant group of hCCR8 N-terminal binding Abs consistently blocked one another from simultaneously binding to CCR8 on cells. However, 6 mAbs (23A14, 21C17, 22B13, 25T40, 28P3, and 33H18) were identified which specifically bound to hCCR8 by flow cytometry but did not bind by ELISA to the BSA-conjugated CCR8 N-terminal peptide, suggesting they bound to an epitope on hCCR8 that was not located in the N-terminal domain. These 6 Abs were characterized further by flow cytometry-based, binding competition experiments.

### Example 3

### Anti-CCR8 MAb 21C17, but not Commercial Clone L263G8, Binds to MAb 4A19-Pretreated Tumor CD4 Tregs

PCT Publication No. WO 2021/194942 describes humanized and human mAbs that bind with high affinity to hCCR8 expressed on a cell surface and mediate the depletion of CCR8-expressing tumor-infiltrating Tregs by mechanisms including ADCC. One of these mAbs, designated 4A19, was shown to bind to an epitope in the N-terminal domain of CCR8 comprising residues 12-22 and including sulfated tyrosine-15 and tyrosine-17 residues.

A study was conducted to determine whether the binding of mAb 4A19 to hCCR8 blocked the binding of 21C17. Tissue from a gastric tumor was dissociated using a Tumor Dissociation Kit (Miltenyi Biotec, Sunnyvale, CA) in conjunction with a gentleMACS Dissociator (Miltenyi Biotec), and stored in liquid nitrogen. For this study, thawed cells were incubated for 15 min on ice with unlabeled mAb 4A19, unlabeled mAb 21C17, or an unlabeled isotype control, each at 10 μg/ml. After the incubation, the samples were stained with an immune profiling panel which included Abs against CD3, CD8, CD4, FOXP3, and two against CCR8: the commercially available anti-CCR8 Ab L263G8 (BioLegend) conjugated to phycoerythrin (PE), and mAb 21C17 conjugated to BRILLIANT VIOLET™ 421. After staining the cells, they were processed on a flow cytometer, and the data were analyzed and plotted using FLOWJO™ software (BD Biosciences, San Jose, CA).

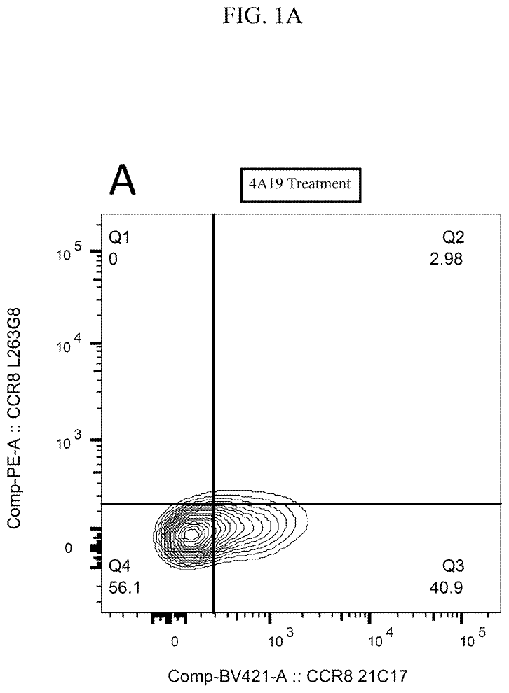

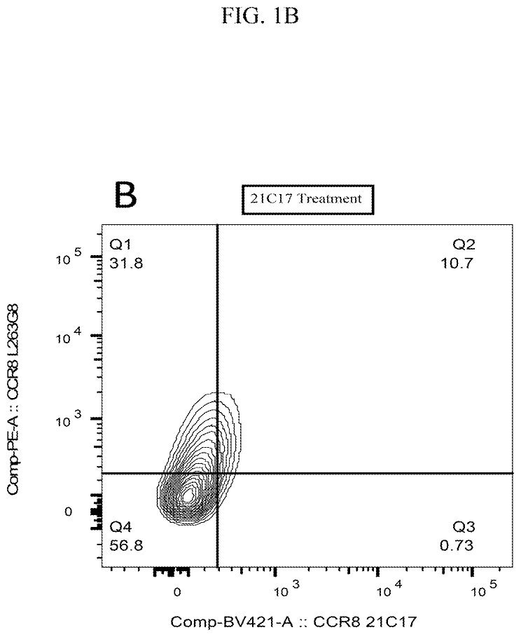

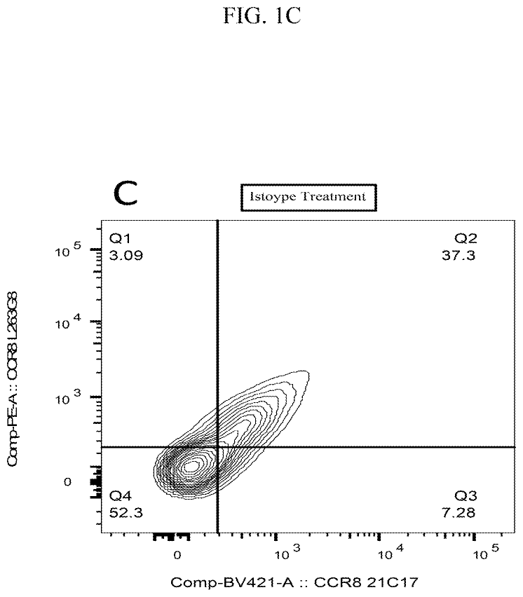

The three flow cytometric plots in FIG. 1 show the gastric tumor Treg compartment binding with BV421-conjugated mAb 21C17 on the x-axis and PE-conjugated Clone L263G8 on the y-axis. Tumor Tregs pretreated with 4A19 is shown in FIG. 1A. The results show that PE-L263G8 is blocked from binding to the 4A19-pretreated Tregs since no CCR8⁺ cells are seen in the Q1 or Q2 quadrants, indicating that binding of 4A19 to CCR8 blocks the subsequent binding of L263G8. In contrast, however, BV421-21C17 binds to these 4A19 pretreated cells because of the CCR8⁺ population seen in the Q3 quadrant (FIG. 1A). FIG. 1B, representing the unlabeled 21C17-treated sample, shows the opposite, which is that the BV421-21C17 Ab is blocked from binding (quadrants Q2 and Q3) whereas PE-L263G8 binds to the 21C17 pretreated Tregs since most of the CCR8⁺ population is in the Q1 quadrant. Thus, 21C17 does not block the binding of L263G8 but, as expected, blocks itself from binding to CCR8. FIG. 1C shows the effect of pretreatment with the isotype control, indicating that both 21C17 and PE-L263G8 are able to bind to the same cells since the most of the CCR8⁺ cells are in the Q2 quadrant. Overall, the results indicate that the 21C17 mAb binds to a different epitope on CCR8 than 4A19 since it is able to bind to tumor CCR8-expressing Tregs to which the 4A19 mAb had previously been bound. The lack of cross-competition between mAbs 4A9 and 21C17 for binding to hCCR8 suggests that they do not bind to substantially the same epitope region of hCCR8, i.e., their epitopes are not adjacent to or overlapping with each other such that binding of one Ab to its epitope sterically hinders the binding of the other Ab to its epitope.

### Example 4

Antibodies that Bind with High Affinity to an Epitope Outside the CCR8 N-Terminus
EC₅₀ Values for Binding of MAbs to Epitope Outside N-Terminus of hCCR8

FACS was used to evaluate the binding specificity of Abs binding to CCR8. Transfected Raji cells overexpressing hCCR8, and parental Raji cells that do not express CCR8, were incubated with viability dye on ice for 30 min. Unbound dye was washed away and cells were plated into 96-well round bottom plates at a concentration of 5×10⁵ cells per well in FACS staining buffer (PBS w/o Ca/Mg, 2 mM EDTA, 0.2% BSA). Serial dilutions of mAbs that bind to an epitope other than an N-terminal epitope of CCR8 were added, starting at a concentration of 20 nM, and incubated on ice for 30 min. Unbound Abs were washed off and a detection secondary Ab was added and incubated for 30 min on ice. Additional washes were performed and Ab binding affinities were determined by measuring the fluorescence intensities of the secondary Abs on a LSRFortessa X-20 cytometer (BD Biosciences).

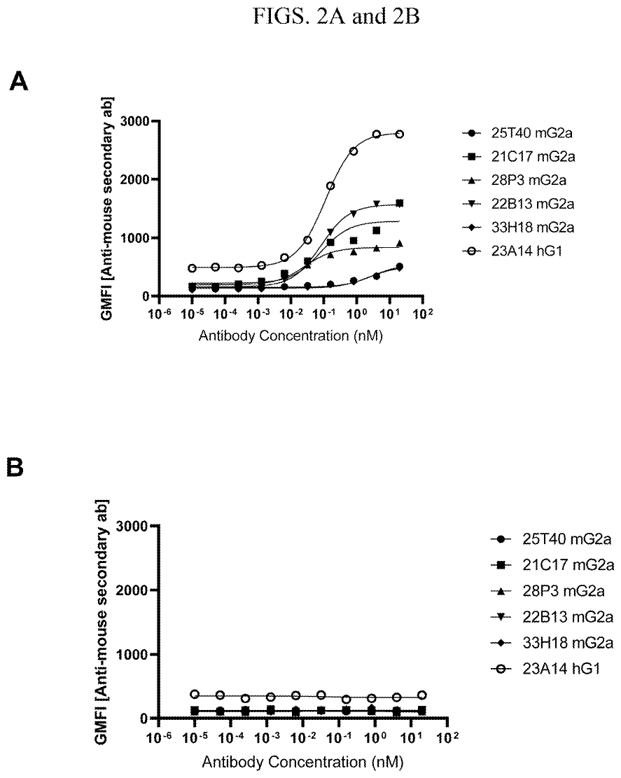

Binding curves showing the extent of binding of different concentrations of mAbs to hCCR8-expressing Raji cells are shown in FIG. 2A, whereas the absence of binding to the parental Raji cells is shown in FIG. 2B. This binding analysis identified 4 mAbs binding to Raji-hCCR8 cells with an EC₅₀ of about 0.1 nM or lower (see Table 1), with no nonspecific binding observed on parental Raji cells.

TABLE 1
CCR8 Ab binding on Raji-hCCR8

| Ab Clone | EC₅₀ (nM) Raji-hCCR8 Cells | EC₅₀ (nM) Raji Cells |
|---|---|---|
| 25T40mIgG2a | 3.015 | N/A |
| 21C17 mIgG2a | 0.08069 | N/A |
| 28P3 mIgG2a | 0.02686 | N/A |
| 22B13 mIgG2a | 0.08153 | N/A |
| 33H18 mIgG2a | 2.245 | N/A |
| 23A14hIgG1 | 0.1118 | N/A |

Competitive Binding Between N-Terminal-Binding Vs. Non-N-Terminal-Binding MAbs

Competition for binding to hCCR8 between an anti-CCR8 mAb that does not bind to the N-terminus of CCR8 (23A14-hIgG1) and an N-terminus-binding mAb (4A19-mIgG2a) was assayed by FACS. To isolate human Tregs (hTregs), PBMCs were isolated from a leukopak (AllCells) using a Ficoll density gradient (GE Healthcare). CD25⁺ cells were magnetically enriched using anti-CD25 MicroBeads II (Miltenyi Biotec). Enriched cells were stained for CD25 (4E3, Miltenyi Biotec), CD127 (A019D5, BioLegend), CD45RA (HI100, BioLegend), and CD4 (SK3, BD Biosciences). CD4⁺ CD127_{low} CD25_{high} CD45RA⁺ Tregs were sorted on a BD FACSAria II.

Isolated naïve Tregs were activated in vitro with DYNABEADS™ Human T-Activator CD3/CD28 beads at a 1:3 cell to bead ratio in the presence of 100 U/ml of rhIL-2 to stimulate expression of CCR8. Activated hTregs were incubated with viability dye for 30 min on ice. Unbound dyes were washed away and cells were plated into 96-well round bottom plates at a concentration of 5×10⁵ cells per well in FACS staining buffer. The mouse IgG2a original version of anti-CCR8 mAb 4A19 described in WO 2021/194942 (4A19-mIgG2a) was added to the cells at a saturating concentration of 200 nM and incubated on ice for 30 min. Unbound Abs were washed off and cells were incubated with mAb 23A14-hIgG1 serially titrated down from 200 nM to 0.0034 nM. Additional washes were performed and fluorophore-conjugated secondary Abs specific to human and mouse Ig's were added and incubated for 30 min. Final washes were preformed and samples were analyzed on a LSRFortessa X-20 cytometer.

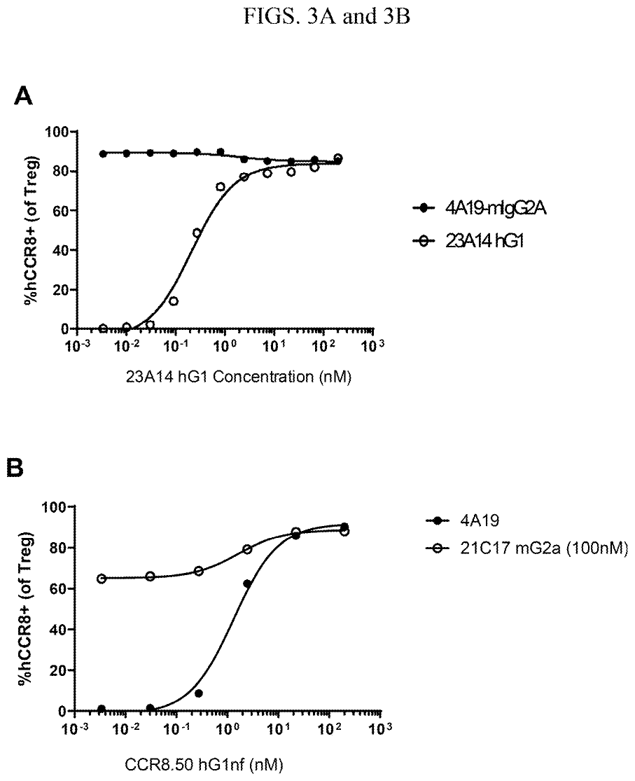

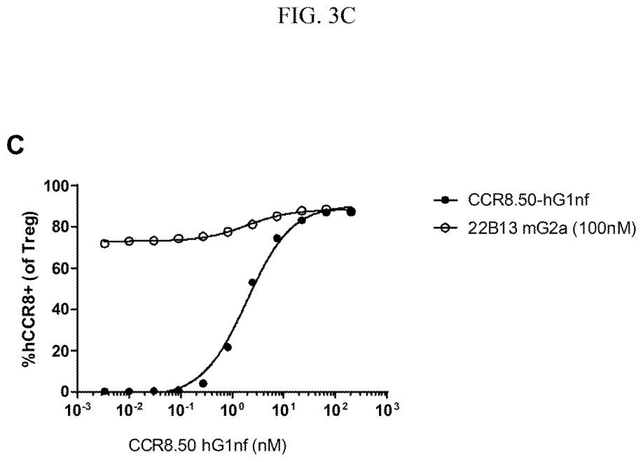

As shown in FIG. 3A, the binding of mAb 23A14-hIgG1 increases in the presence of saturating amounts of bound 4A19-mIgG2a, indicating that the binding of mAb 4A19-mIgG2a does not impede the binding of mAb 23A14-hIgG1. This competition assay analysis showed that mAbs 4A19-mIG2a and 23A14-hIgG1, tested at saturating concentrations, were able to detect equal population of CCR8⁺ Tregs.

Two additional anti-CCR8 mAbs that do not bind to an N-terminal epitope (21C17-mIgG2a and 22B13-mIgG2a) were tested by incubating activated hTregs stained with viability dye and serially titrated concentrations of mAb 4A19 (WO 2021/194942) ranging from 200 nM to 0.0034 nM. After a 30-min incubation on ice and washing, a fixed concentration (100 nM) of 21C17-mIgG2a or 22B13-mIgG2a was added and incubated on ice for 30 min. Cells were washed and detection Abs specific to human and mouse Ig's were added and incubated for 30 min on ice. Final wash steps were performed and samples were analyzed on a LSRFortessa X-20 cytometer.

The results obtained with 21C17-mIgG2a and 22B13-mIgG2a are shown in FIGS. 3B and C, respectively. Competition assay analysis shows that mAb 4A19, when paired with either 21C17-mIgG2a or 22B13-mIgG2a, is able to detect a similar population of CCR8⁺ human Tregs at saturating concentration.

### Example 5

Fluorochrome-Labeled Ab Binding to hCCR8-Expressing Cells

A flow cytometric assay was used to evaluate the binding of an anti-hCCR8 Ab on cell lines over expressing hCCR8. In this assay, Raji cells overexpressing hCCR8 were used to determine the binding of mAb 21C17-mIgG2a directly conjugated with BV-421 by BioLegend. Unlabeled 21C17-mIgG2a was used as a positive control. Binding to parental Raji cells that do not express CCR8 was also tested to assess the specificity of the mAbs. The parental Raji cells and Raji cells overexpressing hCCR8 were individually mixed with serial dilutions (20 to 0.0034 nM) of unlabeled 21C17-mIgG2a, BV-421-labeled 21C17-mIgG2a, or KLH-mIgG2a control mAbs. Binding of unlabeled 21C17-mIgG2a to cell surface hCCR8 was detected using a PE-labeled anti-mouse IgG (Fab′)2 fragment (Jackson ImmunoResearch). Relative cell binding was measured as the geometric mean fluorescence intensity (GMFI) of total cells positive for the PE-conjugated anti-mouse IgG Ab or the directly BV-421-labeled 21C17-mIgG2a Ab.

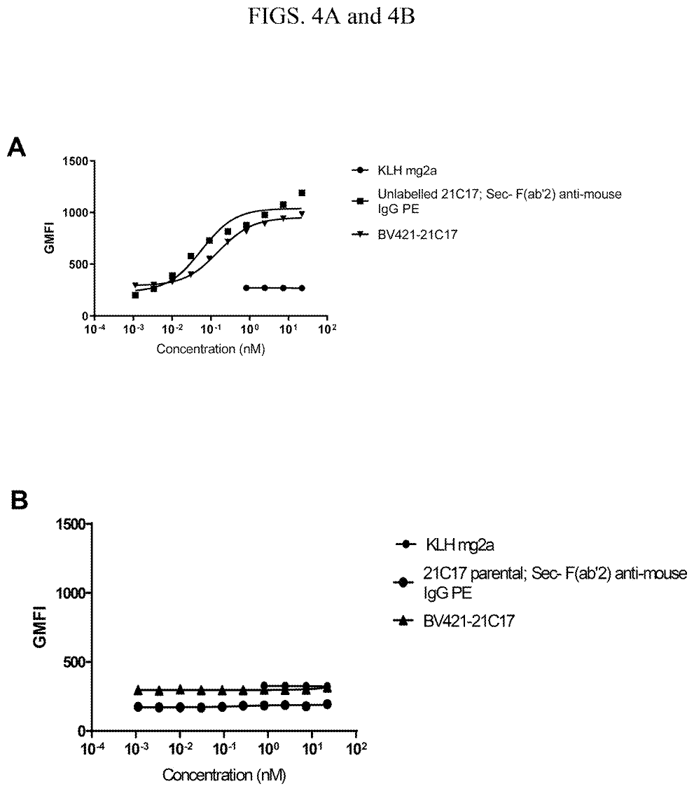

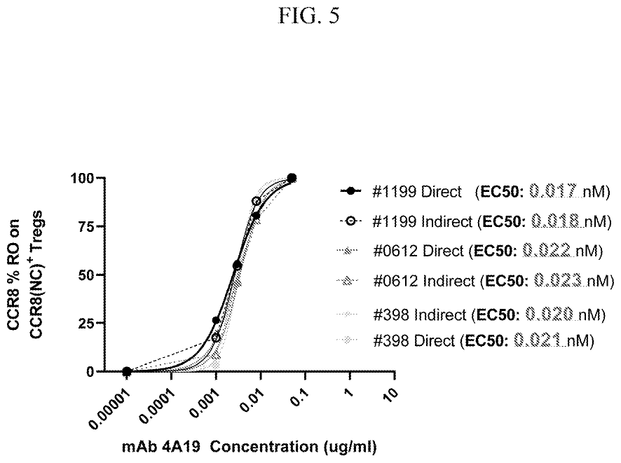

As shown in FIG. 4A, both the unlabeled and directly BV-421-labeled 21C17-mIgG2a Abs bind to the cell surface CCR8 on hCCR8-overexpressing Raji cell lines. These Abs were also confirmed to be binding specifically to hCCR8 as indicated by the absence of binding to parental Raji cells that do not express hCCR8 (FIG. 4B).

### Example 6

### Receptor Occupancy Assay for Assessing Target Engagement by Therapeutic Anti-CCR8 Antibody

Receptor occupancy (RO) assays are utilized in clinical trials for evaluating target engagement and determining dose selection, thereby providing valuable insights on pharmacodynamic and safety evaluation of a biotherapeutic. A flow cytometric assay using peripheral blood was developed and validated to measure CCR8 RO in a subject after administration of an anti-CCR8 therapeutic Ab.

Two independent RO assay formats were developed: (1) a direct/total format (measures bound and total receptor), and (2) an indirect/total format (measures free and total receptor). The latter represents a combination of an indirect RO and a total RO assay format; the indirect RO format employs a competing anti-CCR8 Ab to detect free CCR8 receptor unoccupied by the anti-CCR8 therapeutic Ab post-treatment to derive RO indirectly, whereas the total RO assay format utilizes a non-competing anti-CCR8 Ab to measure total CCR8 receptors available on Tregs for binding of the therapeutic anti-CCR8 Ab post-treatment. Both free and total CCR8 receptor information is used in deriving % RO in the indirect/total assay format using the formula described below.

Because the total number of CCR8 receptors on Tregs may fluctuate throughout the course of treatment with a therapeutic anti-CCR8 Ab, e.g., in a clinical study, use of a non-competing anti-CCR8 Ab is advantageous as it enables continuous monitoring of total CCR8 receptor levels, and allows for factoring in any changes in total receptor levels in response to anti-CCR8 Ab therapy when deriving % RO over the course of the treatment. Thus, the use of a non-competing anti-CCR8 Ab, i.e., an Ab that does not compete with the therapeutic Ab for binding to CCR8, is important for determining the modulation of total CCR8 expression levels and aiding in accurate % RO calculation.

Measurement of CCR8 RO is particularly challenging due to limited assay range as a result of low CCR8 expression on peripheral Tregs. However, by leveraging a non-competing anti-CCR8 Ab, a specific CCR8⁺ Treg subset was selected as the target cell population for the assay as opposed to the total Tregs. This served as an effective strategy for improving the assay dynamic range by 5×, thereby, making the indirect/total RO assay more robust and reliable.

A head-to-head comparison of the two distinct RO assay formats was performed. Whole blood samples (180 μl) were pre-treated with various concentrations of the therapeutic mAb, mAb 4A19 (WO 2021/194942), mimicking different 4A19 dose treatment levels in subjects in the dose escalation arm of a Phase I clinical study (NCT04895709), and pre-incubated for 1 h at 37° C. Aliquots of the treated samples were transferred to 4° C. for periods of 24, 48 and 72h for specimen stability experiments.

After pre-incubation, tubes containing background samples were treated with a saturating concentration (10 pg/ml) of mAb 4A19, while other tubes (containing free or bound or FMO [fluorescence minus one] samples) were treated with PBS (equal volume as mAb 4A19) for 30 min at room temperature (RT). Samples were centrifuged at 500×g for 5 min at 4° C., the pellets washed 3 times with GIBCO™'s Dulbecco's phosphate-buffered saline w/o Ca/Mg (DPBS; Thermo Fisher Scientific, Waltham, MA) and transferred into new tubes, then stained with a cocktail of 7 markers in the following core panel: Basic T cell makers: anti-human CD3 AF488 (BioLegend), anti-human CD4 BV510 (BioLegend) and anti-human CD8 BV605 (BD Biosciences); T cell differentiation marker: anti-human CD45RA APC-CY7 (BioLegend); and Treg markers: anti-human CD25 PE (BioLegend), anti-human CD127 APC-R700 (BD Biosciences), and anti-human CCR4 PE-Cy7 (BioLegend). One additional Ab was added to the core cocktail panel for the direct/total RO assay (custom conjugated anti-idiotypic Ab anti-Id AF647, Bristol Myers Squibb) for detecting bound CCR8 receptor, whereas two additional Abs were added to the cocktail panel for the indirect/total assay, a competing anti-hCCR8 Ab APC, clone 433H (BD Biosciences) for detecting free CCR8 receptors, and the non-competing anti-hCCR8 mAb, 21C17, for detecting total CCR8 receptors, for 30 min on ice. Once staining was completed, samples were washed twice with MACS buffer (Miltenyi Biotec) followed by red blood lysis with FACs lysing solution (proprietary buffered solution containing <10% formaldehyde and <50% diethylene glycol; BD Biosciences) for 15 min at RT in the dark. Post-lysing, samples were washed, then stored at 4° C. prior to sample acquisition on the same day on a flow cytometer. The % RO was derived for each 4A19 concentration point using the formula:

% RO=100×[1−((free postdose/free predose)/(total postdose/total predose))],

wherein the “free postdose” is the MFI determined by flow cytometry of the competing CCR8 Ab at each concentration of the therapeutic mAb (post exposure to the therapeutic mAb); “free predose” is the MFI of the competing CCR8 Ab when there is no exposure to the therapeutic mAb (pre exposure to the therapeutic mAb); “total postdose” is the MFI of the non-competing anti-CCR8 mAb at each concentration of the therapeutic mAb (post exposure to the therapeutic mAb); and “total predose” is the MFI of the non-competing anti-CCR8 mAb when there is no exposure to the therapeutic mAb (pre exposure to the therapeutic mAb).

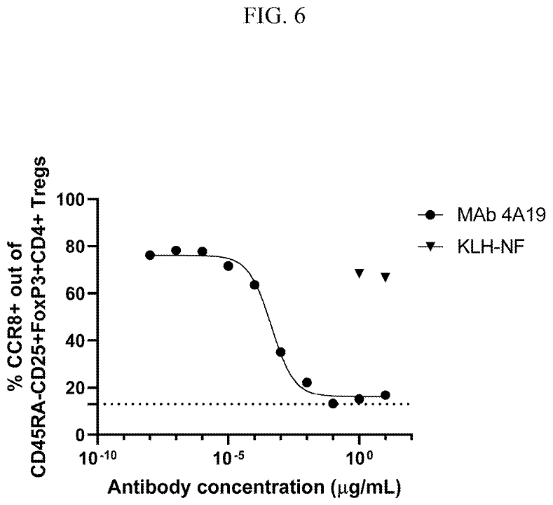

A response curve, i.e., the % RO vs. drug (e.g., mAb 4A19) concentration, was plotted, for example, as shown in FIG. 5. Both approaches yielded similar % RO drug dose-response curves and EC₅₀'s (e.g., for a particular donor, 0.022 nM vs. 0.023 nM, n=3), indicating the reliability of the developed assay. FIG. 5 is a representative figure demonstrating the change in CCR8% RO with increasing concentration of mAb 4A19 in healthy donor blood collected in sodium heparin (NaHep) and stored at 4° C. post-treatment for 24 h, n=3. The % RO curve was plotted using direct vs. indirect RO assay format for each donor. Both formats were independently validated for post-collection sample stability up to 72h, and acceptable intra- and inter-assay precision (Coefficient of Variation [CVs]≤30%). Additionally, consistent RO data were observed across the drug dose range from intra-subject longitudinally (CVs≤25%). The consistency of these data demonstrates that these CCR8 RO assays are sufficiently robust for use in clinical trials of a therapeutic anti-CCR8 Ab to evaluate and quantify engagement of the CCR8 target by the therapeutic Ab.

### Example 7

### Detection of CCR8 by Immunohistochemistry (IHC)

Single-stain IHC on formalin-fixed, paraffin-embedded (FFPE) specimens was used to detect and monitor CCR8 expression in tumor samples. IHC analysis was performed at room temperature using the Leica Bond Rx autostainer (Leica Biosystems, Buffalo Grove, IL). Specimens were sectioned at 4-μm thickness, mounted onto positive-charged glass slides, dried for at least 1 h with a fan, baked for 30 minutes at 60° C. in an oven, deparaffinized, and rehydrated offline. Tissues were then placed onto the autostainer, subjected to pretreatment using ER2 (Leica, Buffalo Grove, IL) for 30 min at 100° C., followed by rinsing and a 3-min incubation in Bond Wash Buffer (Leica). Tissues were incubated with Peroxide Block (Bond Polymer Refine Detection Kit, Leica), rinsed in Bond Wash Buffer (Leica), incubated with Human-to-Human protein block (Sigma; St. Louis, MO), followed by incubation with the primary Ab (mouse IgG2a,kappa clone 433H, BD Biosciences) or a mouse IgG1,kappa negative control antibody, for 30 min. Normal thymus tissue, which contains cellular features that are positive and negative for CCR8, was used as a positive and negative control.

Post incubation with the Ab, tissues were processed using the Bond Polymer Refine Detection Kit according to manufacturer's instructions, and incubated with 3,3′-diaminobenzidine (DAB) for 10 min followed by rinsing in distilled water. Tissues were incubated with hematoxylin for 5 min followed by a rinse in distilled water, a rinse in Bond Wash buffer, and a final rinse in distilled water.

Coverslip mounting was done using an automated glass coverslipper, and slides were scanned using an Aperio AT Turbo system (Aperio, Vista, CA) to produce whole slide photomicrographs. Images were analyzed by pathologist tumor cell score, by analysis with a CytoNuclear algorithm from Indica (Corrales, NM), and/or by pathologist visual immune score.

### Example 8

### Depletion of Tregs by MAb 4A19 in Dissociated Human Tumors

The ability of the therapeutic anti-CCR8 mAb, 4A19, to induce depletion of CCR8⁺ Tregs was assessed ex vivo in dissociated human tumors.

### Human Tumor Dissociation

Resected solid human tumors were shipped overnight at 4° C. in HypoThermosol biopreservation media (Charles River Laboratories, Wilmington, MA). Whole tumor samples were minced before dissociation with lab scissors. Tumors were then mechanically and enzymatically dissociated at 37° C. in Roswell Park Memorial Institute (RPMI)-1640 medium for 30 min using Miltenyi BioTec gentleMACS™ C Tubes, dissociator, and tumor dissociation kit. The resulting single cell suspensions were filtered through a 70-μm cell strainer, then processed for flow cytometry.

### Processing Cells for Flow Cytometry

After generating single cell suspensions from tumor tissues, cells were washed with PBS, then stained with amine-reactive viability dye for 20 min on ice. Cells were washed and blocked for 20 min on ice in blocking solution comprising 2% rat serum (MilliporeSigma, St. Louis, MO), 2% mouse serum (SouthernBiotech, Birmingham, AL), 10% human A/B serum (Gemini Bio, West Sacramento, CA), 1:250 dilution of human FcX (BioLegend), and 1:100 dilution of monocyte block (BioLegend), diluted in FACS staining buffer (PBS w/o Ca/Mg, 2 mM EDTA, 0.2% BSA). After blocking, cells were stained with UV-excitable dye-conjugated anti-human CD3, CD4, and CD8 mAbs (BD Biosciences) to define T cell subsets, whereas anti-human CD14 and CD56 mAbs (BD Biosciences) were used to define macrophages, monocytes, and NK cells. In addition to lineage markers, anti-human CCR8, CCR4, and CD16 Abs were used to identify CCR8⁺ tumor Tregs. Cells were then fixed and permeabilized with FOXP3 transcription factor staining buffer set for 30 min at 4° C., followed by another 30 min incubation at 4° C. with anti-FOXP3 Ab for intracellular staining. Finally, stained cells were washed and filtered using a 40-μm cell strainer.

Flow cytometric analyses were performed using a BD LSRFortessa™ X-20 flow cytometer, and population percentages and mean fluorescence intensity values were calculated using FLOWJO™ software analysis.

Depletion of Tregs by MAb 4A19 in Dissociated Human Tumors from Cancer Patients

Filtered single cells from the dissociated tumor tissues were collected and washed with complete RPMI-1640 media. Cells were resuspended in complete RPMI-1640 media and cultured with serial dilutions of mAb 4A19, or anti-KLH isotype control mAb for 48 h. Cells were seeded at a concentration of 500,000 cells per well in a flat-bottomed plate. After 48 h of incubation, cells were processed for flow cytometry analysis as described above. The percentage of depleted CCR8⁺ Tregs was determined by gating for CCR8⁺ cells within the parent gate FoxP3⁺, CD25^{high} and CD45RA⁻. The frequency of CCR8⁺ cells was calculated using FLOWJO™ software analysis, and depletion graphs were generated using PRISM software (GraphPad Software, San Diego, CA).

Treatment with mAb 4A19 resulted in depletion of CCR8⁺ Tregs in dissociated tumor tissues from cancer patient donors (n=5). The percentages of CCR8⁺ Treg depletion in these tumor tissues at the concentrations where maximum depletion was observed (1 μg/ml or 0.1 μg/ml) are summarized in Table 2. The percentage depletion of CCR8⁺ Tregs compared to KLH isotype control was calculated using the formula:

(1−(CCR8⁺ Tregs out of parent gate in A419-treated samples)/(CCR8⁺ Tregs out of parent gate in KLH isotype-treated samples)×100%.

As shown in FIG. 6, mAb 4A19 induces dose-dependent depletion of CCR8⁺ tumor Tregs whereas anti-KLH isotype Ab does not deplete Tregs even at the highest dose tested. This depletion plot is from a single patient and is representative of similar depletion assays from 5 patient donors. The parent gate represents a CD3⁺CD4⁺FoxP3⁺CD45RA population.

TABLE 2
Depletion of Tregs in Dissociated Human Tumors by MAb 4A19

| | Maximum Depletion (%) of CCR8⁺ Tregs |
|---|---|
| Tumor Types | Relative to KLH Isotype Control |
| Endometrial cancer | 69 |
| Renal cell carcinoma | 40 |
| Head and neck squamous | 25 |
| cell carcinoma | |
| Renal cell carcinoma | 51 |
| Renal cell carcinoma | 60 |

### EXEMPLARY EMBODIMENTS

The disclosed invention includes the following non-exhaustive listing of embodiments. This listing should not be construed to be in anyway limiting, and a person skilled in the art will appreciate that various modifications can be made to these embodiments without changing the essence and scope of the invention disclosed herein.

1. A monoclonal antibody, or an antigen-binding portion thereof, that binds specifically to human C-C Motif Chemokine Receptor 8 (hCCR8) expressed on the surface of a cell, wherein the antibody or antigen-binding portion thereof binds to an epitope that is not in the N-terminal domain of hCCR8 and does not cross-compete with an antibody that binds to an N-terminal epitope for binding to hCCR8.
2. The monoclonal antibody or antigen-binding portion thereof of embodiment 1, wherein the sequence of hCCR8 is set forth as SEQ ID NO: 1.
3. The monoclonal antibody or antigen-binding portion thereof of embodiment 1 or 2, wherein the N-terminal epitope comprises at least one amino acid within a peptide having the sequence V₁₂T₁₃D₁₄Y₁₅Y₁₆Y₁₇P₁₈D₁₉I₂₀F₂₁S₂₂ (SEQ ID NO: 73).
4. The monoclonal antibody or antigen-binding portion thereof of any one of the preceding embodiments, wherein the N-terminal epitope comprises at least 2, 3, 4, 5, 6, 7, 8, 9, 10 or all 11 amino acids within a peptide having the sequence V₁₂T₁₃D₁₄Y₁₅Y₁₆Y₁₇P₁₈D₁₉I₂₀F₂₁S₂₂ (SEQ ID NO: 73).
5. The monoclonal antibody or antigen-binding portion thereof of any one of the preceding embodiments, wherein the N-terminal epitope comprises the amino acids having the sequence V₁₂T₁₃D₁₄Y₁₅Y₁₆Y₁₇P₁₈D₁₉I₂₀F₂₁S₂₂ (SEQ ID NO: 73).
6. The monoclonal antibody or antigen-binding portion thereof of any one of the preceding embodiments, wherein the N-terminal epitope consists of the amino acids having the sequence V₁₂T₁₃D₁₄Y₁₅Y₁₆Y₁₇P₁₈D₁₉I₂₀F₂₁S₂₂ (SEQ ID NO: 73).
7. The monoclonal antibody or antigen-binding portion thereof of any one of embodiments 3-6, wherein amino acids Y₁₅ and/or Y₁₇ are sulfated.
8. The monoclonal antibody or antigen-binding portion thereof of any one of the preceding embodiments, whose binding to hCCR8 is not affected by the presence of an antibody bound to an N-terminal epitope comprising at least 1, 2, 3, 4, 5, 6, 7, 8, 9, 10 or all 11 amino acids within a peptide having the sequence V₁₂T₁₃D₁₄Y₁₅Y₁₆Y₁₇P₁₈D₁₉I₂₀F₂₁S₂₂ (SEQ ID NO: 73).
9. The monoclonal antibody or antigen-binding portion thereof of any one of the preceding embodiments, wherein the antibody that binds to the N-terminal epitope is an antibody comprising:
(a) a heavy chain variable region CDR1 comprising consecutively linked amino acids having the sequence set forth as SEQ ID NO: 53; a heavy chain variable region CDR2 comprising consecutively linked amino acids having the sequence set forth as SEQ ID NO: 54; a heavy chain variable region CDR3 comprising consecutively linked amino acids having the sequence set forth as SEQ ID NO: 55; a light chain variable region CDR1 comprising consecutively linked amino acids having the sequence set forth as SEQ ID NO: 56; a light chain variable region CDR2 comprising consecutively linked amino acids having the sequence set forth as SEQ ID NO: 57; and a light chain variable region CDR3 comprising consecutively linked amino acids having the sequence set forth as SEQ ID NO: 58;
(b) a V_{H} comprising consecutively linked amino acids having the sequence set forth as SEQ ID NO: 9 and a V_{L} comprising consecutively linked amino acids having the sequence set forth as SEQ ID NO: 16; and/or
(c) a heavy chain comprising consecutively linked amino acids having the sequence set forth as SEQ ID NO: 65 and a light chain comprising consecutively linked amino acids having the sequence set forth as SEQ ID NO: 72.
   10. The monoclonal antibody or antigen-binding portion thereof of any one of the preceding embodiments, which binds to a cell surface-expressed hCCR8 polypeptide in a formalin-fixed, paraffin-embedded (FFPE) tissue sample.
   11. The monoclonal antibody or antigen-binding portion thereof of any one of the preceding embodiments, which specifically binds to human CCR8-expressing Raji cells with an EC₅₀, as measured by the binding assay described in Example 4, of:
(a) about 50 nM or lower;
(b) about 3 nM or lower;
(c) about 0.5 nM or lower;
(d) about 0.1 nM or lower;
(e) about 0.01 nM or lower;
(f) about 0.005 nM or lower;
(g) about 0.1 nM;
(h) between about 0.005 nM and about 50 nM;
(i) between about 0.02 nM and about 3 nM; or
(j) between about 0.08 nM and about 2 nM.
   12. The monoclonal antibody or antigen-binding portion thereof of any one of the preceding embodiments, which cross-competes for binding to hCCR8 with a reference antibody, wherein the reference antibody comprises:
(a) a V_{H} comprising consecutively linked amino acids having the sequence set forth as SEQ ID NO: 4 and a V_{L} comprising consecutively linked amino acids having the sequence set forth as SEQ ID NO: 11;
(b) a V_{H} comprising consecutively linked amino acids having the sequence set forth as SEQ ID NO: 6 and a V_{L} comprising consecutively linked amino acids having the sequence set forth as SEQ ID NO: 13; or
(c) a V_{H} comprising consecutively linked amino acids having the sequence set forth as SEQ ID NO: 8 and a V_{L} comprising consecutively linked amino acids having the sequence set forth as SEQ ID NO: 15.
   13. The monoclonal antibody or antigen-binding portion thereof of any one of the preceding embodiments, which binds to the same epitope as does a reference antibody, wherein the reference antibody comprises:
(a) a V_{H} comprising consecutively linked amino acids having the sequence set forth as SEQ ID NO: 4 and a V_{L} comprising consecutively linked amino acids having the sequence set forth as SEQ ID NO: 11;
(b) a V_{H} comprising consecutively linked amino acids having the sequence set forth as SEQ ID NO: 6 and a V_{L} comprising consecutively linked amino acids having the sequence set forth as SEQ ID NO: 13; or
(c) a V_{H} comprising consecutively linked amino acids having the sequence set forth as SEQ ID NO: 8 and a V_{L} comprising consecutively linked amino acids having the sequence set forth as SEQ ID NO: 15.
   14. The monoclonal antibody or antigen-binding portion thereof of any one of the preceding embodiments, which comprises the following CDR domains as defined by the Kabat method:
(a) a heavy chain variable region CDR1 comprising consecutively linked amino acids having the sequence set forth as SEQ ID NO: 23; a heavy chain variable region CDR2 comprising consecutively linked amino acids having the sequence set forth as SEQ ID NO: 24; a heavy chain variable region CDR3 comprising consecutively linked amino acids having the sequence set forth as SEQ ID NO: 25; a light chain variable region CDR1 comprising consecutively linked amino acids having the sequence set forth as SEQ ID NO: 26; a light chain variable region CDR2 comprising consecutively linked amino acids having the sequence set forth as SEQ ID NO: 27; and a light chain variable region CDR3 comprising consecutively linked amino acids having the sequence set forth as SEQ ID NO: 28;
(b) a heavy chain variable region CDR1 comprising consecutively linked amino acids having the sequence set forth as SEQ ID NO: 35; a heavy chain variable region CDR2 comprising consecutively linked amino acids having the sequence set forth as SEQ ID NO: 36; a heavy chain variable region CDR3 comprising consecutively linked amino acids having the sequence set forth as SEQ ID NO: 37; a light chain variable region CDR1 comprising consecutively linked amino acids having the sequence set forth as SEQ ID NO: 38; a light chain variable region CDR2 comprising consecutively linked amino acids having the sequence set forth as SEQ ID NO: 39; and a light chain variable region CDR3 comprising consecutively linked amino acids having the sequence set forth as SEQ ID NO: 40; or
(c) a heavy chain variable region CDR1 comprising consecutively linked amino acids having the sequence set forth as SEQ ID NO: 47; a heavy chain variable region CDR2 comprising consecutively linked amino acids having the sequence set forth as SEQ ID NO: 48; a heavy chain variable region CDR3 comprising consecutively linked amino acids having the sequence set forth as SEQ ID NO: 49; a light chain variable region CDR1 comprising consecutively linked amino acids having the sequence set forth as SEQ ID NO: 50; a light chain variable region CDR2 comprising consecutively linked amino acids having the sequence set forth as SEQ ID NO: 51; and a light chain variable region CDR3 comprising consecutively linked amino acids having the sequence set forth as SEQ ID NO: 22.
   15. The monoclonal antibody or antigen-binding portion thereof of embodiment 14, which comprises:
(a) a V_{H} comprising consecutively linked amino acids having the sequence set forth as SEQ ID NO: 4 and a V_{L} comprising consecutively linked amino acids having the sequence set forth as SEQ ID NO: 11;
(b) a V_{H} comprising consecutively linked amino acids having the sequence set forth as SEQ ID NO: 6 and a V_{L} comprising consecutively linked amino acids having the sequence set forth as SEQ ID NO: 13; or
(c) a V_{H} comprising consecutively linked amino acids having the sequence set forth as SEQ ID NO: 8 and a V_{L} comprising consecutively linked amino acids having the sequence set forth as SEQ ID NO: 15.
   16. The monoclonal antibody or antigen-binding portion thereof of embodiment 14 or 15, which comprises:
(a) a heavy chain comprising consecutively linked amino acids having the sequence set forth as SEQ ID NO: 60 and a light chain comprising consecutively linked amino acids having the sequence set forth as SEQ ID NO: 67;
(b) a heavy chain comprising consecutively linked amino acids having the sequence set forth as SEQ ID NO: 62 and a light chain comprising consecutively linked amino acids having the sequence set forth as SEQ ID NO: 69; or
(c) a heavy chain comprising consecutively linked amino acids having the sequence set forth as SEQ ID NO: 64 and a light chain comprising consecutively linked amino acids having the sequence set forth as SEQ ID NO: 71.
   17. The monoclonal antibody or antigen-binding portion thereof of any one of embodiments 14-16, which is the monoclonal antibody designated herein 21C17, 22B13, or 23A14, or an antigen-binding portion thereof.
   18. The monoclonal antibody or antigen-binding portion thereof of any one of the preceding embodiments, which is a chimeric antibody, a humanized antibody, a human antibody, or a fragment thereof.
   19. The monoclonal antibody or antigen-binding portion thereof of any one of embodiments 1-15, which comprises a heavy chain constant region which is of a human IgG1, IgG2, IgG3 or IgG4 isotype.
   20. The monoclonal antibody or antigen-binding portion thereof of any one of embodiments 1-15, which comprises a heavy chain constant region which is of a human IgG2 or IgG4 isotype.
   21. A labeled antibody, or an antigen-binding thereof, comprising the monoclonal antibody or antigen-binding thereof of any one of the preceding embodiments and a detectable label.
   22. The labeled antibody or antigen-binding thereof of embodiment 21, wherein the detectable label is a fluorophore, a chromophore, an enzyme, a radioactive isotope, a micropolymer, or a metal.
   23. The labeled antibody of embodiment 21, wherein the detectable label is biotin.
   24. The labeled antibody of embodiment 22, wherein the fluorophore is a Brilliant Violet™ dye (e.g., BV-421), an AmCyan dye, an Alexa Fluor® dye, a Cy® dye, a CF® dye, fluorescein isothiocyanate (FITC), tetramethylrhodamine (TRITC) phycoerythrin (PE), allophycocyanin (APC), or peridinin-chlorophyll protein (PerCP).
   25. The labeled antibody of embodiment 22, wherein the chromophore is a porphyrin, pyropheophorbide-α, benzoporphyrin monoacid ring A (BPDMA), or chlorin e6.
   26. The labeled antibody of embodiment 22, wherein the enzyme is alkaline phosphatase, horseradish peroxidase, glucose oxidase or β-galactosidase.
   27. The labeled antibody of embodiment 22, wherein the radioactive isotope is ⁸⁹Zr, ⁶⁴Cu, ⁸⁶Y, ¹¹C, ¹⁸F, ⁶⁸Ga, ⁵²Mn, ⁵⁵Co, ¹⁵²Tb, ⁹⁰Nb, ⁶⁶Ga, ⁷²As, I¹²⁵, or ⁶⁹Ge.
   28. The labeled antibody of embodiment 22, wherein the metal label is yttrium (Y), indium (In), the series of lanthanide elements (Ln, from La to Lu, except Pm), iodine (I), cadmium (Cd), tellurium (Te), silver (Ag), palladium (Pd), rhodium (Rh), iridium (Ir), platinum (Pt), ruthenium (Ru), osmium (Os) or bismuth (Bi).
   29. An immunoconjugate comprising the monoclonal antibody or antigen-binding portion thereof of any one of embodiments 1-20, linked to a cytolytic agent.
   30. The immunoconjugate of embodiment 28, wherein the cytolytic agent is a cytotoxin, a radioactive isotope, or a photosensitizer.
   31. A chimeric antigen receptor (CAR) comprising the monoclonal antibody or antigen-binding portion thereof of any one of embodiments 1-20.
   32. A T cell receptor (TCR) comprising the monoclonal antibody or antigen-binding portion thereof of any one of embodiments 1-20.
   33. A bispecific molecule comprising the monoclonal antibody or antigen-binding portion thereof of any one of embodiments 1-20, linked to a binding domain that has a different binding specificity than the monoclonal antibody or antigen binding portion thereof.
   34. A composition comprising:
(a) the monoclonal antibody or antigen-binding portion thereof of any one of embodiments 1-20;
(b) the labeled antibody or antigen-binding portion thereof of any one of embodiments 21-28;
(c) the immunoconjugate of embodiment 29 or 30;
(d) the CAR of embodiment 31,
(e) the TCR of embodiment 32, or
(f) the bispecific molecule of embodiment 33,
and a pharmaceutically acceptable carrier.
   35. An isolated nucleic acid encoding the monoclonal antibody or antigen-binding portion thereof of any one of embodiments 1-20.
   36. An expression vector comprising the nucleic acid of embodiment 35.
   37. A host cell comprising the expression vector of embodiment 36.
   38. A method for preparing an anti-CCR8 antibody or antigen-binding portion thereof which comprises expressing the antibody or antigen-binding portion thereof in the host cell of embodiment 37 and isolating the antibody or antigen-binding portion thereof from the host cell.
   39. A method for generating a first antibody that does not bind, or does not cross-compete with a second antibody for binding, to a defined epitope on an antigen, which comprises immunizing a vertebrate with an immunogen comprising a cell line, or a component of said cell line, that expresses the antigen and also expresses a second antibody or antigen-binding portion thereof that binds specifically to the epitope, wherein the binding of the second antibody or antigen-binding portion thereof to the epitope shields said epitope from the immune system of the vertebrate, reducing the generation of antibodies that bind to the epitope and thereby preferentially resulting in the generation of a first antibody that does not bind, or does not cross-compete with the second antibody for binding, to the epitope.
   40. The method of embodiment 39, wherein the antigen comprises a CCR8 receptor.
   41. The method of embodiment 40, wherein the CCR8 receptor is a hCCR8 receptor.
   42. The method of any one of embodiments 39-41, wherein the vertebrate is a mammal or a bird.
   43. The method of any embodiment 42, wherein the mammal is a mouse, rat, hamster, rabbit, dog, goat, sheep or horse.
   44. The method of any one of embodiments 39-43, wherein the immunogen is a detergent-stabilized proteoliposome component of the cell line.
   45. The method of embodiment 41, wherein the epitope is an epitope in the N-terminal domain of the hCCR8 receptor.
   46. The method of embodiment 44, wherein the second antibody or antigen-binding portion thereof is the monoclonal antibody designated L263G8 (BioLegend), the monoclonal antibody designated 433H (BD Biosciences), 4A19, 18Y12, 10R3, 8D55, 14S15, or 14S15h.
   47. A method for measuring receptor occupancy (RO) of a cell membrane-bound CCR8 receptor to which a therapeutic Treg-depleting anti-CCR8 antibody binds comprising:
(a) adding to background whole blood samples, previously exposed to various concentrations of the therapeutic antibody, a saturating concentration of the therapeutic antibody;
(b) incubating aliquots of free, bound or fluorescence minus one (FMO) samples with buffer not containing the therapeutic antibody;
(c) staining the samples with a core panel of antibodies to identify basic T cell markers, T cell differentiation markers, and Treg markers;
(d) adding to the core panel
   (i) for the direct/total RO assay, an anti-idiotypic antibody for detecting bound CCR8 receptor, or
   (ii) for the indirect/total assay, two additional antibodies comprising an allophycocyanin (APC)-conjugated anti-hCCR8 antibody that competes with the therapeutic antibody, for detecting free CCR8 receptors, and an anti-hCCR8 antibody that does not compete with the therapeutic antibody, for detecting total CCR8 receptors;
(e) lysing the red blood cells to clear the samples and analyzing the cleared samples by flow cytometry; and
(f) determining the % RO for each concentration of the therapeutic antibody using the formula:

% RO=100×[1−((free postdose/free predose)/(total postdose/total predose))].

48. A method for measuring a depletion in the frequency of CCR8-expressing cells in the peripheral blood of a subject undergoing treatment with an anti-CCR8 mAb, the method comprising:
(a) determining a baseline level of expression of CCR8, and/or the frequency of CCR8-expressing cells in a first sample of whole blood or PBMCs from the subject;
(b) administering a treatment of an anti-CCR8 mAb to the subject; and
(c) determining the level of expression of CCR8, and/or the frequency of CCR8-expressing cells in a second sample of whole blood or PBMCs from the subject taken after administration of the anti-CCR8 mAb;
wherein a decrease in the level of expression of CCR8, and/or the frequency of CCR8-expressing cells in the second sample indicates that the number of CCR8-expressing cells in the blood has been depleted.
   49. A method for measuring a depletion in the number of tumor-infiltrating CCR8-expressing Tregs in a subject undergoing treatment, the method comprising:
(a) determining a baseline level of expression of CCR8, and/or the frequency of CCR8-expressing Tregs, in a first sample of test tissue in or from the subject, the test tissue comprising tumor cells and tumor-infiltrating Tregs;
(b) administering a treatment of an anti-CCR8 antibody to the subject; and
(c) determining the level of expression of CCR8, and/or the frequency of CCR8-expressing Tregs, in a second sample of test tissue in or from a subject, the second test tissue taken during or after the treatment;
wherein a decrease in the level of expression of CCR8, and/or a decrease in the frequency of CCR8-expressing Tregs, in the second sample indicates that the number of Tregs in the test tissue has been depleted.
   50. The method of embodiment 49, wherein a depletion in the number of Tregs in the test tissue indicates an enhancement in an immune response in the subject.
   51. The method of embodiment 49 or 50, wherein the treatment administered to the subject is a treatment for cancer.
   52. The method of embodiment 51, wherein the treatment for cancer comprises administration of a therapeutic Treg-depleting antibody or an antigen-binding portion thereof.
   53. The method of embodiment 52, wherein the therapeutic Treg-depleting antibody or antigen-binding portion thereof is an anti-CCR8 antibody or antigen-binding portion thereof.
   54. A method for measuring a depletion in the number of skin T cells in a subject undergoing treatment, the method comprising:
(a) determining a baseline level of expression of CCR8 in a first sample of test tissue in or taken from the subject, the test tissue comprising skin T cells;
(b) administering a treatment of an anti-CCR8 antibody to the subject; and
(c) determining the level of expression of CCR8 in a second test tissue in or from the subject, the second sample of test tissue taken during or after the treatment;
wherein a decrease in the level of expression of CCR8 in the second test tissue indicates that the number of skin T cells in the test tissue has been depleted.
   55. A method for predicting the effectiveness of a therapeutic Treg-depleting antibody or an antigen-binding portion thereof in treating cancer in a subject, which method comprises:
(a) determining the level of expression of CCR8, and/or the frequency of CCR8-expressing Tregs, in a test tissue in or from the subject, the test tissue comprising tumor cells and tumor-infiltrating Tregs;
(b) comparing the level of expression of CCR8, and/or the frequency of CCR8-expressing Tregs, with a predetermined threshold value; and
(c) predicting the effectiveness of the therapeutic Treg-depleting antibody or antigen-binding portion thereof,
wherein a level of expression of CCR8, and/or the frequency of CCR8-expressing Tregs, exceeding the threshold value indicates that the therapeutic antibody or antigen-binding portion thereof will be effective in treating the subject, and wherein a level of expression of CCR8, and/or the frequency of CCR8-expressing Tregs, less than the threshold value indicates that the therapeutic antibody or antigen-binding portion thereof will not be effective in treating the subject.
   56. A method for predicting the effectiveness of a therapeutic Treg-depleting antibody or an antigen-binding portion thereof in treating cancer in a subject, which method comprises:
(a) determining the level of surface expression of CCR8, and/or the frequency of CCR8-expressing Tregs, in a test tissue in or taken from the subject, the test tissue comprising tumor cells and tumor-infiltrating Tregs;
(b) administering the therapeutic Treg-depleting antibody or antigen-binding portion thereof the subject;
(c) determining whether there is a decrease in the frequency of CCR8-expressing Tregs after administration of the therapeutic Treg-depleting antibody or antigen-binding portion thereof, and
(d) predicting that the therapeutic Treg-depleting antibody or antigen-binding portion thereof will be effective in treating cancer in the subject if the decrease in the frequency of CCR8-expressing Tregs exceeds a predetermined threshold value, or
(e) predicting that the therapeutic Treg-depleting antibody or antigen-binding portion thereof will not be effective in treating cancer in the subject if the decrease in the frequency of CCR8-expressing Tregs is less than a predetermined threshold value.
   57. A method for selecting a subject afflicted with a cancer as a suitable candidate for cancer immunotherapy with a therapeutic Treg-depleting antibody or an antigen-binding portion thereof, which method comprises:
(a) determining the level of expression of CCR8, and/or the frequency of CCR8-expressing Tregs, in a test tissue in or from the subject, the test tissue comprising tumor cells and tumor-infiltrating Tregs;
(b) comparing the level of expression of CCR8, and/or the frequency of CCR8-expressing Tregs, with a predetermined threshold value; and
(c) selecting the subject as a suitable candidate for immunotherapy with the therapeutic Treg-depleting antibody or antigen-binding portion thereof based on an assessment that the level of surface expression of CCR8, and/or the frequency of CCR8-expressing Tregs, in cells of the test tissue exceeds the predetermined threshold value.
   58. A method for selecting a subject afflicted with a cancer as a suitable candidate for cancer immunotherapy with a therapeutic Treg-depleting antibody or an antigen-binding portion thereof, which method comprises:
(a) determining the level of surface expression of CCR8, and/or the frequency of CCR8-expressing Tregs, in a test tissue in or taken from the subject, the test tissue comprising tumor cells and tumor-infiltrating Tregs;
(b) administering the therapeutic Treg-depleting antibody or antigen-binding portion thereof the subject;
(c) determining whether there is a decrease in the frequency of CCR8-expressing Tregs after administration of the therapeutic Treg-depleting antibody or antigen-binding portion thereof, and
(d) selecting the subject as a suitable candidate for immunotherapy with the therapeutic Treg-depleting antibody or antigen-binding portion thereof based on an assessment that a decrease in the frequency of CCR8-expressing Tregs exceeds a predetermined threshold value.
   59. A method for treating cancer in a subject, which method comprises:
(a) selecting a subject that is a suitable candidate for cancer immunotherapy with a therapeutic Treg-depleting antibody or an antigen-binding portion thereof, the selecting comprising:
   (i) determining the level of expression of CCR8, and/or the frequency of CCR8-expressing Tregs, in a test tissue in or from the subject, the test tissue comprising tumor cells and tumor-infiltrating Tregs;
   (ii) comparing the level of expression of CCR8, and/or the frequency of CCR8-expressing Tregs, with a predetermined threshold value; and
   (iii) selecting the subject as a suitable candidate for cancer immunotherapy with the therapeutic Treg-depleting antibody or antigen-binding portion thereof based on an assessment that the level of CCR8 expression in cells of the test tissue, and/or the frequency of CCR8-expressing Tregs, exceeds the predetermined threshold value; and
(b) administering to the selected subject a composition comprising a therapeutically effective amount of the therapeutic Treg-depleting antibody or antigen-binding portion thereof.
   60. A method for treating a subject afflicted with a cancer, which method comprises:
(a) selecting a subject that is a suitable candidate for cancer immunotherapy with a therapeutic Treg-depleting antibody or an antigen-binding portion thereof, the selecting comprising:
   (i) determining the level of surface expression of CCR8, and/or the frequency of CCR8-expressing Tregs, in a test tissue in or taken from the subject, the test tissue comprising tumor cells and tumor-infiltrating Tregs;
   (ii) administering the therapeutic Treg-depleting antibody or antigen-binding portion thereof to the subject;
   (iii) determining whether there is a decrease in the frequency of CCR8-expressing Tregs after administration of the therapeutic Treg-depleting antibody or antigen-binding portion thereof, and
   (iv) selecting the subject as a suitable candidate for immunotherapy with the therapeutic based on an assessment that a decrease in the frequency of CCR8-expressing Tregs exceeds a predetermined threshold value; and
(b) administering to the selected subject a composition comprising a therapeutically effective amount of the therapeutic Treg-depleting antibody or antigen-binding portion thereof.
   61. A method for treating a cancer in a subject, which method comprises:
(a) selecting a subject that is not a suitable candidate for cancer immunotherapy with a therapeutic Treg-depleting antibody or an antigen-binding portion thereof, the selecting comprising:
   (i) determining the level of expression of CCR8, and/or the frequency of CCR8-expressing Tregs, in a test tissue in or from the subject, the test tissue comprising tumor cells and tumor-infiltrating Tregs;
   (ii) comparing the level of expression of CCR8, and/or the frequency of CCR8-expressing Tregs, with a predetermined threshold value; and
   (iii) selecting the subject as not suitable for immunotherapy with the therapeutic Treg-depleting antibody or antigen-binding portion thereof based on an assessment that the level of CCR8 expression, and/or the frequency of CCR8-expressing Tregs, in cells of the test tissue is less than the predetermined threshold value; and
(b) administering a standard-of-care therapeutic other than the therapeutic Treg-depleting antibody or antigen-binding portion thereof to the selected subject.
   62. A method for treating a cancer in a subject, which method comprises:
(a) selecting a subject that is not a suitable candidate for cancer immunotherapy with a therapeutic Treg-depleting antibody or an antigen-binding portion thereof, the selecting comprising:
   (i) determining the level of surface expression of CCR8, and/or the frequency of CCR8-expressing Tregs, in a test tissue in or taken from the subject, the test tissue comprising tumor cells and tumor-infiltrating Tregs;
   (ii) administering the therapeutic Treg-depleting antibody or antigen-binding portion thereof the subject;
   (iii) determining whether there is a decrease in the frequency of CCR8-expressing Tregs after administration of the therapeutic Treg-depleting antibody or antigen-binding portion thereof, and
   (iv) selecting the subject as not suitable for immunotherapy with the therapeutic Treg-depleting antibody or antigen-binding portion thereof based on an assessment that the decrease in the frequency of CCR8-expressing Tregs is less than a predetermined threshold value; and
(b) administering to the selected subject a standard-of-care therapeutic other than the therapeutic anti-CCR8 antibody or antigen-binding portion thereof.
   63. A method for treating a cancer in a subject, which method comprises administering to the subject a composition comprising a therapeutically effective amount of a therapeutic Treg-depleting antibody or an antigen-binding portion thereof, the subject having been selected on the basis that the level of expression and/or the frequency of CCR8-expressing Tregs in a test tissue in or from the subject, the test tissue comprising tumor cells and tumor-infiltrating Tregs, was determined to exceed a predetermined threshold level.
   64. A method for treating cancer in a subject, which method comprises administering to the subject a composition comprising a therapeutically effective amount of a therapeutic Treg-depleting antibody or an antigen-binding portion thereof, the subject having been selected on the basis that the therapeutic Treg-depleting antibody or antigen-binding portion thereof causes a decrease in the frequency of CCR8-expressing Tregs that exceeds a predetermined threshold value.
   65. A method for treating a cancer in a subject, which method comprises administering to the subject a standard-of-care treatment other than a therapeutic Treg-depleting antibody or an antigen-binding portion thereof, the subject having been selected on the basis that the level of CCR8 in cells of, and/or the frequency of CCR8-expressing Tregs in, a test tissue in or from the subject, the test tissue comprising tumor cells and tumor-infiltrating Tregs, was determined to be less than a predetermined threshold level.
   66. A method for treating cancer in a subject, which method comprises administering to the subject a standard-of-care treatment other than a therapeutic Treg-depleting antibody or an antigen-binding portion thereof, the subject having been selected on the basis that the therapeutic Treg-depleting Ab or antigen-binding portion thereof causes a decrease in the frequency of CCR8-expressing Tregs that is less than a predetermined threshold level.
   67. The method of any one of embodiments 55-66, wherein the Treg-depleting antibody is an anti-CCR8, anti-CTLA-4, anti-CCR4 or anti-CD25 antibody.
   68. The method of any one of embodiments 48-67, wherein determining the level of expression of CCR8 in the test tissue comprises assessing the level of expression of CCR8 on the surface of Tregs in the test tissue.
   69. The method of any one of embodiments 48-67, wherein determining the level of expression of CCR8 in the test tissue comprises assessing the proportion of Tregs in the test tissue that express CCR8 on the Treg cell surface.
   70. The method of any one of embodiments 48-69, wherein the level of expression of CCR8 in the test tissue in the subject is determined by an in vivo method.
   71. The method of embodiment 70, wherein the in vivo method comprises PET tracing using the anti-CCR8 antibody of embodiment 1.
   72. The method of any one of embodiments 48-70, wherein the level of expression of CCR8 is determined ex vivo in a test tissue sample obtained from the subject.
   73. The method of embodiment 72, wherein the level of expression of CCR8 is determined by immunohistochemistry (IHC), flow cytometry, or mass spectrometry-coupled flow cytometry, using the labeled anti-CCR8 antibody or antigen-binding portion thereof of any one of embodiments 21-28 to bind to CCR8 expressed on the surface of a cell in the tissue.
   74. The method of embodiment 73, wherein the IHC is performed on fresh frozen tissue or formalin-fixed paraffin-embedded (FFPE) tissue.
   75. The method of embodiment 70 or 73, wherein determining the level of expression of CCR8 using the anti-CCR8 antibody of embodiment 1 is not affected by the binding of a therapeutic antibody or an antigen-binding portion thereof to the N-terminal domain of CCR8.
   76. The method of any one of embodiments 52-66 and 75, wherein the therapeutic anti-CCR8 antibody or antigen-binding portion thereof comprises:
(a) a heavy chain variable region CDR1 comprising consecutively linked amino acids having the sequence set forth as SEQ ID NO: 53; a heavy chain variable region CDR2 comprising consecutively linked amino acids having the sequence set forth as SEQ ID NO: 54; a heavy chain variable region CDR3 comprising consecutively linked amino acids having the sequence set forth as SEQ ID NO: 55; a light chain variable region CDR1 comprising consecutively linked amino acids having the sequence set forth as SEQ ID NO: 56; a light chain variable region CDR2 comprising consecutively linked amino acids having the sequence set forth as SEQ ID NO: 57; and a light chain variable region CDR3 comprising consecutively linked amino acids having the sequence set forth as SEQ ID NO: 58;
(b) a V_{H} comprising consecutively linked amino acids having the sequence set forth as SEQ ID NO: 9 and a V_{L} comprising consecutively linked amino acids having the sequence set forth as SEQ ID NO: 16; or
(c) a heavy chain comprising consecutively linked amino acids having the sequence set forth as SEQ ID NO: 65 and a light chain comprising consecutively linked amino acids having the sequence set forth as SEQ ID NO: 72.
   77. The method of any one of embodiments 52-66 and 75, wherein the therapeutic anti-CCR8 antibody or an antigen-binding portion thereof is the antibody designated 4A19, 18Y12, 8D55, 10R3, 14S15, or 14S15h, or an antigen-binding portion thereof.
   78. The method of any one of embodiments 49-77, wherein the subject is a human.
   79. The method of any one of embodiments 51-78, wherein the cancer is a solid tumor.
   80. The method of embodiment 79, wherein the solid tumor is a cancer chosen from squamous cell carcinoma, small cell lung cancer (SCLC), non-small cell lung cancer (NSCLC), squamous NSCLC, non-squamous NSCLC, head and neck cancer, breast cancer, cancer of the esophagus, gastric cancer, gastrointestinal cancer, cancer of the small intestine, liver cancer, hepatocellular carcinoma (HCC), pancreatic cancer (PAC), kidney cancer, renal cell carcinoma (RCC), bladder cancer, cancer of the urethra, cancer of the ureter, colorectal cancer (CRC), colon cancer, colon carcinoma, cancer of the anal region, endometrial cancer, prostate cancer, a fibrosarcoma, neuroblastoma, glioma, glioblastoma, germ cell tumor, pediatric sarcoma, sinonasal natural killer, melanoma, skin cancer, bone cancer, cervical cancer, uterine cancer, carcinoma of the endometrium, carcinoma of the fallopian tubes, ovarian cancer, carcinoma of the cervix, carcinoma of the vagina, carcinoma of the vulva, testicular cancer, cancer of the endocrine system, thyroid cancer, cancer of the parathyroid gland, cancer of the adrenal gland, sarcoma of soft tissue, cancer of the penis, carcinoma of the renal pelvis, neoplasm of the central nervous system (CNS), primary CNS lymphoma, tumor angiogenesis, spinal axis tumor, brain cancer, brain stem glioma, pituitary adenoma, Kaposi's sarcoma, epidermoid cancer, squamous cell cancer, solid tumors of childhood, environmentally-induced cancers, virus-related cancers, cancers of viral origin, advanced cancer, unresectable cancer, metastatic cancer, refractory cancer, recurrent cancer, and any combination thereof.
   81. The method of embodiment 79, wherein the solid tumor is a cancer chosen from head and neck squamous cell carcinoma (HNSC), lung adenocarcinoma (LUAD), stomach adenocarcinoma (STAD), lung squamous cell carcinoma (LUSC), pancreatic adenocarcinoma (PAAD), rectum adenocarcinoma (READ), esophageal carcinoma (ESCA), breast invasive carcinoma (BRCA), colon adenocarcinoma (COAD) and cervical squamous cell carcinoma and endocervical adenocarcinoma (CESC).
   82. The method of embodiment 79, wherein the solid tumor is a cancer chosen from colon adenocarcinoma, bladder carcinoma, mammary carcinoma, and fibrosarcoma.
   83. The method of embodiment 79, wherein the solid tumor is a cancer chosen from NSCLC, SCCHN, Microsatellite Stable colorectal cancer (MSS-CRC), gastric/gastroesophageal (GE) junction adenocarcinoma, and cervical cancer.
   84. The method of any one of embodiments 51-78, wherein the cancer is a hematological malignancy.
   85. The method of embodiment 84, wherein the hematological malignancy is selected from follicular lymphoma (FL) and acute lymphocytic leukemia and lymphoma.
   86. The method of embodiment 84, wherein the hematological malignancy is selected from acute lymphoblastic leukemia (ALL), acute myelogenous leukemia (AML), chronic lymphocytic leukemia (CLL), chronic myelogenous leukemia (CML), a T cell lymphoma, Hodgkin's lymphoma (HL), non-Hodgkin's lymphomas (NHLs), multiple myeloma, smoldering myeloma, monoclonal gammopathy of undetermined significance (MGUS), and any combinations of said hematological malignancies.
   87. The method of embodiment 84, wherein the hematological malignancy is selected from diffuse large B-cell lymphoma (DLBCL), CLL/small lymphocytic lymphoma (SLL), mantle cell lymphoma, mucosa-associated lymphoid tissue (MALT) lymphoma, nodal marginal zone B-cell lymphoma, splenic marginal zone B-cell lymphoma, Burkitt lymphoma, lymphoplasmacytoid lymphoma (LPL), hairy cell lymphoma, primary central nervous system (CNS) lymphoma, precursor T-lymphoblastic lymphoma/leukemia, T-lymphoblastic lymphoma/leukemia (T-Lbly/T-ALL), cutaneous T-cell lymphoma, adult T-cell lymphoma/leukemia, angioimmunoblastic T-cell lymphoma, extranodal natural killer/T-cell lymphoma nasal type, enteropathy-associated intestinal T-cell lymphoma (EATL), anaplastic large-cell lymphoma (ALCL), peripheral T-cell lymphoma unspecified, lymphoplasmacytoid lymphoma, monocytoid B cell lymphoma, angiocentric lymphoma, intestinal T-cell lymphoma, primary mediastinal B-cell lymphoma, post-transplantation lymphoproliferative disorder, true histiocytic lymphoma, primary effusion lymphoma, diffuse histiocytic lymphoma (DHL), immunoblastic large cell lymphoma, precursor B-lymphoblastic lymphoma, solitary plasmocytoma, IgG myeloma, light chain myeloma, nonsecretory myeloma, amyloidosis, and any combinations of said hematological malignancies.
   88. The method of embodiment 84, wherein the hematological malignancy is an advanced, metastatic, refractory and/or recurrent hematological malignancy, and any combinations of said hematological malignancies.
   89. The method of any one of embodiments 59-88, which further comprises administering to the subject a therapeutically effective amount of an additional therapeutic agent for treating a cancer.
   90. The method of embodiment 89, wherein the additional therapeutic agent is a compound that reduces inhibition, or increases stimulation, of the immune system.
   91. The method of embodiment 90, wherein the additional therapeutic agent is:
(a) an antagonistic agent that binds specifically to Programmed Death-1 (PD-1), Programmed Death Ligand-1 (PD-L1), Cytotoxic T-Lymphocyte Antigen-4 (CTLA-4), Lymphocyte Activation Gene-3 (LAG-3), B and T lymphocyte Attenuator (BTLA), T cell Immunoglobulin and Mucin domain-3 (TIM-3), Killer Immunoglobulin-like Receptor (KIR), Killer cell Lectin-like Receptor G1 (KLRG-1), Adenosine A2a Receptor (A2aR), T Cell Immunoreceptor with Ig and ITIM Domains (TIGIT), V-domain Ig Suppressor of T cell activation (VISTA), proto-oncogene tyrosine-protein kinase MER (MerTK), Natural Killer Cell Receptor 2B4 (CD244), or CD160; or
(b) an agonistic agent that binds specifically to Inducible T cell Co-Stimulator (ICOS), CD137 (4-1BB), CD134 (OX40), CD27, Glucocorticoid-Induced TNFR-Related protein (GITR), or HerpesVirus Entry Mediator (HVEM).
   92. The method of embodiment 91, wherein the antagonistic agent that binds specifically to PD-1 is chosen from nivolumab, pembrolizumab, cemiplimab, spartalizumab, camrelizumab, sintilimab, tislelizumab, toripalimab, dostarlimab, retifanlimab, zimberelimab, pimivalimab, serplulimab, vopratelimab, and acrixolimab.
   93. The method of embodiment 91, wherein the antagonistic agent that binds specifically to PD-L1 is chosen from atezolizumab, durvalumab, avelumab, envafolimab, cosibelimab (CK-301), BMS-936559, BMS-986189, CS-1001, SHR-1316, CBT-502, BGB-A333, KN035, AUNP12, and CA-170.
   94. The method of embodiment 91, wherein the antagonistic agent that binds specifically to CTLA-4 is ipilimumab or tremelimumab.
   95. The method of embodiment 91, wherein the antagonistic agent that binds specifically to LAG3 is relatlimab, favezelimab, tiragolumab, fianlimab, tebotelimab or eftilagimod alpha.
   96. A kit for use in measuring a depletion in the number of Tregs in a subject, the kit comprising:
(a) a monoclonal antibody or an antigen-binding portion thereof that binds specifically to hCCR8 expressed on the surface of a cell, wherein the antibody or antigen-binding portion thereof binds to an epitope that is different from an epitope in the N-terminal domain of hCCR8; and
(b) instructions for using the monoclonal antibody or portion thereof in the method of any one of embodiments 49-53.
   97. A kit for use in predicting the effectiveness of a therapeutic anti-CCR8 antibody that binds to the N-terminal domain of hCCR8 in treating a cancer in a subject, the kit comprising:
(a) a monoclonal antibody or an antigen-binding portion thereof that binds specifically to hCCR8 expressed on the surface of a cell, wherein the antibody or antigen-binding portion thereof binds to an epitope that is different from an epitope in the N-terminal domain of hCCR8; and
(b) instructions for using the monoclonal antibody or portion thereof in the method of embodiment 55.
   98. A kit for use in selecting a subject afflicted with cancer as a suitable candidate for immunotherapy with a therapeutic anti-CCR8 antibody that binds to the N-terminal domain of hCCR8, the kit comprising:
(a) a monoclonal antibody or an antigen-binding portion thereof that binds specifically to hCCR8 expressed on the surface of a cell, wherein the antibody or antigen-binding portion thereof binds to an epitope that is different from an epitope in the N-terminal domain of hCCR8; and
(b) instructions for using the monoclonal antibody or portion thereof in the method of embodiment 56.
   99. A kit for use in treating a cancer in a subject, the kit comprising:
(a) a monoclonal antibody or an antigen-binding portion thereof that binds specifically to hCCR8 expressed on the surface of a cell, wherein the antibody or antigen-binding portion thereof binds to an epitope that is different from an epitope in the N-terminal domain of hCCR8;
(b) a therapeutic anti-CCR8 antibody or an antigen-binding portion thereof, that binds to the N-terminal domain of hCCR8; and
(c) instructions for using the monoclonal antibody or portion thereof and the therapeutic anti-CCR8 antibody or antigen-binding portion thereof in the method of embodiment 57 or 63.
   100. A kit for use in treating a cancer in a subject, the kit comprising:
(a) a monoclonal antibody or an antigen-binding portion thereof that binds specifically to hCCR8 expressed on the surface of a cell, wherein the antibody or antigen-binding portion thereof binds to an epitope that is different from an epitope in the N-terminal domain of hCCR8;
(b) a therapeutic anti-CCR8 antibody or an antigen-binding portion thereof, that binds to the N-terminal domain of hCCR8; and
(c) instructions for using the monoclonal antibody or portion thereof and the therapeutic anti-CCR8 antibody or antigen-binding portion thereof in the method of embodiment 61 or 65.

### Disclosed Amino Acid Sequences

The SEQ ID NOs. and corresponding amino acid sequences referred to in this application are summarized in Table 3.

TABLE 3
SEQ ID NOs. and Amino acid Sequence Summary

| SEQ ID | | |
|---|---|---|
| NO. | Description of Sequence | Amino Acid Sequence |
| 1 | Amino acid sequence of | MDYTLDLSVT TVTDYYYPDI FSSPCDAELI |
| | human CCR8 | QTNGKLLLAV FYCLLFVFSL LGNSLVILVL |
| | | VVCKKLRSIT DVYLLNLALS DLLFVFSFPF |
| | | QTYYLLDQWV FGTVMCKVVS GFYYIGFYSS |
| | | MFFITLMSVD RYLAVVHAVY ALKVRTIRMG |
| | | TTLCLAVWLT AIMATIPLLV FYQVASEDGV |
| | | LQCYSFYNQQ TLKWKIFTNF KMNILGLLIP |
| | | FTIFMFCYIK ILHQLKRCQN HNKTKAIRLV |
| | | LIVVIASLLF WVPFNVVLFL TSLHSMHILD |
| | | GCSISQQLTY ATHVTEIISF THCCVNPVIY |
| | | AFVGEKFKKH LSEIFQKSCS QIFNYLGRQM |
| | | PRESCEKSSS CQQHSSRSSS VDYIL |
| 2 | Amino acid residues 15-21 of | YYYPDIF |
| | human CCR8 sequence | |
| 3 | Amino Acid Sequence for V_{H} | Intentionally Left Blank |
| | in mAb 25T40 | |
| 4 | Amino Acid Sequence for V_{H} | VVQLQQSGPV LVKPGASVKM SCAASGYTFT |
| | in mAb 21C17 | DNYINWVKQR HGKSLEWIGV INPYNGLTAY |
| | | DQNFKGKATL TVDKSSSTAY MALNSLTSEA |
| | | SAVYYCARRY GGTPVRYFDV WGTGTTVTVS |
| | | S |
| 5 | Amino Acid Sequence for V_{H} | Intentionally Left Blank |
| | in mAb 28P3 | |
| 6 | Amino Acid Sequence for V_{H} | VVQLQQSGPV LVKPGASVKM SCAASGYTFT |
| | in mAb 22B13 | DNYINWVKQR HGKSLEWIGV INPYNGLTAY |
| | | DQNFKGKATL TVDKSSSTAY MALNSLTSEA |
| | | SAVYYCARRY GGTPVRYFDV WGTGTTVTVS |
| | | S |
| 7 | Amino Acid Sequence for V_{H} | Intentionally Left Blank |
| | in mAb 33H18 | |
| 8 | Amino Acid Sequence for V_{H} | QVQLVESGGG VVQPGRSLRL SCAVSGLTFS |
| | in mAb 23A14 | SYAMHWVRQA PGKGLEWVAV ISYDGNKKYN |
| | | ADSVKGRFTI SRDNSKNTLY LQMNSLRAED |
| | | TAVYYCARAE GKGDYWGQGT LVTVSS |
| 9 | Amino Acid Sequence for V_{H} | QVQLVQSGAE VKKPGASVKV SCKASGYTFT |
| | in mAb 4A19 | DSEMHWVRQA TGQGLEWMGA IQPETGGTAY |
| | | NQKFKARVTM TRDTSISTAY MELSSLRSED |
| | | TAVYYCARRR RNFDYWGQGT LVTVSS |
| 10 | Amino Acid Sequence for V_{L} | Intentionally Left Blank |
| | in mAb 25T40 | |
| 11 | Amino Acid Sequence for V_{L} | DVVLTQTPLT LSVTIGQPAS ISCKSSQSLL |
| | in mAb 21C17 | DSSGKTYLNW LLQRPGQSPK RLIYLVSKLD |
| | | SGVPDRFSGS GSGTDFTLKF SRVEAEDLGV |
| | | YYCWQATHFP WSFGGGSKLE VK |
| 12 | Amino Acid Sequence for V_{L} | Intentionally Left Blank |
| | in mAb 28P3 | |
| 13 | Amino Acid Sequence for V_{L} | DIQMTQTASS LSASLGARVT ISCRASQDIS |
| | in mAb 22B13 | NYLNWYQQKP DGTFELLIYY TSRLHSGVPS |
| | | RFSGSGSGTD YSLTITNLEQ EDIATYFCQQ |
| | | GETLPWTFGG GTKLEIK |
| 14 | Amino Acid Sequence for V_{L} | Intentionally Left Blank |
| | in mAb 33H18 | |
| 15 | Amino Acid Sequence for V_{L} | EIVLTQSPAT LSLSPGERAT LSCRASQSVS |
| | in mAb 23A14 | SYLVWYQQKP GQAPRLLIYD ASNRATGIPA |
| | | RFSGSGSGTD FTLTISSLEP EDFAVYYCQQ |
| | | RSNWPRTFGQ GTKVEIK |
| 16 | Amino Acid Sequence for V_{L} | DIVMTQTPLS LSVTPGQPAS ISCRSSQSLF |
| | in mAb 4A19 | HSSGNTYLHW YLQKPGQPPQ LLIYKVSNRF |
| | | SGVPDRFSGS GSGTDFTLKI SRVEAEDVGV |
| | | YYCSQSTHVP FTFGQGTKLE IK |
| 17 | Amino Acid Sequence for V_{H} | Intentionally Left Blank |
| | CDR1 in mAb 25T40 | |
| 18 | Amino Acid Sequence for V_{H} | Intentionally Left Blank |
| | CDR2 in mAb 25T40 | |
| 19 | Amino Acid Sequence for V_{H} | Intentionally Left Blank |
| | CDR3 in mAb 25T40 | |
| 20 | Amino Acid Sequence for V_{L} | Intentionally Left Blank |
| | CDR1 in mAb 25T40 | |
| 21 | Amino Acid Sequence for V_{L} | Intentionally Left Blank |
| | CDR2 in mAb 25T40 | |
| 22 | Amino Acid Sequence for V_{L} | Intentionally Left Blank |
| | CDR3 in mAb 25T40 | |
| 23 | Amino Acid Sequence for V_{H} | GYTFTDNYIN |
| | CDR1 in mAb 21C17 | |
| 24 | Amino Acid Sequence for V_{H} | VINPYNGLTA YDQNFKGKA |
| | CDR2 in mAb 21C17 | |
| 25 | Amino Acid Sequence for V_{H} | RYGGTPVRYF DV |
| | CDR3 in mAb 21C17 | |
| 26 | Amino Acid Sequence for V_{L} | KSSQSLLDSS GKTYLN |
| | CDR1 in mAb 21C17 | |
| 27 | Amino Acid Sequence for V_{L} | LVSKLDS |
| | CDR2 in mAb 21C17 | |
| 28 | Amino Acid Sequence for V_{L} | WQATHFPWS |
| | CDR3 in mAb 21C17 | |
| 29 | Amino Acid Sequence for V_{H} | Intentionally Left Blank |
| | CDR1 in mAb 28P3 | |
| 30 | Amino Acid Sequence for V_{H} | Intentionally Left Blank |
| | CDR2 in mAb 28P3 | |
| 31 | Amino Acid Sequence for V_{H} | Intentionally Left Blank |
| | CDR3 in mAb 28P3 | |
| 32 | Amino Acid Sequence for V_{L} | Intentionally Left Blank |
| | CDR1 in mAb 28P3 | |
| 33 | Amino Acid Sequence for V_{L} | Intentionally Left Blank |
| | CDR2 in mAb 28P3 | |
| 34 | Amino Acid Sequence for V_{L} | Intentionally Left Blank |
| | CDR3 in mAb 28P3 | |
| 35 | Amino Acid Sequence for V_{H} | GYTFTDNYIN |
| | CDR1 in mAb 22B13 | |
| 36 | Amino Acid Sequence for V_{H} | VINPYNGLTA YDQNFKGKA |
| | CDR2 in mAb 22B13 | |
| 37 | Amino Acid Sequence for V_{H} | RYGGTPVRYF DV |
| | CDR3 in mAb 22B13 | |
| 38 | Amino Acid Sequence for V_{L} | RASQDISNYL N |
| | CDR1 in mAb 22B13 | |
| 39 | Amino Acid Sequence for V_{L} | YTSRLHS |
| | CDR2 in mAb 22B13 | |
| 40 | Amino Acid Sequence for V_{L} | QQGETLPWT |
| | CDR3 in mAb 22B13 | |
| 41 | Amino Acid Sequence for V_{H} | Intentionally Left Blank |
| | CDR1 in mAb 33H18 | |
| 42 | Amino Acid Sequence for V_{H} | Intentionally Left Blank |
| | CDR2 in mAb 33H18 | |
| 43 | Amino Acid Sequence for V_{H} | Intentionally Left Blank |
| | CDR3 in mAb 33H18 | |
| 44 | Amino Acid Sequence for V_{L} | Intentionally Left Blank |
| | CDR1 in mAb 33H18 | |
| 45 | Amino Acid Sequence for V_{L} | Intentionally Left Blank |
| | CDR2 in mAb 33H18 | |
| 46 | Amino Acid Sequence for V_{L} | Intentionally Left Blank |
| | CDR3 in mAb 33H18 | |
| 47 | Amino Acid Sequence for V_{H} | GLTFSSYAMH |
| | CDR1 in mAb 23A14 | |
| 48 | Amino Acid Sequence for V_{H} | VISYDGNKKY NADSVKGRF |
| | CDR2 in mAb 23A14 | |
| 49 | Amino Acid Sequence for V_{H} | AEGKGDY |
| | CDR3 in mAb 23A14 | |
| 50 | Amino Acid Sequence for V_{L} | RASQSVSSYL V |
| | CDR1 in mAb 23A14 | |
| 51 | Amino Acid Sequence for V_{L} | DASNRAT |
| | CDR2 in mAb 23A14 | |
| 52 | Amino Acid Sequence for V_{L} | QQRSNWPRT |
| | CDR3 in mAb 23A14 | |
| 53 | Amino Acid Sequence for V_{H} | GYTFTDSEMH |
| | CDR1 in mAb 4A19 | |
| 54 | Amino Acid Sequence for V_{H} | AIQPETGGTA YNQKFKA |
| | CDR2 in mAb 4A19 | |
| 55 | Amino Acid Sequence for V_{H} | RRRNFDY |
| | CDR3 in mAb 4A19 | |
| 56 | Amino Acid Sequence for V_{L} | RSSQSLFHSS GNTYLH |
| | CDR1 in mAb 4A19 | |
| 57 | Amino Acid Sequence for V_{L} | KVSNRFS |
| | CDR2 in mAb 4A19 | |
| 58 | Amino Acid Sequence for V_{L} | SQSTHVPFT |
| | CDR3 in mAb 4A19 | |
| 59 | Amino Acid Sequence for | Intentionally Left Blank |
| | heavy chain mAb of 25T40 | |
| 60 | Amino Acid Sequence for | VVQLQQSGPV LVKPGASVKM SCAASGYTFT |
| | heavy chain mAb of 21C17 | DNYINWVKQR HGKSLEWIGV INPYNGLTAY |
| | | DQNFKGKATL TVDKSSSTAY MALNSLTSEA |
| | | SAVYYCARRY GGTPVRYFDV WGTGTTVTVS |
| | | SAKTTPPSVY PLAPGSAAQT NSMVTLGCLV |
| | | KGYFPEPVTV TWNSGSLSSG VHTFPAVLQS |
| | | DLYTLSSSVT VPSSTWPSET VTCNVAHPAS |
| | | STKVDKKIVP RDCGCKPCIC TVPEVSSVFI |
| | | FPPKPKDVLT ITLTPKVTCV VVAISKDDPE |
| | | VQFSWFVDDV EVHTAQTQPR EEQFNSTFRS |
| | | VSELPIMHQD WLNGKEFKCR VNSAAFPAPI |
| | | EKTISKTKGR PKAPQVYTIP PPKEQMAKDK |
| | | VSLTCMITDF FPEDITVEWQ WNGQPAENYK |
| | | NTQPIMDTDG SYFVYSKLNV QKSNWEAGNT |
| | | FTCSVLHEGL HNHHTEKSLS HSPG |
| 61 | Amino Acid Sequence for | Intentionally Left Blank |
| | heavy chain mAb of 28P3 | |
| 62 | Amino Acid Sequence for | VVQLQQSGPV LVKPGASVKM SCAASGYTFT |
| | heavy chain mAb of 22B13 | DNYINWVKQR HGKSLEWIGV INPYNGLTAY |
| | | DQNFKGKATL TVDKSSSTAY MALNSLTSEA |
| | | SAVYYCARRY GGTPVRYFDV WGTGTTVTVS |
| | | SAKTTPPSVY PLAPGSAAQT NSMVTLGCLV |
| | | KGYFPEPVTV TWNSGSLSSG VHTFPAVLQS |
| | | DLYTLSSSVT VPSSTWPSET VTCNVAHPAS |
| | | STKVDKKIVP RDCGCKPCIC TVPEVSSVFI |
| | | FPPKPKDVLT ITLTPKVTCV VVAISKDDPE |
| | | VQFSWFVDDV EVHTAQTQPR EEQFNSTFRS |
| | | VSELPIMHQD WLNGKEFKCR VNSAAFPAPI |
| | | EKTISKTKGR PKAPQVYTIP PPKEQMAKDK |
| | | VSLTCMITDF FPEDITVEWQ WNGQPAENYK |
| | | NTQPIMDTDG SYFVYSKLNV QKSNWEAGNT |
| | | FTCSVLHEGL HNHHTEKSLS HSPG |
| 63 | Amino Acid Sequence for | Intentionally Left Blank |
| | heavy chain mAb of 33H18 | |
| 64 | Amino Acid Sequence for | QVQLVESGGG VVQPGRSLRL SCAVSGLTFS |
| | heavy chain mAb of 23A14 | SYAMHWVRQA PGKGLEWVAV ISYDGNKKYN |
| | | ADSVKGRFTI SRDNSKNTLY LQMNSLRAED |
| | | TAVYYCARAE GKGDYWGQGT LVTVSSASTK |
| | | GPSVFPLAPS SKSTSGGTAA LGCLVKDYFP |
| | | EPVTVSWNSG ALTSGVHTFP AVLQSSGLYS |
| | | LSSVVTVPSS SLGTQTYICN VNHKPSNTKV |
| | | DKRVEPKSCD KTHTCPPCPA PEAEGAPSVF |
| | | LFPPKPKDTL MISRTPEVTC VVVDVSHEDP |
| | | EVKFNWYVDG VEVHNAKTKP REEQYNSTYR |
| | | VVSVLTVLHQ DWLNGKEYKC KVSNKALPAP |
| | | IEKTISKAKG QPREPQVYTL PPSREEMTKN |
| | | QVSLTCLVKG FYPSDIAVEW ESNGQPENNY |
| | | KTTPPVLDSD GSFFLYSKLT VDKSRWQQGN |
| | | VFSCSVMHEA LHNHYTQKSL SLSPG |
| 65 | Amino Acid Sequence for | QVQLVQSGAE VKKPGASVKV SCKASGYTFT |
| | heavy chain mAb of 4A19 | DSEMHWVRQA TGQGLEWMGA IQPETGGTAY |
| | | NQKFKARVTM TRDTSISTAY MELSSLRSED |
| | | TAVYYCARRR RNFDYWGQGT LVTVSSASTK |
| | | GPSVFPLAPS SKSTSGGTAA LGCLVKDYFP |
| | | EPVTVSWNSG ALTSGVHTFP AVLQSSGLYS |
| | | LSSVVTVPSS SLGTQTYICN VNHKPSNTKV |
| | | DKRVEPKSCD KTHTCPPCPA PELLGGPSVF |
| | | LFPPKPKDTL MISRTPEVTC VVVDVSHEDP |
| | | EVKFNWYVDG VEVHNAKTKP REEQYNSTYR |
| | | VVSVLTVLHQ DWLNGKEYKC KVSNKALPAP |
| | | IEKTISKAKG QPREPQVYTL PPSREEMTKN |
| | | QVSLTCLVKG FYPSDIAVEW ESNGQPENNY |
| | | KTTPPVLDSD GSFFLYSKLT VDKSRWQQGN |
| | | VFSCSVMHEA LHNHYTQKSL SLSPG |
| 66 | Amino Acid Sequence for | Intentionally Left Blank |
| | light chain mAb of 25T40 | |
| 67 | Amino Acid Sequence for | DVVLTQTPLT LSVTIGQPAS ISCKSSQSLL |
| | light chain mAb of 21C17 | DSSGKTYLNW LLQRPGQSPK RLIYLVSKLD |
| | | SGVPDRFSGS GSGTDFTLKF SRVEAEDLGV |
| | | YYCWQATHFP WSFGGGSKLE VKRADAAPTV |
| | | SIFPPSSEQL TSGGASVVCF LNNFYPKDIN |
| | | VKWKIDGSER QNGVLNSWTD QDSKDSTYSM |
| | | SSTLTLTKDE YERHNSYTCE ATHKTSTSPI |
| | | VKSFNRNEC |
| 68 | Amino Acid Sequence for | Intentionally Left Blank |
| | light chain mAb of 28P3 | |
| 69 | Amino Acid Sequence for | DIQMTQTASS LSASLGARVT ISCRASQDIS |
| | | NYLNWYQQKP DGTFELLIYY TSRLHSGVPS |
| | light chain mAb of 22B13 | RFSGSGSGTD YSLTITNLEQ EDIATYFCQQ |
| | | GETLPWTFGG GTKLEIKRAD AAPTVSIFPP |
| | | SSEQLTSGGA SVVCFLNNFY PKDINVKWKI |
| | | DGSERQNGVL NSWTDQDSKD STYSMSSTLT |
| | | LTKDEYERHN SYTCEATHKT STSPIVKSFN |
| | | RNEC |
| 70 | Amino Acid Sequence for | Intentionally Left Blank |
| | light chain mAb of 33H18 | |
| 71 | Amino Acid Sequence for | EIVLTQSPAT LSLSPGERAT LSCRASQSVS |
| | light chain mAb of 23A14 | SYLVWYQQKP GQAPRLLIYD ASNRATGIPA |
| | | RFSGSGSGTD FTLTISSLEP EDFAVYYCQQ |
| | | RSNWPRTFGQ GTKVEIKRTV AAPSVFIFPP |
| | | SDEQLKSGTA SVVCLLNNFY PREAKVQWKV |
| | | DNALQSGNSQ ESVTEQDSKD STYSLSSTLT |
| | | LSKADYEKHK VYACEVTHQG LSSPVTKSFN |
| | | RGEC |
| 72 | Amino Acid Sequence for | DIVMTQTPLS LSVTPGQPAS ISCRSSQSLF |
| | light chain mAb of 4A19 | HSSGNTYLHW YLQKPGQPPQ LLIYKVSNRF |
| | | SGVPDRFSGS GSGTDFTLKI SRVEAEDVGV |
| | | YYCSQSTHVP FTFGQGTKLE IKRTVAAPSV |
| | | FIFPPSDEQL KSGTASVVCL LNNFYPREAK |
| | | VQWKVDNALQ SGNSQESVTE QDSKDSTYSL |
| | | SSTLTLSKAD YEKHKVYACE VTHQGLSSPV |
| | | TKSFNRGEC |
| 73 | Amino acid residues 12-22 of | VTDYYYPDIF S |
| | human CCR8 sequence | |
| 74 | Amino acid residues 1-35 of | MDYTLDLSVT TVTDYYYPDI FSSPCDAELI |
| | human CCR8 sequence | QTNGK |

### REFERENCES

Abhinandan K R, Martin A C (2008) Analysis and improvements to Kabat and structurally correct numbering of antibody variable domains. Mol Immunol 45:3832-9.
Al-Lazikani, Lesk A M, Chothia C (1997) Standard conformations for the canonical structures of immunoglobulins. J Mol Biol 273(4):927-48.
Aluicio-Sarduy E, Ellison P A, Barnhart T E, Cai W, Nickles R J, Engle J W (2018) PET radiometals for antibody labeling. J Label Compd Radiopharm 61:636-51.
Baumeister S H, Freeman G J, Dranoff G, Sharpe A H (2016) Coinhibitory pathways in immunotherapy for cancer. Annu Rev Immunol 34:539-73.
Brahmer J R, Hammers H, Lipson E J (2015) Nivolumab: targeting PD-1 to bolster antitumor immunity. Future Oncol 11(9):1307-26.
Callahan M, Postow M A, Wolchok J D (2016) Targeting T cell co-receptors for cancer therapy. Immunity 44(5):1069-78.
Chothia C, Lesk A M (1987) Canonical structures for the hypervariable regions of immunoglobulins. J Mol Biol 196:901-17.
Chothia C, Lesk A M, Tramontano A, Levitt M, Smith-Gill J S et al. (1989) Conformations of immunoglobulin hypervariable regions. Nature 342:877-83.
De Simone M, Arrigoni A, Rossetti G et al. (2016) Transcriptional landscape of human tissue lymphocytes unveils uniqueness of tumor-infiltrating T regulatory cells. Immunity 5:1135-47.
Drugs.com—Opdivo Approval History: https://www.drugs.com/history/opdivo.html, last accessed May 22, 2023.
Farkona et al. (2016) Cancer immunotherapy: the beginning of the end of cancer? BMC Medicine 14:73.
Fares C M, Van Allen E M, Drake C G, Allison J P, Hu-Lieskovan S (2019) Mechanisms of resistance to immune checkpoint blockade: why does checkpoint inhibitor immunotherapy not work for all patients? ASCO Education Book 39:147-64.
Finotello F, Trajanoski Z (2017) New strategies for cancer immunotherapy: targeting regulatory T cells. Genome Medicine 9:10.
Gadalla R, Noamani B, MacLeod B L, Dickson R J, Guo M et al. (2019) Validation of CyTOF against flow cytometry for immunological studies and monitoring of human cancer clinical trials. Front Oncol 9:415.
Guo L, Zhang H, Chen B (2017) Nivolumab as Programmed Death-1 (PD-1) inhibitor for targeted immunotherapy in tumor. J Cancer 8(3):410-416.
Han S, Toker A, Liu Z Q, Ohashi P S (2019) Turning the tide against regulatory T cells. Front Oncol 9: 279.
Haslam A, Prasad V (2019) Estimation of the Percentage of U S Patients With Cancer Who Are Eligible for and Respond to Checkpoint Inhibitor Immunotherapy Drugs. JAMA Netw Open 2(5): e192535.
Kabat E A, Wu T T, Bilofsky H, Reid-Miller M, Perry H (1983) Sequence of proteins of immunological interest. Bethesda: National Institute of Health; 1983. 323.
Kamta J, Chaar M, Ande A, Altomare D A, Ait-Oudhia S (2017) Advancing cancer therapy with present and emerging immuno-oncology approaches. Front Oncol 18(7):64.
Kaufman R J, Sharp P A (1982) Amplification and expression of sequences cotransfected with a modular dihydrofolate reductase complementary DNA gene. Mol Biol 159:601-21.
Lefranc M P, Pommie C, Ruiz M, Giudicelli V, Foulquier E et al. (2003) IMGT unique numbering for immunoglobulin and T cell receptor variable domains and Ig superfamily V-like domains. Dev Comp Immunol 27:55-77.
Lesokhin A M, Callahan M K, Postow M A, Wolchok J D (2015) On being less tolerant: enhanced cancer immunosurveillance enabled by targeting checkpoints and agonists of T cell activation. Sci Transl Med 7(280):280sr1.
Martin A, Cheetham J C, Rees A R (1989) Modeling antibody hypervariable loops: a combined algorithm. Proc Natl Acad Sci USA 86(23):9268-72.
MacCallum R M., Martin A C R, Thornton J T (1996) Antibody-antigen interactions: contact analysis and binding site topography. J Mol Biol 262:732-45.
Pardoll D M (2012) The blockage of immune checkpoints in cancer immunotherapy. Nat Rev Cancer 12: 252-64.
PCT Publication No. WO 2007/044756, published Apr. 19, 2007 by ICOS Corp.
PCT Publication No. WO 2013/173223, published Nov. 21, 2013 by Bristol-Myers Squibb Co.
PCT Publication No. WO 2020/138489, published Jul. 2, 2020 by Shionogi & Co. Ltd. and Osaka University.
PCT Publication No. WO 2021/142002, published Jul. 15, 2021 by Vaccinex, Inc.
PCT Publication No. WO 2021/152186, published Aug. 5, 2021 by Bayer Aktikngeskllschaft and Bayer Healthcare.
PCT Publication No. WO 2021/163064, published Aug. 19, 2021 by Jounce Therapeutics, Inc.
PCT Publication No. WO 2021/194942, published Sep. 30, 2021 by Bristol-Myers Squib Company.
PCT Publication No. WO 2021/260209, published Dec. 30, 2021 by Bayer Aktikngeskllschaft and Bayer Healthcare.
PCT Publication No. WO 2022/003156, published Jan. 6, 2022 by Oncurious N V
PCT Publication No. WO 2022/136647, published Jun. 30, 2022 by Oncurious N V
PCT Publication No. WO 2022/136649, published Jun. 30, 2022 by Oncurious N V et al.
Pianko M J, Liu Y, Bagchi S, Lesokhin A M (2017) Immune checkpoint blockade for hematologic malignancies: a review. Stem Cell Investig 4:32.
Plitas G, Konopacki C, Wu K et al. (2016) Regulatory T cells exhibit distinct features in human breast cancer. Immunity 5:1122-34.
Sandland J, Boyle R W (2019) Photosensitizer antibody—drug conjugates: past, present, and future. Bioconjugate Chem 30(4):975-93.
Spitzer M H, Nolan G P (2016) Mass cytometry: single cells, many features. Cell 165(4):780-91.
U.S. Pat. No. 7,767,429, issued Aug. 3, 2010 to Bookbinder et al.
Wang L, Simons D L, Lu X et al. (2019) Connecting blood and intratumoral Treg cell activity in predicting future relapse in breast cancer. Nature Immunol 20:1220-30.
Wu T T, Kabat E A (1970) An analysis of the sequences of the variable regions of Bence Jones proteins and myeloma light chains and their implications for antibody complementarity. J Exp Med 132:211-250.
Yao S, Zhu Y, Chen L (2013) Advances in targeting cell surface signalling molecules for immune modulation. Nature Rev Drug Discov 12:130-46.

1. A monoclonal antibody, or an antigen-binding portion thereof, that binds specifically to human C-C Motif Chemokine Receptor 8 (hCCR8) expressed on the surface of a cell, wherein the sequence of hCCR8 is set forth as SEQ ID NO: 1, and wherein the antibody or antigen-binding portion thereof binds to an epitope that is not in the N-terminal domain of hCCR8 and does not cross-compete with an antibody that binds to an N-terminal epitope for binding to hCCR8.

2. The monoclonal antibody or antigen-binding portion thereof of claim 1, wherein the N-terminal epitope comprises at least the amino acids having the sequence Y₁₅Y₁₆Y₁₇P₁₈D₁₉I₂₀F₂₁ (SEQ ID NO: 2), or all eleven of the amino acids having the sequence V₁₂T₁₃D₁₄Y₁₅Y₁₆Y₁₇P₁₈D₁₉I₂₀F₂₁S₂₂ (SEQ ID NO: 73), wherein amino acids Y₁₅ and/or Y₁₇ are sulfated.

3. The monoclonal antibody or antigen-binding portion thereof of claim 1, which:
comprises the following CDR domains as defined by the Kabat method,
(a) a heavy chain variable region CDR1 comprising consecutively linked amino acids having the sequence set forth as SEQ ID NO: 23; a heavy chain variable region CDR2 comprising consecutively linked amino acids having the sequence set forth as SEQ ID NO: 24; a heavy chain variable region CDR3 comprising consecutively linked amino acids having the sequence set forth as SEQ ID NO: 25; a light chain variable region CDR1 comprising consecutively linked amino acids having the sequence set forth as SEQ ID NO: 26; a light chain variable region CDR2 comprising consecutively linked amino acids having the sequence set forth as SEQ ID NO: 27; and a light chain variable region CDR3 comprising consecutively linked amino acids having the sequence set forth as SEQ ID NO: 28;
(b) a heavy chain variable region CDR1 comprising consecutively linked amino acids having the sequence set forth as SEQ ID NO: 35; a heavy chain variable region CDR2 comprising consecutively linked amino acids having the sequence set forth as SEQ ID NO: 36; a heavy chain variable region CDR3 comprising consecutively linked amino acids having the sequence set forth as SEQ ID NO: 37; a light chain variable region CDR1 comprising consecutively linked amino acids having the sequence set forth as SEQ ID NO: 38; a light chain variable region CDR2 comprising consecutively linked amino acids having the sequence set forth as SEQ ID NO: 39; and a light chain variable region CDR3 comprising consecutively linked amino acids having the sequence set forth as SEQ ID NO: 40; or
(c) a heavy chain variable region CDR1 comprising consecutively linked amino acids having the sequence set forth as SEQ ID NO: 47; a heavy chain variable region CDR2 comprising consecutively linked amino acids having the sequence set forth as SEQ ID NO: 48; a heavy chain variable region CDR3 comprising consecutively linked amino acids having the sequence set forth as SEQ ID NO: 49; a light chain variable region CDR1 comprising consecutively linked amino acids having the sequence set forth as SEQ ID NO: 50; a light chain variable region CDR2 comprising consecutively linked amino acids having the sequence set forth as SEQ ID NO: 51; and a light chain variable region CDR3 comprising consecutively linked amino acids having the sequence set forth as SEQ ID NO: 22;
comprises the following heavy and light chain variable regions,
(d) a V_{H} comprising consecutively linked amino acids having the sequence set forth as SEQ ID NO: 4 and a V_{L} comprising consecutively linked amino acids having the sequence set forth as SEQ ID NO: 11;
(e) a V_{H} comprising consecutively linked amino acids having the sequence set forth as SEQ ID NO: 6 and a V_{L} comprising consecutively linked amino acids having the sequence set forth as SEQ ID NO: 13; or
(f) a V_{H} comprising consecutively linked amino acids having the sequence set forth as SEQ ID NO: 8 and a V_{L} comprising consecutively linked amino acids having the sequence set forth as SEQ ID NO: 15; and/or
comprises the following heavy and light chains,
(g) a heavy chain comprising consecutively linked amino acids having the sequence set forth as SEQ ID NO: 60 and a light chain comprising consecutively linked amino acids having the sequence set forth as SEQ ID NO: 67;
(h) a heavy chain comprising consecutively linked amino acids having the sequence set forth as SEQ ID NO: 62 and a light chain comprising consecutively linked amino acids having the sequence set forth as SEQ ID NO: 69; or
(i) a heavy chain comprising consecutively linked amino acids having the sequence set forth as SEQ ID NO: 64 and a light chain comprising consecutively linked amino acids having the sequence set forth as SEQ ID NO: 71.

4. The monoclonal antibody or antigen-binding thereof of claim 1, labeled with a detectable label.

5. An immunoconjugate comprising the monoclonal antibody or antigen-binding portion thereof of claim 1, linked to a cytolytic agent.

6. (canceled)

7. An isolated nucleic acid encoding the monoclonal antibody or antigen-binding portion thereof of claim 1; or
an expression vector comprising said nucleic acid; or
a host cell comprising said expression vector; or
a method for preparing an anti-CCR8 antibody or antigen-binding portion thereof which comprises expressing the antibody or antigen-binding portion thereof in the host cell and isolating the antibody or antigen-binding portion thereof from the host cell.

8. A method for generating a first antibody that does not bind, or does not cross-compete with a second antibody for binding, to a defined epitope on an antigen, which comprises immunizing a vertebrate with an immunogen comprising a cell line, or a component of said cell line, that expresses the antigen and also expresses a second antibody or antigen-binding portion thereof that binds specifically to the epitope, wherein the binding of the second antibody or antigen-binding portion thereof to the epitope shields said epitope from the immune system of the vertebrate, reducing the generation of antibodies that bind to the epitope and thereby preferentially resulting in the generation of a first antibody that does not bind, or does not cross-compete with the second antibody for binding, to the epitope.

9. A method for measuring receptor occupancy (RO) of a cell membrane-bound CCR8 receptor to which a therapeutic Treg-depleting anti-CCR8 antibody binds comprising:
(a) adding to background whole blood samples, previously exposed to various concentrations of the therapeutic antibody, a saturating concentration of the therapeutic antibody;
(b) incubating aliquots of free, bound or fluorescence minus one (FMO) samples with buffer not containing the therapeutic antibody;
(c) staining the samples with a core panel of antibodies to identify basic T cell markers, T cell differentiation markers, and Treg markers;
(d) adding to the core panel
(i) for the direct/total RO assay, an anti-idiotypic antibody for detecting bound CCR8 receptor, or
(ii) for the indirect/total assay, two additional antibodies comprising an allophycocyanin (APC)-conjugated anti-hCCR8 antibody that competes with the therapeutic antibody, for detecting free CCR8 receptors, and an anti-hCCR8 antibody that does not compete with the therapeutic antibody, for detecting total CCR8 receptors;
(e) lysing the red blood cells to clear the samples and analyzing the cleared samples by flow cytometry; and
(f) determining the % RO for each concentration of the therapeutic antibody using the formula:
% RO=100×[1−((free postdose/free predose)/(total postdose/total predose))].

10. A method for measuring a depletion in the frequency of CCR8-expressing cells in the peripheral blood of a subject undergoing treatment with an anti-CCR8 mAb, the method comprising:
(a) determining a baseline level of expression of CCR8, and/or the frequency of CCR8-expressing cells in a first sample of whole blood or PBMCs from the subject;
(b) administering a treatment of an anti-CCR8 mAb to the subject; and
(c) determining the level of expression of CCR8, and/or the frequency of CCR8-expressing cells in a second sample of whole blood or PBMCs from the subject taken after administration of the anti-CCR8 mAb;
wherein a decrease in the level of expression of CCR8, and/or the frequency of CCR8-expressing cells in the second sample indicates that the number of CCR8-expressing cells in the blood has been depleted.

11. A method for measuring a depletion in the number of tumor-infiltrating CCR8-expressing Tregs in a subject undergoing treatment, the method comprising:
(a) determining a baseline level of expression of CCR8, and/or the frequency of CCR8-expressing Tregs, in a first sample of test tissue in or from the subject, the test tissue comprising tumor cells and tumor-infiltrating Tregs;
(b) administering a treatment of an anti-CCR8 antibody to the subject; and
(c) determining the level of expression of CCR8, and/or the frequency of CCR8-expressing Tregs, in a second sample of test tissue in or from a subject, the second test tissue taken during or after the treatment;
wherein a decrease in the level of expression of CCR8, and/or a decrease in the frequency of CCR8-expressing Tregs, in the second sample indicates that the number of Tregs in the test tissue has been depleted.

12. (canceled)

13. A method for predicting the effectiveness of a therapeutic Treg-depleting antibody or an antigen-binding portion thereof in treating cancer in a subject, which method comprises:
(a) determining the level of surface expression of CCR8, and/or the frequency of CCR8-expressing Tregs, in a test tissue in or taken from the subject, the test tissue comprising tumor cells and tumor-infiltrating Tregs;
(b) administering the therapeutic Treg-depleting antibody or antigen-binding portion thereof the subject;
(c) determining whether there is a decrease in the frequency of CCR8-expressing Tregs after administration of the therapeutic Treg-depleting antibody or antigen-binding portion thereof, and
(d) predicting that the therapeutic Treg-depleting antibody or antigen-binding portion thereof will be effective in treating cancer in the subject if the decrease in the frequency of CCR8-expressing Tregs exceeds a predetermined threshold value, or
(e) predicting that the therapeutic Treg-depleting antibody or antigen-binding portion thereof will not be effective in treating cancer in the subject if the decrease in the frequency of CCR8-expressing Tregs is less than a predetermined threshold value.

14. (canceled)

15. A method for treating a cancer in a subject, which method comprises:
(a) selecting a subject that is a suitable candidate for cancer immunotherapy with a therapeutic Treg-depleting antibody or an antigen-binding portion thereof, the selecting comprising:
(i) determining the level of surface expression of CCR8, and/or the frequency of CCR8-expressing Tregs, in a test tissue in or taken from the subject, the test tissue comprising tumor cells and tumor-infiltrating Tregs;
(ii) administering the therapeutic Treg-depleting antibody or antigen-binding portion thereof to the subject;
(iii) determining whether there is a decrease in the frequency of CCR8-expressing Tregs after administration of the therapeutic Treg-depleting antibody or antigen-binding portion thereof, and
(iv) selecting the subject as a suitable candidate for immunotherapy with the therapeutic based on an assessment that a decrease in the frequency of CCR8-expressing Tregs exceeds a predetermined threshold value; and
(b) administering to the selected subject a composition comprising a therapeutically effective amount of the therapeutic Treg-depleting antibody or antigen-binding portion thereof.

16. The method of claim 15, wherein:
(a) the cancer is a solid tumor,
wherein the solid tumor is a cancer chosen from squamous cell carcinoma, small cell lung cancer (SCLC), non-small cell lung cancer (NSCLC), squamous NSCLC, non-squamous NSCLC, head and neck cancer, breast cancer, cancer of the esophagus, gastric cancer, gastrointestinal cancer, cancer of the small intestine, liver cancer, hepatocellular carcinoma (HCC), pancreatic cancer (PAC), kidney cancer, renal cell carcinoma (RCC), bladder cancer, cancer of the urethra, cancer of the ureter, colorectal cancer (CRC), colon cancer, colon carcinoma, cancer of the anal region, endometrial cancer, prostate cancer, a fibrosarcoma, neuroblastoma, glioma, glioblastoma, germ cell tumor, pediatric sarcoma, sinonasal natural killer, melanoma, skin cancer, bone cancer, cervical cancer, uterine cancer, carcinoma of the endometrium, carcinoma of the fallopian tubes, ovarian cancer, carcinoma of the cervix, carcinoma of the vagina, carcinoma of the vulva, testicular cancer, cancer of the endocrine system, thyroid cancer, cancer of the parathyroid gland, cancer of the adrenal gland, sarcoma of soft tissue, cancer of the penis, carcinoma of the renal pelvis, neoplasm of the central nervous system (CNS), primary CNS lymphoma, tumor angiogenesis, spinal axis tumor, brain cancer, brain stem glioma, pituitary adenoma, Kaposi's sarcoma, epidermoid cancer, squamous cell cancer, solid tumors of childhood, environmentally-induced cancers, virus-related cancers, cancers of viral origin, advanced cancer, unresectable cancer, metastatic cancer, refractory cancer, recurrent cancer, and any combination thereof, or
(b) the cancer is a hematological malignancy,
wherein the hematological malignancy is selected from diffuse large B-cell lymphoma (DLBCL), CLL/small lymphocytic lymphoma (SLL), mantle cell lymphoma, mucosa-associated lymphoid tissue (MALT) lymphoma, nodal marginal zone B-cell lymphoma, splenic marginal zone B-cell lymphoma, Burkitt lymphoma, lymphoplasmacytoid lymphoma (LPL), hairy cell lymphoma, primary central nervous system (CNS) lymphoma, precursor T-lymphoblastic lymphoma/leukemia, T-lymphoblastic lymphoma/leukemia (T-Lbly/T-ALL), cutaneous T-cell lymphoma, adult T-cell lymphoma/leukemia, angioimmunoblastic T-cell lymphoma, extranodal natural killer/T-cell lymphoma nasal type, enteropathy-associated intestinal T-cell lymphoma (EATL), anaplastic large-cell lymphoma (ALCL), peripheral T-cell lymphoma unspecified, lymphoplasmacytoid lymphoma, monocytoid B cell lymphoma, angiocentric lymphoma, intestinal T-cell lymphoma, primary mediastinal B-cell lymphoma, post-transplantation lymphoproliferative disorder, true histiocytic lymphoma, primary effusion lymphoma, diffuse histiocytic lymphoma (DHL), immunoblastic large cell lymphoma, precursor B-lymphoblastic lymphoma, solitary plasmocytoma, IgG myeloma, light chain myeloma, nonsecretory myeloma, amyloidosis, and any combinations of said hematological malignancies.

17. (canceled)

18. A kit for use in treating a cancer in a subject, the kit comprising:
(a) a monoclonal antibody or an antigen-binding portion thereof that binds specifically to hCCR8 expressed on the surface of a cell, wherein the antibody or antigen-binding portion thereof binds to an epitope that is different from an epitope in the N-terminal domain of hCCR8;
(b) a therapeutic anti-CCR8 antibody or an antigen-binding portion thereof, that binds to the N-terminal domain of hCCR8; and
(c) instructions for using the monoclonal antibody or portion thereof and the therapeutic anti-CCR8 antibody or antigen-binding portion thereof in the method of claim 15.

19. The monoclonal antibody or antigen-binding portion thereof of claim 1, wherein the antibody that binds to the N-terminal epitope is an antibody comprising:
(a) a heavy chain variable region CDR1 comprising consecutively linked amino acids having the sequence set forth as SEQ ID NO: 53; a heavy chain variable region CDR2 comprising consecutively linked amino acids having the sequence set forth as SEQ ID NO: 54; a heavy chain variable region CDR3 comprising consecutively linked amino acids having the sequence set forth as SEQ ID NO: 55; a light chain variable region CDR1 comprising consecutively linked amino acids having the sequence set forth as SEQ ID NO: 56; a light chain variable region CDR2 comprising consecutively linked amino acids having the sequence set forth as SEQ ID NO: 57; and a light chain variable region CDR3 comprising consecutively linked amino acids having the sequence set forth as SEQ ID NO: 58;
(b) a V_{H} comprising consecutively linked amino acids having the sequence set forth as SEQ ID NO: 9 and a V_{L} comprising consecutively linked amino acids having the sequence set forth as SEQ ID NO: 16; and/or
(c) a heavy chain comprising consecutively linked amino acids having the sequence set forth as SEQ ID NO: 65 and a light chain comprising consecutively linked amino acids having the sequence set forth as SEQ ID NO: 72.

20. The labeled monoclonal antibody or antigen-binding thereof of claim 4, wherein the detectable label is a fluorophore, a chromophore, an enzyme, a radioactive isotope, a micropolymer, or a metal.

21. The labeled monoclonal antibody or antigen-binding thereof of claim 20, wherein:
(a) the detectable label is biotin,
(b) the fluorophore is a Brilliant Violet™ dye (e.g., BV-421), an AmCyan dye, an Alexa Fluor® dye, a Cy® dye, a CF® dye, fluorescein isothiocyanate (FITC), tetramethylrhodamine (TRITC) phycoerythrin (PE), allophycocyanin (APC), or peridinin-chlorophyll protein (PerCP),
(c) the chromophore is a porphyrin, pyropheophorbide-α, benzoporphyrin monoacid ring A (BPDMA), or chlorin e6,
(d) the enzyme is alkaline phosphatase, horseradish peroxidase, glucose oxidase or β-galactosidase,
(e) the radioactive isotope is ⁸⁹Zr, ⁶⁴Cu, ⁸⁶Y, ¹¹C, ¹⁸F, ⁶⁸Ga, ⁵²Mn, ⁵⁵Co, ¹⁵²Tb, ⁹⁰Nb, ⁶⁶Ga, ⁷²As, I¹²⁵, or ⁶⁹Ge, or
(f) the metal label is yttrium (Y), indium (In), the series of lanthanide elements (Ln, from La to Lu, except Pm), iodine (I), cadmium (Cd), tellurium (Te), silver (Ag), palladium (Pd), rhodium (Rh), iridium (Ir), platinum (Pt), ruthenium (Ru), osmium (Os) or bismuth (Bi).

22. The immunoconjugate of claim 5, wherein the cytolytic agent is a cytotoxin, a radioactive isotope, or a photosensitizer.

23. A chimeric antigen receptor (CAR) comprising the monoclonal antibody or antigen-binding portion thereof of claim 1.

24. A T cell receptor (TCR) comprising the monoclonal antibody or antigen-binding portion thereof of claim 1.

25. A bispecific molecule comprising the monoclonal antibody or antigen-binding portion thereof of claim 1, linked to a binding domain that has a different binding specificity than the monoclonal antibody or antigen binding portion thereof.

26. The method of claim 17, wherein the additional therapeutic agent is a compound that reduces inhibition, or increases stimulation, of the immune system.
